SEQUENCE LISTING (31 sequences)

SEQ ID NO: 1 - DNA, 11 bp, Artificial Sequence
  Description of Artificial Sequence Synthetic oligonucleotide
gccnnnnnggc

SEQ ID NO: 2 - DNA, 12 bp, Artificial Sequence
  Description of Artificial Sequence Synthetic oligonucleotide
cgannnnnntgc

SEQ ID NO: 3 - DNA, 21 bp, Artificial Sequence
  Description of Artificial Sequence Synthetic primer
gaatgcgaaactcagagatca

SEQ ID NO: 4 - DNA, 21 bp, Artificial Sequence
  Description of Artificial Sequence Synthetic primer
cctgtaatttctgtgcctcct

SEQ ID NO: 5 - DNA, 24 bp, Artificial Sequence
  Description of Artificial Sequence Synthetic probe
actgaagccnaaaaatggccattc

SEQ ID NO: 6 - DNA, 21 bp, Artificial Sequence
  Description of Artificial Sequence Synthetic primer
aaatgcttactgaagccgaaa

SEQ ID NO: 7 - DNA, 21 bp, Artificial Sequence
  Description of Artificial Sequence Synthetic primer
cgggtatttctctctgtgcat

SEQ ID NO: 8 - DNA, 24 bp, Artificial Sequence
  Description of Artificial Sequence Synthetic probe
caggaggcanagaaattacaggcc

SEQ ID NO: 9 - DNA, 21 bp, Artificial Sequence
  Description of Artificial Sequence Synthetic primer
gtccagctgtgcaagagaata

SEQ ID NO: 10 - DNA, 21 bp, Artificial Sequence
  Description of Artificial Sequence Synthetic primer
tacagctttcagtgcaaagga

SEQ ID NO: 11 - DNA, 23 bp, Artificial Sequence
  Description of Artificial Sequence Synthetic probe
cgctctccgnagaagctcttcct

SEQ ID NO: 12 - DNA, 21 bp, Artificial Sequence
  Description of Artificial Sequence Synthetic primer
gtccagctgtgcaagagaata

SEQ ID NO: 13 - DNA, 21 bp, Artificial Sequence
  Description of Artificial Sequence Synthetic primer
tacagctttcagtgcaaagga

SEQ ID NO: 14 - DNA, 23 bp, Artificial Sequence
  Description of Artificial Sequence Synthetic probe
cgctctccgnagaagctcttcct

SEQ ID NO: 15 - DNA, 21 bp, Artificial Sequence
  Description of Artificial Sequence Synthetic primer
gtccagctgtgcaagagaata

SEQ ID NO: 16 - DNA, 21 bp, Artificial Sequence
  Description of Artificial Sequence Synthetic primer
tacagctttcagtgcaaagga

SEQ ID NO: 17 - DNA, 23 bp, Artificial Sequence
  Description of Artificial Sequence Synthetic probe
cgctctccgnagaagctcttcct

SEQ ID NO: 18 - DNA, 30 bp, Artificial Sequence
  Description of Artificial Sequence Synthetic primer
aaaaacagctgcgatcagaggcgcaagatg

SEQ ID NO: 19 - DNA, 25 bp, Artificial Sequence
  Description of Artificial Sequence Synthetic primer
gctgatctctgagtttcgcattctg

SEQ ID NO: 20 - DNA, 615 bp, Homo sapiens
atgcaatcatatgcttctgctatgttaagcgtactcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctgtag

SEQ ID NO: 21 - DNA, 48 bp, Artificial Sequence
  Description of Artificial Sequence Synthetic primer
aaaaacagctggtgaagcgacccatgaacgcgtgtggtctcgcgatca

SEQ ID NO: 22 - DNA, 37 bp, Artificial Sequence
  Description of Artificial Sequence Synthetic primer
tgatcgcgagaccacacgcgttcatgggtcgcttcac

SEQ ID NO: 23 - DNA, 30 bp, Artificial Sequence; identical to SEQ ID NO: 18
  Description of Artificial Sequence Synthetic primer

SEQ ID NO: 24 - DNA, 25 bp, Artificial Sequence
  Description of Artificial Sequence Synthetic primer
gctgatctctgagtttcgcattctg

SEQ ID NO: 25 - DNA, 615 bp, Homo sapiens; identical to SEQ ID NO: 20

SEQ ID NO: 26 - DNA, 53 bp, Artificial Sequence
  Description of Artificial Sequence Synthetic primer
aaaaacagctgggccatgcacagagagaaatacgtatcgacctcgtcggaagg

SEQ ID NO: 27 - DNA, 42 bp, Artificial Sequence
  Description of Artificial Sequence Synthetic primer
ccttccgacgaggtcgatacgtatttctctctgtgcatggcc

SEQ ID NO: 28 - DNA, 54 bp, Artificial Sequence
  Description of Artificial Sequence Synthetic primer
aaaaacagctgaagctcttccttcctttgcacgtaaaggcaacgtccaggatag

SEQ ID NO: 29 - DNA, 43 bp, Artificial Sequence
  Description of Artificial Sequence Synthetic primer
ctatcctggacgttgcctttacgtgcaaaggaaggaagagctt

SEQ ID NO: 30 - DNA, 23 bp, Artificial Sequence
  Description of Artificial Sequence Synthetic primer
tgatctctgagtttcgcattctg

SEQ ID NO: 31 - DNA, 26 bp, Artificial Sequence
  Description of Artificial Sequence Synthetic primer
aaaaaancgatcagaggcgcaagatg

(19) United States
(12) Patent Application Publication
(10) Pub. No.: US 2010/0279295 A1
(43) Pub. Date: Nov. 4, 2010

(54) USE OF THERMOSTABLE ENDONUCLEASES FOR GENERATING REPORTER MOLECULES

(75) Inventors: Margaret Ann Roy, San Diego, CA (US); Paul Andrew Oeth, San Diego, CA (US)

Correspondence Address: GRANT ANDERSON LLP;C/O PORTFOLIOIP, PO BOX 52050, MINNEAPOLIS, MN, 55402, US

(73) Assignee: SEQUENOM, INC., San Diego, CA (US)

(21) Appl. No.: 12/726,246

(22) Filed: Mar. 17, 2010

Related U.S. Application Data
(60) Provisional application No. 61/161,385, filed on Mar. 18, 2009.

(51) Int. Cl. C12Q 1/68; C12P 19/34

(52) U.S. Cl. 435/6; 435/91.2

(57) ABSTRACT

Provided are compositions and methods for amplifying, capturing and/or detecting target nucleic acids using cleavable oligonucleotides.

### RELATED PATENT APPLICATION

This application claims the benefit of U.S. provisional patent application No. 61/161,385 filed on Mar. 18, 2009, entitled USE OF THERMOSTABLE ENDONUCLEASES FOR GENERATING REPORTER MOLECULES, naming Margaret Ann Roy and Paul Andrew Oeth as inventors and designated by Attorney Docket No. SEQ-6025-PV. The entire content of the foregoing patent application is incorporated herein by reference, including, without limitation, all text, tables and drawings

### FIELD

The technology relates in part to compositions and methods for amplifying and/or detecting nucleic acids.

### BACKGROUND

Amplification of nucleic is widely utilized in many laboratory techniques and clinical or diagnostic procedures. With the addition of multiplexed reactions and manual or automated high throughput techniques and apparatus, the ability exits to rapidly amplify and detect large numbers of target nucleic acid sequences, such as microarray based genotyping or whole genome sequencing, for example.

Amplification of nucleic acids by thermocycling or isothermal procedures allows rapid, specific amplification of target nucleic acids. Undesired amplification products, referred to as “amplification artifacts,” can arise due to the extension of improperly annealed nucleic acids by a polymerase, for example, as the temperature in the reaction vessel increases and the polymerase becomes increasingly active. Improvements to reaction techniques and conditions (e.g., hot start PCR techniques) have minimized amplification artifacts (e.g., such as “primer-dimer” or incorrect or non-specific annealing of amplification oligonucleotides). Hot start amplification techniques often involve partitioning or inhibiting reaction components until a certain temperature is reached, thereby allowing contact, mixing and activation of components and extension of oligonucleotides annealed to a specific target nucleic acid.

### SUMMARY

In some embodiments, provided are methods for amplifying a target nucleic acid, or portion thereof, in a nucleic acid composition, which comprise: (a) contacting, under hybridization conditions, a nucleic acid composition with two oligonucleotide species, where each oligonucleotide species comprises: (i) a nucleotide subsequence complementary to the target nucleic acid, (ii) a non-terminal and non-functional portion of a first endonuclease cleavage site, where the portion of the first endonuclease cleavage site may form a functional first endonuclease cleavage site when the oligonucleotide species is hybridized to the target nucleic acid, and (iii) a blocking moiety at the 3′ end of the oligonucleotide species; (b) cleaving the first functional cleavage site with a first endonuclease under cleavage conditions, thereby generating an extendable primer and a fragment comprising the blocking moiety; and (c) extending the extendable primer under amplification conditions, whereby the target nucleic acid, or portion thereof, is amplified.

In some embodiments, the fragment comprising the blocking moiety may comprise a detectable feature. In certain embodiments, the method can further comprise detecting the detectable feature. In some embodiments the fragment comprising the blocking moiety can comprise a capture agent. In some embodiments, the blocking moiety of a first oligonucleotide species may be different than the blocking moiety of a second oligonucleotide species. In certain embodiments the blocking moiety of each oligonucleotide species independently may be selected from the group consisting of biotin, avidin, streptavidin and a detectable label. In some embodiments, steps where (a), (b) and (c) can be performed in the same reaction environment and/or are performed contemporaneously.

In certain embodiments, one of the oligonucleotide species comprises a 5′ region, where the 5′ region may comprise: (i) a nucleotide subsequence not complementary to the target nucleic acid, (ii) a non-functional portion of a second endonuclease cleavage site, whereby the non-functional portion of the second endonuclease cleavage site is converted into a functional second endonuclease cleavage site under the amplification conditions, and (iii) a detectable feature. In some embodiments, cleaving the functional second endonuclease cleavage site with a second endonuclease under cleavage conditions, thereby generating a fragment comprising the detectable feature. In certain embodiments, the cleaving may generate two or more fragments comprising distinguishable detectable features. In some embodiments, the method further comprises detecting one or more of the detectable features of one or more of the fragments. In certain embodiments, one or more of the fragments may comprise a capture agent. In some embodiments, the cleaving with the second endonuclease can be performed in the same reaction environment as (a), (b) and (c), and/or can be performed contemporaneously with (a), (b) and (c).

In certain embodiments, also provided are methods for detecting a target nucleic acid in a nucleic acid composition, which comprise: (a) contacting, under hybridization conditions, a nucleic acid composition with two oligonucleotide species, where each oligonucleotide species may comprise: (i) a nucleotide subsequence complementary to the target nucleic acid, (ii) a non-terminal and non-functional portion of a first endonuclease cleavage site, where the portion of the first endonuclease cleavage site forms a functional first endonuclease cleavage site when the oligonucleotide species is hybridized to the target nucleic acid, (iii) a detectable feature, and (iv) a blocking moiety at the 3′ end of the oligonucleotide species; (b) contacting, under cleavage conditions, the nucleic acid composition with a first endonuclease, where the first endonuclease can cleave the functional first endonuclease cleavage site when target nucleic acid is present, thereby generating and releasing a cleavage product having the detectable feature; and (c) detecting the presence or absence of the cleavage product having the detectable feature, whereby the presence or absence of the target nucleic acid can be detected based on detecting the presence or absence of the cleavage product with the detectable feature.

In some embodiments, steps (a) and (b) can be performed in the same reaction environment. In certain embodiments, steps (a) and (b) may be performed contemporaneously. In some embodiments, the cleaving in (b) can generate two or more cleavage products comprising distinguishable detectable features. In certain embodiments, one or more of the detectable features of one or more of the cleavage products can be detected. In some embodiments, one or more of the cleavage products may comprise a capture agent.

In certain embodiments, also provided are methods for detecting a target nucleic acid in a nucleic acid composition, which comprise: (a) contacting, under hybridization conditions, a nucleic acid composition with two oligonucleotide species, where each oligonucleotide species may comprise: (i) a nucleotide subsequence complementary to the target nucleic acid, (ii) a non-terminal and non-functional portion of a first endonuclease cleavage site, where the portion of the first endonuclease cleavage site can form a functional first endonuclease cleavage site when the oligonucleotide species is hybridized to the target nucleic acid, iii) a detectable feature, and (iv) a blocking moiety at the 3′ end of the oligonucleotide species, and where one of the oligonucleotide species can comprise a non-functional portion of a second endonuclease cleavage site; (b) cleaving the first functional cleavage site with a first endonuclease under cleavage conditions, thereby generating an extendable primer; (c) extending the extendable primer under amplification conditions, whereby the non-functional portion of the second endonuclease cleavage site can be converted into a functional second endonuclease cleavage site under the amplification conditions; (d) cleaving the functional second endonuclease cleavage site with a second endonuclease under cleavage conditions, thereby generating a cleavage product having the detectable feature; and (e) detecting the presence or absence of the cleavage product having the detectable feature, whereby the presence or absence of the target nucleic acid can be detected based on detecting the presence or absence of the cleavage product with the detectable feature.

In some embodiments, steps (a), (b), (c) and (d) can be performed in the same reaction environment, and in certain embodiments can be performed contemporaneously. In certain embodiments, the cleaving in (b) can generate two or more cleavage products comprising distinguishable detectable features. In some embodiments, one or more of the detectable features of one or more of the cleavage products can be detected. In certain embodiments, one or more of the cleavage products may comprise a capture agent.

In certain embodiments, provided are methods for amplifying a target nucleic acid, or portion thereof, in a nucleic acid composition, which comprise: (a) contacting, under hybridization conditions, a nucleic acid composition with an oligonucleotide and forward and reverse polynucleotide primers, where: (i) the oligonucleotide may comprise a nucleotide subsequence complementary to the target nucleic acid, (ii) the oligonucleotide may comprise a non-terminal and non-functional portion of a first endonuclease cleavage site, where the portion of the first endonuclease cleavage site can form a functional first endonuclease cleavage site when the oligonucleotide species is hybridized to the target nucleic acid, (iii) the oligonucleotide may comprise a blocking moiety at the 3′ end of the oligonucleotide species, (iv) one of the polynucleotide primers hybridizes to the target nucleic acid 5′ of the oligonucleotide; (b) cleaving the first functional cleavage site with a first endonuclease under cleavage conditions, thereby generating cleavage products; and (c) extending the polynucleotide primers under amplification conditions, whereby the target nucleic acid, or portion thereof, is amplified.

In certain embodiments, the oligonucleotide can block extension of the polynucleotide primer until the first functional cleavage site is cleaved by the first endonuclease. In some embodiments, steps (a), (b), (c) and (d) can be performed in the same reaction environment, and in certain embodiments can be performed contemporaneously. In some embodiments, one or more cleavage products may include a detectable feature. In certain embodiments, the method further comprises detecting the detectable feature in the one or more cleavage products. In some embodiments, one or more cleavage products include a capture agent.

In some embodiments, provided are methods for determining the presence or absence of a target nucleic acid in a nucleic acid composition, which comprise: (a) contacting, under hybridization conditions, a nucleic acid composition with an oligonucleotide comprising: (i) a nucleotide subsequence complementary to the target nucleic acid, (ii) a non-terminal and non-functional portion of an endonuclease cleavage site, where the portion of the endonuclease cleavage site can form a functional endonuclease cleavage site when the oligonucleotide is hybridized to the target nucleic acid, (iii) a blocking moiety at the 3′ end of the oligonucleotide, and (iv) a detectable feature; (b) contacting the nucleic acid composition with an endonuclease capable of cleaving the cleavage site under cleavage conditions, thereby generating oligonucleotide fragments having the detectable feature when the target nucleic acid is present; and (c) detecting the presence or absence of the oligonucleotide fragments having the detectable feature, whereby the presence or absence of the target nucleic acid can be determined based upon detecting the presence or absence of the oligonucleotide fragments. In some embodiments, steps (a), (b), (c) and (d) can be performed in the same reaction environment, and in certain embodiments can be performed contemporaneously. In some embodiments, the cleaving in (b) can generate two or more oligonucleotide fragments comprising distinguishable detectable features. In certain embodiments, one or more of the detectable features of one or more of the oligonucleotide fragments can be detected. In some embodiments, one or more of the oligonucleotide fragments can comprise a capture agent.

In some embodiments, also provided are methods for determining the presence or absence of a target nucleic acid in a nucleic acid composition, which comprise: (a) contacting, under hybridization conditions, a nucleic acid composition with an oligonucleotide comprising: (i) a nucleotide subsequence complementary to the target nucleic acid, (ii) a non-terminal and non-functional portion of an endonuclease cleavage site, where the portion of the endonuclease cleavage site can form a functional endonuclease cleavage site when the oligonucleotide is hybridized to the target nucleic acid, (iii) a blocking moiety at the 3′ end of the oligonucleotide, and (iv) a detectable feature; (b) contacting the nucleic acid composition with an endonuclease capable of cleaving the cleavage site under cleavage conditions, thereby generating oligonucleotide fragments having the detectable feature when the target nucleic acid is present; (c) contacting the nucleic acid composition with forward and reverse primer polynucleotides under extension conditions; and (d) detecting the presence or absence of the oligonucleotide fragments having the detectable feature, whereby the presence or absence of the target nucleic acid can be determined based upon detecting the presence or absence of the oligonucleotide fragments. In some embodiments the nucleic acid can be contacted with two or more oligonucleotide species.

In certain embodiments, steps (a), (b), (c) and (d) can be performed in the same reaction environment, and in certain embodiments can be performed contemporaneously. In some embodiments, the cleaving in (b) can generate two or more oligonucleotide fragments comprising distinguishable detectable features. In certain embodiments, one or more of the detectable features of one or more of the oligonucleotide fragments can be detected. In some embodiments, one or more of the oligonucleotide fragments can comprise a capture agent.

In some embodiments, provided are methods for amplifying a target nucleic acid, or portion thereof, in a nucleic acid composition, which comprise: (a) contacting, under hybridization conditions, a nucleic acid composition with an oligonucleotide and a primer polynucleotide, where the oligonucleotide comprises: (i) a nucleotide subsequence complementary to the target nucleic acid, and (ii) a non-terminal and non-functional portion of a first endonuclease cleavage site; and (b) extending the oligonucleotide under amplification conditions, thereby generating an extended oligonucleotide, where the primer polynucleotide hybridizes to the extended oligonucleotide and is extended under the amplification conditions, thereby yielding a double-stranded amplification product that comprises a functional first endonuclease cleavage site, whereby the target nucleic acid, or portion thereof, is amplified. In some embodiments, the method can further comprise (c) cleaving the first functional cleavage site with a first endonuclease under cleavage conditions, thereby generating a double-stranded cleavage product.

In certain embodiments, the double-stranded cleavage product comprises a detectable feature.

In some embodiments, the method further comprises detecting the detectable feature. In some embodiments, the double-stranded cleavage product comprises a capture agent. In certain embodiments steps (a) and (b) can performed in the same reaction environment, and in some embodiments can be performed contemporaneously.

In some embodiments, the method may further comprise (c) cleaving the first functional cleavage site with a first endonuclease under cleavage conditions, thereby generating a single-stranded cleavage product. In some embodiments, the single-stranded cleavage product may comprise a detectable feature. In certain embodiments, the method can further comprise detecting the detectable feature. In some embodiments, the single-stranded cleavage product may comprise a capture agent.

In certain embodiments, provided are methods for detecting the presence or absence of a target nucleic acid in a nucleic acid composition, which comprise: (a) contacting, under hybridization conditions, a nucleic acid composition with an oligonucleotide and a primer polynucleotide, where the oligonucleotide comprises: (i) a nucleotide subsequence complementary to the target nucleic acid, (ii) a non-terminal and non-functional portion of a first endonuclease cleavage site, and (iii) a detectable feature; and (b) exposing the nucleic acid composition to amplification conditions, where (i) the oligonucleotide can be extended when the target nucleic acid is present, and (ii) the primer polynucleotide hybridizes to the extended oligonucleotide and can be extended under the amplification conditions, thereby yielding a double-stranded amplification product that comprises a functional first endonuclease cleavage site; (c) contacting the nucleic acid composition with a first endonuclease that cleaves the functional first endonuclease cleavage site, thereby generating a cleavage product comprising the detectable feature; and (d) detecting the presence or absence of the cleavage product comprising the detectable feature, whereby the presence or absence of the target nucleic acid can be detected based on the presence or absence of the cleavage product comprising the detectable feature.

In certain embodiments, steps (a), (b), (c) can be performed in the same reaction environment, and in certain embodiments can be performed contemporaneously. In some embodiments, the cleaving in (c) can generate two or more cleavage products comprising distinguishable detectable features. In certain embodiments, one or more of the detectable features of one or more of the cleavage products can be detected. In some embodiments, one or more of the cleavage products can comprise a capture agent.

In certain embodiments, provided are methods for amplifying a target nucleic acid, or portion thereof, in a nucleic acid composition, which comprise: (a) providing an oligonucleotide and a polynucleotide, or providing an oligonucleotide that includes a 3′ portion, under hybridization conditions, where: (i) the oligonucleotide comprises a nucleotide subsequence complementary to the target nucleic acid, (ii) the polynucleotide comprises a polynucleotide subsequence complementary to (“complementary polynucleotide sequence”) and hybridized to a complementary subsequence of the oligonucleotide, (iii) the 3′ portion of the oligonucleotide comprises a polynucleotide subsequence complementary to (“complementary polynucleotide sequence”) and hybridized to a 5′ complementary subsequence of the oligonucleotide, and (iv) the complementary subsequence of the oligonucleotide and the complementary polynucleotide sequence comprise a functional first endonuclease cleavage site; (b) cleaving the first functional cleavage site with a first endonuclease under cleavage conditions, thereby generating an extendable primer oligonucleotide; (c) contacting the nucleic acid composition with the extendable primer oligonucleotide; (d) extending the extendable primer oligonucleotide under amplification conditions in the presence of a primer nucleic acid, where (i) an extended primer oligonucleotide is generated, and (ii) the primer nucleic acid hybridizes to the extended primer oligonucleotide and is extended, whereby the target nucleic acid, or portion thereof, is amplified.

In some embodiments, the oligonucleotide can comprise a non-functional portion of a second endonuclease cleavage site, and a double-stranded amplification product comprising a functional second endonuclease cleavage site can be generated under the amplification conditions. In certain embodiments, the method may further comprise (e) cleaving the functional second endonuclease cleavage site with a second endonuclease, thereby generating a cleavage product. In some embodiments, the cleavage product is double-stranded (e.g., the endonuclease cleaves both strands of the double-stranded amplification product). In certain embodiments, the cleavage product is single-stranded (e.g., the endonuclease cleaves one strand of the double-stranded amplification product). In some embodiments, the cleaving generates two or more cleavage products comprising distinguishable detectable features. In certain embodiments, one or more of the detectable features of one or more of the cleavage products can be detected. In some embodiments, one or more of the cleavage products can comprise a capture agent. In some embodiments, the oligonucleotide and the polynucleotide can comprise the same or a different blocking moiety. In certain embodiments, steps (a), (b), (c) and (d), or (a), (b), (c), (d) and (e), can be performed in the same reaction environment. In some embodiments, steps (a), (b), (c) and (d), or (a), (b), (c), (d) and (e), can be performed contemporaneously. In certain embodiments, the oligonucleotide that includes a 3′ portion can form a stem-loop structure.

In some embodiments, also provided are methods for detecting a target nucleic acid in a nucleic acid composition, which comprise: (a) providing an oligonucleotide and a polynucleotide, or providing an oligonucleotide that includes a 3′ portion, under hybridization conditions, where: (i) the oligonucleotide can comprise a nucleotide subsequence complementary to the target nucleic acid, (ii) the polynucleotide comprises a polynucleotide subsequence complementary to (“complementary polynucleotide sequence”) and hybridized to a complementary subsequence of the oligonucleotide, (iii) the 3′ portion of the oligonucleotide can comprise a polynucleotide subsequence complementary to (“complementary polynucleotide sequence”) and hybridized to a 5′ complementary subsequence of the oligonucleotide, (iv) the complementary subsequence of the oligonucleotide and the complementary polynucleotide sequence comprise a functional first endonuclease cleavage site, (v) the oligonucleotide comprises a non-functional portion of a second endonuclease cleavage site, and (vi) the oligonucleotide can comprise a detectable feature; (b) providing a first endonuclease under cleavage conditions, where the first endonuclease cleaves the first endonuclease cleavage site, thereby generating an extendable primer oligonucleotide; (c) contacting the nucleic acid composition with the extendable primer oligonucleotide; (d) exposing the nucleic acid composition to amplification conditions and a primer nucleic acid, where: (i) the extendable primer oligonucleotide can be extended when the target nucleic acid is present, thereby generating an extended primer oligonucleotide, and (ii) the primer nucleic acid hybridizes to the extended primer oligonucleotide and is extended, thereby generating a double-stranded amplification product comprising a functional second endonuclease cleavage site; (e) contacting the nucleic acid composition with a second endonuclease under cleavage conditions, where the second endonuclease cleaves double-stranded amplification product comprising the functional second endonuclease cleavage site, thereby generating a cleavage product comprising the detectable feature; and (f) detecting the presence or absence of the cleavage product comprising the detectable feature, whereby the presence or absence of the target nucleic acid can be detected based on detecting the presence or absence of the cleavage product comprising the detectable feature.

In some embodiments, steps (a), (b), (c), (d) and (e) are performed in the same reaction environment, and in certain embodiments are performed contemporaneously. In some embodiments, the cleavage product is double-stranded (e.g., the endonuclease cleaves both strands of the double-stranded amplification product). In certain embodiments, the cleavage product is single-stranded (e.g., the endonuclease cleaves one strand of the double-stranded amplification product). In some embodiments, the cleaving generates two or more cleavage products comprising distinguishable detectable features. In certain embodiments, one or more of the detectable features of one or more of the cleavage products can be detected. In some embodiments, one or more of the cleavage products can comprise a capture agent.

In certain embodiments, amplification and/or extension conditions include a nucleic acid polymerase. In some embodiments, the nucleic acid polymerase is a DNA polymerase, and in certain embodiments, the nucleic acid polymerase is a RNA polymerase. In some embodiments, the polymerase is a trans-lesion synthesizing polymerase, and sometimes the the polymerase is a trans-lesion Y-family polymerase (e.g., Sulfolobus DNA Polymerase IV). In certain embodiments, the polymerase is capable of synthesizing DNA across one or more DNA template lesions, and sometimes the one or more lesions include one or more abasic sites. In some embodiments, the polymerase is selected from Taq DNA Polymerase; Q-Bio™ Taq DNA Polymerase; SurePrime™ Polymerase; Arrow™ Taq DNA Polymerase; JumpStart Taq™; 9° N™m DNA polymerase; Deep Vent_{R}™ (exo-) DNA polymerase; Tth DNA polymerase; antibody-mediated polymerases; polymerases for thermostable amplification; native or modified RNA polymerases, and functional fragments thereof, native or modified DNA polymerases and functional fragments thereof, the like and combinations thereof.

In some embodiments, provided are methods for determining the presence or absence of a target nucleic acid in a nucleic acid composition, which comprise: (a) contacting the nucleic acid composition with an oligonucleotide, under hybridization conditions, where the oligonucleotide comprises: (i) the oligonucleotide comprises a terminal 5′ region, an internal 5′ region, an internal 3′ region and a terminal 3′ region, (ii) the oligonucleotide comprises a blocking moiety at the 3′ terminus, and (iii) the terminal 5′ region and the terminal 3′ region are substantially complementary to, and can hybridize to, the target nucleic acid, (iv) the internal 5′ region and the internal 3′ region are not complementary to the target nucleic acid, (v) the internal 5′ region is substantially complementary to the internal 3′ region and hybridize to one another to form an internal stem-loop structure when the terminal 5′ region and the terminal 3′ region are hybridized to the target nucleic acid, (vi) the internal 5′ region and the internal 3′ region do not hybridize to one another when the terminal 5′ region and the terminal 3′ region are not hybridized to the target nucleic acid, and (vii) the stem-loop structure comprises an endonuclease cleavage site; (b) contacting the nucleic acid composition with an endonuclease capable of cleaving the cleavage site, whereby a stem-loop structure cleavage product may be generated if the target nucleic acid is present in the nucleic acid composition; and (c) detecting the presence or absence of the cleavage product, whereby the presence or absence of the target nucleic acid can be determined based upon detecting the presence or absence of the cleavage product. In some embodiments, the cleavage product comprises a detectable feature. In certain embodiments, the cleavage product comprises a capture agent. In some embodiments, steps (a) and (b) can be performed in the same reaction environment, and in certain embodiments are performed contemporaneously.

In certain embodiments, provided are methods for determining the presence or absence of a target nucleic acid in a nucleic acid composition, which comprise: (a) contacting the nucleic acid composition with a first oligonucleotide and a second oligonucleotide under hybridization conditions, where: (i) the first oligonucleotide and the second oligonucleotide each comprise a 5′ region, a 3′ region and a blocking moiety at the 3′ terminus, (ii) the 5′ region of the first oligonucleotide and the 3′ region of the second oligonucleotide are substantially complementary to, and can hybridize to, the target nucleic acid, (iii) the 3′ region of the first oligonucleotide and the 5′ region of the second oligonucleotide are not complementary to the target nucleic acid, (iv) the 3′ region of the first oligonucleotide is substantially complementary to the 5′ region of the second oligonucleotide are can hybridize to one another to form a stem structure when the 5′ region of the first oligonucleotide and the 3′ region of the second oligonucleotide are hybridized to the target nucleic acid, (v) the 3′ region of the first oligonucleotide and the 5′ region of the second oligonucleotide do not hybridize to one another when the 5′ region of the first oligonucleotide and the 3′ region of the second oligonucleotide are not hybridized to the target nucleic acid, and (vi) the stem structure comprises an endonuclease cleavage site; (b) contacting the nucleic acid composition with an endonuclease capable of cleaving the cleavage site, whereby a stem structure cleavage product can be generated if the target nucleic acid is present in the nucleic acid composition; and (c) detecting the presence or absence of the cleavage product, whereby the presence or absence of the target nucleic acid can be determined based upon detecting the presence or absence of the cleavage product. In some embodiments, the cleavage product comprises a detectable feature. In certain embodiments, the cleavage product comprises a capture agent. In some embodiments, steps (a) and (b) can be performed in the same reaction environment, and in certain embodiments can be performed contemporaneously.

In some embodiments, the capture agent can be selected from the group consisting of biotin, avidin and streptavidin. In certain embodiments, the endonuclease can be thermostable. In some embodiments, the endonuclease loses less than about 50% of its maximum activity under the amplification conditions. In certain embodiments, the endonuclease cleavage site can include an abasic site. In some embodiments, the endonuclease may be an AP endonuclease. In certain embodiments, the AP endonuclease can be selected from Tth endonuclease IV, and the AP endonucleases from Thermotogoa maritime, Thermoplasm volacanium and lactobacillus plantarum.

In certain embodiments, the endonuclease can be a restriction endonuclease. In some embodiments, the restriction endonuclease can have double-stranded cleavage activity. In certain embodiments, the restriction endonuclease can have single-stranded cleavage activity (e.g., nicking enzyme). In some embodiments, the restriction endonuclease can be selected from Acl I, Apa LI, Ape KI, Bam HI, Bam HI-HF, Bcl I, Bgl II, Blp I, Bsa AI, Bsa XI, Bsi HKAI, Bso BI, Bsr FI, Bst BI, Bst EII, Bst NI, Bst UI, Bst Z17I, Bts CI, Cvi QI, Hpa I, Kpn I, Mwo I, Nci I, Pae R7I, Pho I, Ppu MI, Pvu II, Sfi I, Sfo I, Sml I, Tti I, Tsp 509I, Tsp MI, Tsp RI, and Zra I.

In certain embodiments, the endonuclease may cleave DNA. In some embodiments, the endonuclease does not cleave RNA. In certain embodiments, the endonuclease is not an RNase. In some embodiments, the oligonucleotide can comprise one or more abasic sites. In certain embodiments, the oligonucleotide can comprise one or more non-cleavable bases. In some embodiments, the one or more non-cleavable bases can be in a cleavage site, the restriction endonuclease may have double-stranded cleavage activity, and the restriction endonuclease may cleave only one strand of the cleavage site.

In certain embodiments, the detectable feature may be selected from the group consisting of mass (e.g., inherent mass of nucleic acid, inherence mass of cleavage product), length, nucleotide sequence, optical property, electrical property, magnetic property, chemical property and time or speed through an opening in a matrix material or other material (e.g., nanopore). In some embodiments, the detectable feature can be mass. In certain embodiments, the mass may be detected by mass spectrometry. In some embodiments, the mass spectrometry can be selected from the group consisting of Matrix-Assisted Laser Desorption/lonization Time-of-Flight (MALDI-TOF) Mass Spectrometry (MS), Laser Desorption Mass Spectrometry (LDMS), Electrospray (ES) MS, Ion Cyclotron Resonance (ICR) MS, and Fourier Transform MS. In certain embodiments, the mass spectrometry comprises ionizing and volatizing nucleic acid.

In some embodiments, the detectable feature can be a signal detected from a detectable label. In certain embodiments, the signal may be selected from the group consisting of fluorescence, luminescence, ultraviolet light, infrared light, visible wavelength light, light scattering, polarized light, radiation and isotope radiation. In some embodiments, the amplification conditions may comprise a polymerase having strand displacement activity. In certain embodiments, the blocking moiety can be a 3′ terminal moiety selected from the group consisting of phosphate, amino, thiol, acetyl, biotin, cholesteryl, tetraethyleneglycol (TEG), biotin-TEG, cholesteryl-TEG, one or more inverted nucleotides, inverted deoxythymidine, digoxigenin, and 1,3-propanediol (C3 spacer).

In some embodiments, the loop in the stem-loop structure can comprise nucleotides. In certain embodiments, the loop in the stem-loop structure can comprise a non-nucleotide linker. In some embodiments, the stem in the stem-loop structure can be partially single-stranded. In certain embodiments, the stem in the stem-loop structure can be double-stranded. In some embodiments, the stem-loop structure or stem structure can comprise one or both members of a signal molecule pair, where the signal molecule pair members can be separated by the endonuclease cleavage site. In certain embodiments, the signal molecule pair members are fluorophore and quencher molecules. In some embodiments, the signal molecule pair members are fluorophore molecules suitable for fluorescence resonance energy transfer (FRET). In certain embodiments, the first endonuclease is different than the second endonuclease.

In certain embodiments, provided are compositions of matter comprising a blocked oligonucleotide that include: (i) a non-terminal abasic site, (ii) a blocking moiety at the 3′ terminus, and (iii) a detectable feature.

In some embodiments, provided are compositions of matter that comprise two oligonucleotide species, where each oligonucleotide species includes: (i) a nucleotide subsequence complementary to a target nucleic acid, (ii) a non-terminal and non-functional portion of a first endonuclease cleavage site, where the portion of the first endonuclease cleavage site can form a functional first endonuclease cleavage site when the oligonucleotide species is hybridized to the target nucleic acid, and (iii) a blocking moiety at the 3′ end of the oligonucleotide species. In some embodiments, one of the oligonucleotide species can comprise a 5′ region that includes: (i) a nucleotide subsequence not complementary to the target nucleic acid, (ii) a non-functional portion of a second endonuclease cleavage site, whereby the non-functional portion of the second endonuclease cleavage site is converted into a functional second endonuclease cleavage site under amplification conditions, and (iii) a detectable feature.

In some embodiments, provided are compositions of matter that comprise an oligonucleotide and a polynucleotide hybridized to one another, where: (i) the oligonucleotide can comprise a nucleotide subsequence complementary to a target nucleic acid, (ii) the polynucleotide can comprise a polynucleotide subsequence complementary to (“complementary polynucleotide sequence”) and hybridized to a complementary subsequence of the oligonucleotide, and (iii) the complementary subsequence of the oligonucleotide and the complementary polynucleotide sequence may comprise a functional first endonuclease cleavage site. In some embodiments, the oligonucleotide and the polynucleotide each comprise a blocking moiety at the 3′ terminus.

In certain embodiments, provided are compositions of matter that comprise an oligonucleotide and a polynucleotide hybridized to one another, where: (i) the oligonucleotide can comprise a nucleotide subsequence complementary to a target nucleic acid, (ii) the polynucleotide can comprise a polynucleotide subsequence complementary to (“complementary polynucleotide sequence”) and hybridized to a complementary subsequence of the oligonucleotide, (iii) the complementary subsequence of the oligonucleotide and the complementary polynucleotide sequence may comprise a functional first endonuclease cleavage site, and (iv) the oligonucleotide comprises a non-functional portion of a second endonuclease cleavage site. In certain embodiments, the oligonucleotide and the polynucleotide each comprise a blocking moiety at the 3′ terminus.

In some embodiments, provided are compositions of matter that comprise an oligonucleotide, where: (i) the oligonucleotide may comprise a nucleotide subsequence complementary to the target nucleic acid, (ii) the oligonucleotide can comprise a 3′ portion that comprises a polynucleotide subsequence complementary to (“complementary polynucleotide sequence”) and hybridized to a 5′ complementary subsequence of the oligonucleotide, thereby forming a stem-loop structure, and (iii) the complementary subsequence of the oligonucleotide and the complementary polynucleotide sequence can comprise a functional first endonuclease cleavage site. In some embodiments, the oligonucleotide and the polynucleotide each comprise a blocking moiety at the 3′ terminus.

In certain embodiments, provided are compositions of matter that comprise an oligonucleotide, where: (i) the oligonucleotide can comprise a nucleotide subsequence complementary to the target nucleic acid, (ii) the oligonucleotide can comprise a 3′ portion that comprises a polynucleotide subsequence complementary to (“complementary polynucleotide sequence”) and hybridized to a 5′ complementary subsequence of the oligonucleotide, thereby forming a stem-loop structure, (iii) the complementary subsequence of the oligonucleotide and the complementary polynucleotide sequence may comprise a functional first endonuclease cleavage site, and (iv) the oligonucleotide can comprise a non-functional portion of a second endonuclease cleavage site. In some embodiments, the oligonucleotide and the polynucleotide each comprise a blocking moiety at the 3′ terminus.

In some embodiments, provided are compositions of matter that comprise an oligonucleotide, where: (i) the oligonucleotide may comprise a terminal 5′ region, an internal 5′ region, an internal 3′ region and a terminal 3′ region, (ii) the oligonucleotide can comprise a blocking moiety at the 3′ terminus, and (iii) the terminal 5′ region and the terminal 3′ region are substantially complementary to, and can hybridize to, a target nucleic acid, (iv) the internal 5′ region and the internal 3′ region are not complementary to the target nucleic acid, (v) the internal 5′ region is substantially complementary to the internal 3′ region and hybridize to one another to form an internal stem-loop structure when the terminal 5′ region and the terminal 3′ region are hybridized to the target nucleic acid, (vi) the internal 5′ region and the internal 3′ region do not hybridize to one another when the terminal 5′ region and the terminal 3′ region are not hybridized to the target nucleic acid, and (vii) the stem-loop structure can comprise an endonuclease cleavage site.

In certain embodiments, provided are compositions of matter that comprise a first oligonucleotide and a second oligonucleotide, where: (i) the first oligonucleotide and the second oligonucleotide each comprise a 5′ region, a 3′ region and a blocking moiety at the 3′ terminus, (ii) the 5′ region of the first oligonucleotide and the 3′ region of the second oligonucleotide are substantially complementary to, and can hybridize to, the target nucleic acid, (iii) the 3′ region of the first oligonucleotide and the 5′ region of the second oligonucleotide are not complementary to the target nucleic acid, (iv) the 3′ region of the first oligonucleotide can be substantially complementary to the 5′ region of the second oligonucleotide are can hybridize to one another to form a stem structure when the 5′ region of the first oligonucleotide and the 3′ region of the second oligonucleotide are hybridized to the target nucleic acid, (v) the 3′ region of the first oligonucleotide and the 5′ region of the second oligonucleotide do not hybridize to one another when the 5′ region of the first oligonucleotide and the 3′ region of the second oligonucleotide are not hybridized to the target nucleic acid, and (vi) the stem structure can comprise an endonuclease cleavage site.

Certain embodiments are described further in the following description, claims and drawings.

### BRIEF DESCRIPTION OF THE DRAWINGS

The drawings illustrate certain non-limiting embodiments of the technology. For clarity and ease of illustration, drawings are not necessarily to scale, and in some instances, various elements may be shown exaggerated or enlarged to facilitate an understanding of particular embodiments.

FIG. 1 is a schematic representation of a method for amplifying and detecting a target nucleic acid using a 3′ phosphate blocked, abasic oligonucleotide species composition (e.g., “probe” oligonucleotide) in conjunction with unmodified forward and reverse oligonucleotide species (e.g., forward and reverse “primers”, for example). The reverse oligonucleotide is not shown in this figure. Panel A illustrates the denaturation step often used in thermocycling (e.g., PCR) reactions. Panel B illustrates a 3′ blocked abasic oligonucleotide species composition with a 5′ capture agent, as described herein, contacting and annealing a target nucleic acid, under annealing or hybridization conditions. Panel C illustrates a thermostable AP endonuclease (e.g., Tth Endonuclease IV in this particular embodiment) cleaving the blocked abasic oligonucleotide species composition. Panel D illustrates the unmodified forward oligonucleotide species annealing to the target nucleic acid. The steps illustrated in panels B, C and D often will occur concurrently under appropriate conditions. Panel E illustrates a thermostable DNA polymerase extending the unmodified forward oligonucleotide species; through the region of target nucleic acid annealed by the abasic “probe” oligonucleotide species, thereby displacing or aiding to displace the cleaved abasic oligonucleotide species. Panel E illustrates the completion of extension from the forward unmodified oligonucleotide. Given in each panel are non-limiting exemplary temperature ranges for each step.

FIG. 2 is a schematic representation of a method for amplifying and/or detecting a target nucleic acid using a pair of blocked abasic oligonucleotide species compositions. In the embodiment illustrated in FIG. 2, the 5′ or upstream oligonucleotide species is blocked at the 3′ end with a biotin moiety (e.g., a capture agent). The reverse or 3′ oligonucleotide species is not shown in FIG. 2, but would also be blocked with a similar or different 3′ blocking agent and/or capture agent. The oligonucleotide species compositions optionally may include a detectable feature. Panel A illustrates a denaturation step. Panel B illustrates the upstream 3′ biotin blocked abasic oligonucleotide species annealing to the target nucleic acid. Panel C illustrates a thermostable AP endonuclease (e.g., Tth Endonuclease IV in this particular embodiment) cleaving the blocked abasic oligonucleotide species composition. In the embodiment illustrated in FIG. 2, the Tm of the 3′ portion of the cleaved oligonucleotide is far enough below the Tm of the intact oligonucleotide or the 5′ portion of the cleaved oligonucleotide, that, under cleavage and extension conditions, the 3′ portion of the cleaved oligonucleotide dissociates from the target nucleic acid. Panel D illustrates the polymerase extending from the functional 5′ portion of the cleaved oligonucleotide species. Panel E illustrates the completion of extension from the cleaved oligonucleotide. Given in each panel are non-limiting exemplary temperature ranges for each step.

FIG. 3 illustrates a dual oligonucleotide species composition, which can form a stem structure, that can be used as a hybridization probe or as a blocked oligonucleotide for extension or amplification methods described herein. Shown in FIG. 3 are non-limiting exemplary melting temperatures (Tm) for various regions of the oligonucleotide species in its anneal conformation.

FIG. 4 illustrates an oligonucleotide species composition with internal stem-loop structure that can be used as a hybridization probe or as a blocked oligonucleotide for extension or amplification methods described herein. Shown in FIG. 4 are non-limiting exemplary melting temperatures (Tm) for various regions of the oligonucleotide species in its annealed conformation. The cleavage reaction illustrated in FIG. 4 can be performed by a restriction endonuclease or an AP endonuclease, depending on the cleavage site included in the oligonucleotide species composition.

FIGS. 5-9 depict the results of MALDI mass spectrometry detection of a Tth endonuclease IV cleavage of an abasic oligonucleotide species composition in an amplification reaction as described in Example 2. Specific experimental details (e.g., sequence of oligonucleotide species, type of polymerase used, reaction conditions and the like) are described in Example 2.

FIG. 10 is a schematic representation of a method for amplifying and/or detecting a target nucleic acid using an oligonucleotide species composition having a 5′ capture agent and/or detectable feature, and a thermostable restriction endonuclease cleavage substrate sequence. The method requires at least two rounds of extension before the restriction endonuclease cleavage site is formed. Panel A illustrates a denaturation step. Panel B illustrates the 5′ biotinylated oligonucleotide species annealing to the target nucleic acid. Panel C illustrates extension of the oligonucleotide species. Panel D illustrates a denaturation step, where newly synthesized extended product is denatured from the target nucleic acid. Panel E illustrates annealing of the reverse oligonucleotide. Panel F illustrates synthesis of the second extended product. Synthesis of the second extended product completes the restriction endonuclease cleavage site. Panel G illustrates cleavage by the thermostable restriction endonuclease included in the reaction. Panel I illustrates the purified cleaved fragment containing the capture agent. Given in each panel are non-limiting exemplary temperature ranges for each step.

FIG. 11. depicts the results of MALDI mass spectrometry detection of a positive reaction for cleavage of a biotinylated 5′ capture agent/detectable feature by the thermostable restriction endonuclease, Pvu II. FIGS. 12-15 depict the results of MALDI mass spectrometry detection of negative reactions for cleavage of a biotinylated 5′ capture agent/detectable feature by the thermostable restriction endonuclease, Pvu II. Specific experimental details are described in Example 3.

FIG. 16 illustrates a 3′ blocked oligonucleotide species composition pair with a restriction endonuclease cleavage site. FIG. 17 illustrates a 3′ blocked oligonucleotide species composition pair, having a 5′ tag (e.g., capture agent or detectable moiety), and a restriction site. FIG. 18 illustrates a 3′ blocked oligonucleotide species composition pair with additional intervening sequences and two different restriction endonuclease cleavage sites. FIG. 19 illustrates a 3′ blocked oligonucleotide species composition pair, having a 5′ tag, and two abasic AP endonuclease cleavage sites. FIG. 20 illustrates a 3′ blocked oligonucleotide species composition pair with additional intervening sequences and two abasic AP endonuclease cleavage sites. The embodiments illustrated in FIGS. 16-20 are useful for amplification and/or detection of target nucleic acids, and additional composition specific details are described in Example 4.

FIG. 21 is a schematic illustration of the blocked oligonucleotide species compositions being unblocked, by a thermostable AP endonuclease (e.g., Tth IV endonuclease), and generating oligonucleotides useful for extension or amplification methods. FIG. 21is further described in Example 4.

FIGS. 22 and 23 illustrate 3′ blocked oligonucleotide species duplex compositions having one or more thermostable restriction endonuclease cleavage sites useful for amplification and/or detection of target nucleic acids. FIG. 23 also illustrates an embodiment having an optional 5′ tag (e.g., capture agent and/or detectable moiety).

FIGS. 24 and 25 illustrate 3′ blocked oligonucleotide species duplex compositions having one or more thermostable AP endonuclease cleavage sites useful for amplification and/or detection of target nucleic acids. FIG. 25 also illustrates an embodiment having an optional 5′ tag (e.g., capture agent and/or detectable moiety). The embodiments illustrated in FIGS. 22-25 are useful for amplification and/or detection of target nucleic acids, and additional composition specific details are described in Example 5.

FIG. 26 is a schematic illustration of blocked oligonucleotide species compositions being unblocked and generating oligonucleotides useful for extension or amplification methods. FIG. 26 is further described in Example 5.

FIGS. 27-30A illustrate 3′ blocked J-hook oligonucleotide species compositions with endonuclease cleavage sites. FIGS. 27 and 28 contain thermostable restriction endonuclease cleavage sites. FIG. 28, also has a 5′ tag with a capture agent. FIG. 29 has a thermostable AP endonuclease cleavage site. FIG. 30A contains a thermostable nicking endonuclease cleavage site. The embodiments illustrated in FIGS. 27-29 are useful for amplification and/or detection of target nucleic acids, and additional composition specific details are described in Example 6.

FIG. 30B is a schematic illustration of J-hook oligonucleotide species compositions with thermostable nicking endonuclease cleavage sites, being unblocked and generating oligonucleotides useful for extension or amplification methods. FIG. 30B is further described in Example 6.

FIG. 31 diagrams the chemical structure of the internal spacer (e.g., Internal Spacer 18, World Wide Web Uniform Resource Locator (URL) idtdna.com) that can be used to provide additional flexibility to J-hook oligonucleotide species compositions. FIG. 32 illustrates a method for amplifying and capturing and/or detecting a target nucleic acid using a pair of 3′ blocked linear oligonucleotide species having complementary 3′ ends. Additional composition and method specific details are described in Example 6.

FIG. 33 illustrates a 3′ blocked oligonucleotide species composition with an “induced nicking function” cleavage site useful for amplification and detection of target nucleic acids. FIG. 33 is further described in Example 7.

FIGS. 34A-35C depict the results of MALDI mass spectrometry detection of 3′ blocked primers having thermostable restriction endonuclease cleavage sites. Specific experimental details are given in Example 8.

FIG. 36 illustrates a method for generating a fluorescent signal from an oligonucleotide species composition containing a thermostable restriction endonuclease and requiring at least two rounds of oligonucleotide extension.

FIG. 37 illustrates schematic examples of forward and reverse primers for detection by MALDI mass spectrometery (e.g., MassARRAY). Specific experimental details are described in Example 11. The MassARRAY detection primers used in some of the procedures described in Example 11 do not contain an internal hybridization probe. FIG. 38 illustrates a method for extending a nucleic acid past a templated abasic site using Sulfolobus DNA polymerase IV. Also illustrated in the figure is Tth endonuclease IV cleaving the abasic site generated in the double stranded DNA by the bypass of the abasic site by Sulfolobus DNA polymerase IV.

FIGS. 39-42 depict results of MALDI mass spectrometry detection of cleaved tag generated in a combined Sulfolobus DNA polymerase IV, Tth endonuclease IV and an additional DNA polymerase PCR assay. Assay conditions are described in Example 11. The additional DNA polymerases added to the reactions presented in FIGS. 39-42 are: FastStart DNA polymerase (FIG. 39); Tth DNA polymerase (FIG. 40); 9° N™m DNA polymerase (FIG. 41); and Deep vent (exo-) DNA polymerase (FIG. 42). The cleaved tag is labeled “Tag”, the passive reference spike is labeled “Spike” and the uncleaved forward primer is labeled “SRY.Dpo.Tth.f1” in the figures. Each shows the presence of the cleaved tag and indicates cleavage by the Tth Endonuclease IV enzyme.

FIG. 43 depicts calculated ratios of a SRY cleaved tag to a passive reference spike. Effects of differing PCR denaturing temperatures on this ratio are shown. FIG. 44 illustrates schematic examples of forward and reverse primers for detection using fluorescence detection. The primers illustrated in the embodiment shown in FIG. 44 and described in Example 11, include a 5′ fluorophore, an abasic site and an internal quenching moiety.

FIG. 44 depicts a schematic design for an example of a fluorescent assay utilizing a 5′ fluorescent moiety, an internal abasic site and internal quencher moiety.

### DETAILED DESCRIPTION

Methods for amplification and detection of rare or low copy number nucleic acids, including diagnostic methods such as fetal genotyping, are sometimes subject to erroneous interpretation due to false positives that can occur due to amplification artifacts. Compositions and methods described herein are useful for minimizing or eliminating amplification artifacts, and can reduce costs associated with large scale nucleic acid amplification and diagnostic testing by eliminating the need for specialized and/or costly reagents.

Compositions and methods provided herein can be used in place of, or in conjunction with other commonly used nucleic acid amplification based methods and apparatus. Compositions and methods presented herein are easily adapted for use with commonly used high throughput and automated biological workstations.

Compositions and methods provided herein are useful for amplification, capture and/or detection of target nucleic acids. Compositions and methods provided herein make use of thermostable endonucleases and blocked oligonucleotides containing cleavage sites for the endonucleases, and cleavage by the endonuclease allows amplification and detection of nucleic acids. Compositions and methods described herein do not require partitioning reactants or using polymerase inhibitors, or specialized “hot start” procedures. Compositions provided herein also can include capture agents and detectable features to allow for a wide range of applicability for laboratory and clinical diagnostic procedures.

In addition to eliminating the need for partitioned or inhibited reaction components, or other “hot start” techniques, compositions and methods provided herein also impart the following representative advantages: (i) single or closed tube reactions (e.g., all components work in substantially similar conditions, no need to interrupt a thermocycling profile to add additional components, or to move all or a part of the reaction to another reaction vessel), (ii) flexibility of oligonucleotide species design due to the number of thermostable endonucleases available (e.g., AP endonucleases, restriction endonucleases and nicking endonucleases), (iii) readily adaptable to allow use of a wide variety of capture and/or detection methods (e.g., a wide variety of capture agents and detectable features can be incorporated into the oligonucleotide compositions), and (iv) ease of reaction set up (e.g., in many instances, annealing, cleavage and extension conditions are substantially similar).

Compositions and methods described herein can be used without reaction partitioning, polymerase inhibitors or other hot start approaches. In some embodiments, however, hot start procedures (e.g., use of an antibody or chemical to inactivate DNA polymerase until a certain temperature is reached) can be used in conjunction with the compositions and methods described herein for added reaction specificity.

In addition to advantages listed above, compositions and methods provided herein can be used to routinely screen for thermostable endonucleases that can be induced to “nick” DNA. Restriction endonucleases typically cleave both strands of DNA in or near the restriction endonuclease recognition site. Nicking endonucleases typically cleave only a single strand of DNA in, or near the nicking endonuclease recognition site. Compositions and methods using non-cleavable nucleotide analogs are described herein that allow for routine screening of thermostable restriction endonucleases for the ability to cleave only a single strand of DNA in a double-stranded recognition site.

Sample or Target Nucleic Acids and Nucleic Acid Compositions

A nucleic acid composition can comprise any type of nucleic acid or mixture of different types of nucleic acids. A nucleic acid composition can be from a sample. Sample nucleic acid may be derived from one or more samples or sources. As used herein, “nucleic acid” refers to polynucleotides such as deoxyribonucleic acid (DNA) and ribonucleic acid (RNA). The term should also be understood to include, as equivalents, derivatives, variants and analogs of RNA or DNA made from nucleotide analogs, single (sense or antisense) and double-stranded polynucleotides. It is understood that the term “nucleic acid” does not refer to or infer a specific length of the polynucleotide chain, thus nucleotides, polynucleotides, and oligonucleotides are also included in the definition. Deoxyribonucleotides include deoxyadenosine, deoxycytidine, deoxyguanosine and deoxythymidine. For RNA, the uracil base is uridine. A source or sample containing sample nucleic acid(s) may contain one or a plurality of sample nucleic acids. A plurality of sample nucleic acids as described herein refers to at least 2 sample nucleic acids and includes nucleic acid sequences that may be identical or different. That is, the sample nucleic acids may all be representative of the same nucleic acid sequence, or may be representative of two or more different nucleic acid sequences (e.g., from 1, 2, 3, 4, 5, 6, 7, 8, 9, 10, 11, 12, 13, 14, 15, 16, 17, 18, 19, 20, 50, 100, 1000 or more sequences).

A sample may be collected from an organism, mineral or geological site (e.g., soil, rock, mineral deposit, combat theater), forensic site (e.g., crime scene, contraband or suspected contraband), or a paleontological or archeological site (e.g., fossil, or bone) for example. A sample may be a “biological sample,” which refers to any material obtained from a living source or formerly-living source, for example, an animal such as a human or other mammal, a plant, a bacterium, a fungus, a protist or a virus. The biological sample can be in any form, including without limitation a solid material such as a tissue, cells, a cell pellet, a cell extract, or a biopsy, or a biological fluid such as urine, blood, saliva, amniotic fluid, exudate from a region of infection or inflammation, or a mouth wash containing buccal cells, urine, cerebral spinal fluid and synovial fluid and organs.

The biological sample can be maternal blood, including maternal plasma or serum. In some circumstances, the biological sample is acellular. In other circumstances, the biological sample does contain cellular elements or cellular remnants in maternal blood. Other biological samples include amniotic fluid, chorionic villus sample, biopsy material from a pre-implantation embryo, maternal urine, maternal saliva, a celocentesis sample, fetal nucleated cells or fetal cellular remnants, or the sample obtained from washings of the female reproductive tract. In some embodiments, a biological sample may be blood, and sometimes plasma.

As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation in such embodiments. A fluid or tissue sample from which template nucleic acid is extracted may be acellular. In some embodiments, a fluid or tissue sample may contain cellular elements or cellular remnants.

For prenatal applications of technology described herein, fluid or tissue sample may be collected from a female at a gestational age suitable for testing, or from a female who is being tested for possible pregnancy. Suitable gestational age may vary depending on the chromosome abnormality tested. In certain embodiments, a pregnant female subject sometimes is in the first trimester of pregnancy, at times in the second trimester of pregnancy, or sometimes in the third trimester of pregnancy. In certain embodiments, a fluid or tissue is collected from a pregnant woman at 1-4, 4-8, 8-12, 12-16, 16-20, 20-24, 24-28, 28-32, 32-36, 36-40, or 40-44 weeks of fetal gestation, and sometimes between 5-28 weeks of fetal gestation.

Template nucleic acid can be extracellular nucleic acid in certain embodiments. The term “extracellular template nucleic acid” as used herein refers to nucleic acid isolated from a source having substantially no cells (e.g., no detectable cells; may contain cellular elements or cellular remnants). Examples of acellular sources for extracellular nucleic acid are blood plasma, blood serum and urine. Without being limited by theory, extracellular nucleic acid may be a product of cell apoptosis and cell breakdown, which provides basis for extracellular nucleic acid often having a series of lengths across a large spectrum (e.g., a “ladder”).

Extracellular template nucleic acid can include different nucleic acid species. For example, blood serum or plasma from a person having cancer can include nucleic acid from cancer cells and nucleic acid from non-cancer cells. In another example, blood serum or plasma from a pregnant female can include maternal nucleic acid and fetal nucleic acid. In some instances, fetal nucleic acid sometimes is about 5% to about 40% of the overall template nucleic acid (e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39% of the template nucleic acid is fetal nucleic acid). In some embodiments, the majority of fetal nucleic acid in template nucleic acid is of a length of about 500 base pairs or less (e.g., about 80, 85, 90, 91, 92, 93, 94, 95, 96, 97, 98, 99 or 100% of fetal nucleic acid is of a length of about 500 base pairs or less).

Low copy number or rare target nucleic acid sometimes is detected. In certain embodiments, a rare mutation (for example, a cancer mutation) is detected in a relatively large background of non-cancer, wild-type nucleic acid, and utilized to detect the presence or absence of cancer. Likewise, a fetal-specific nucleic acid (for example, a polymorphism present in fetal nucleic acid but not in maternal nucleic acid) is detected in a relatively large background of maternal nucleic acid, and utilized to detect the presence or absence of a fetal disorder, characteristic or abnormality. Methods for detecting low copy number or rare nucleic acid include taking advantage of oligonucleotides that selectively block the amplification or detection of wild-type or background nucleic acid.

The amount of fetal nucleic acid (e.g., concentration) in template nucleic acid sometimes is determined. In certain embodiments, the amount of fetal nucleic acid is determined according to markers specific to a male fetus (e.g., Y-chromosome STR markers (e.g., DYS 19, DYS 385, DYS 392 markers); RhD marker in RhD-negative females), or according to one or more markers specific to fetal nucleic acid and not maternal nucleic acid (e.g., fetal RNA markers in maternal blood plasma; Lo, 2005, Journal of Histochemistry and Cytochemistry 53 (3): 293-296). The amount of fetal nucleic acid in extracellular template nucleic acid can be quantified and utilized for the identification of the presence or absence of a chromosome abnormality in certain embodiments.

In some embodiments, extracellular nucleic acid is enriched or relatively enriched for fetal nucleic acid. Methods for enriching a sample for a particular species of nucleic acid are described in PCT Patent Application Number PCT/US07/69991, filed May 30, 2007, PCT Patent Application Number PCT/US2007/071232, filed Jun. 15, 2007, PCT Patent Publication Numbers WO 2009/032779 and WO 2009/032781, both filed Aug. 28, 2008, PCT Patent Publication Number WO 2008/118988, filed Mar. 26, 2008, and PCT Patent Application Number PCT/EP05/012707, filed Nov. 28, 2005. In certain embodiments, maternal nucleic acid is selectively removed (partially, substantially, almost completely or completely) from the sample. In other certain embodiments, fetal nucleic acid is selectively amplified (partially, substantially, almost completely or completely) from the sample.

A sample also may be isolated at a different time point as compared to another sample, where each of the samples are from the same or a different source. A sample nucleic acid may be from a nucleic acid library, such as a cDNA or RNA library, for example. A sample nucleic acid may be a result of nucleic acid purification or isolation and/or amplification of nucleic acid molecules from the sample. Sample nucleic acid provided for sequence analysis processes described herein may contain nucleic acid from one sample or from two or more samples (e.g., from 1, 2, 3, 4, 5, 6, 7, 8, 9, 10, 11, 12, 13, 14, 15, 16, 17, 18, 19 or 20 samples).

Sample nucleic acid may comprise or consist essentially of any type of nucleic acid suitable for use with processes of the technology, such as sample nucleic acid that can hybridize to solid phase nucleic acid (described hereafter), for example. A sample nucleic in certain embodiments can comprise or consist essentially of DNA (e.g., complementary DNA (cDNA), genomic DNA (gDNA) and the like), RNA (e.g., message RNA (mRNA), short inhibitory RNA (siRNA), ribosomal RNA (rRNA), tRNA and the like), and/or DNA or RNA analogs (e.g., containing base analogs, sugar analogs and/or a non-native backbone and the like). A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, a cell, a cell nucleus or cytoplasm of a cell in certain embodiments. A sample nucleic acid in some embodiments is from a single chromosome (e.g., a nucleic acid sample may be from one chromosome of a sample obtained from a diploid organism).

Sample nucleic acid may be provided for conducting methods described herein without processing of the sample(s) containing the nucleic acid in certain embodiments. In some embodiments, sample nucleic acid is provided for conducting methods described herein after processing of the sample(s) containing the nucleic acid. For example, a sample nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered “by the hand of man” from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Sample nucleic acid also may be processed by subjecting nucleic acid to a method that generates nucleic acid fragments, in certain embodiments, before providing sample nucleic acid for a process described herein. In some embodiments, sample nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method known in the art, and the average, mean or nominal length of nucleic acid fragments can be controlled by selecting an appropriate fragment-generating procedure by the person of ordinary skill. In certain embodiments, sample nucleic acid of a relatively shorter length can be utilized to analyze sequences that contain little sequence variation and/or contain relatively large amounts of known nucleotide sequence information. In some embodiments, sample nucleic acid of a relatively longer length can be utilized to analyze sequences that contain greater sequence variation and/or contain relatively small amounts of unknown nucleotide sequence information.

Sample nucleic acid fragments often contain overlapping nucleotide sequences, and such overlapping sequences can facilitate construction of a nucleotide sequence of the previously non-fragmented sample nucleic acid, or a portion thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample. Sample nucleic acid may be partially fragmented (e.g., from an incomplete or terminated specific cleavage reaction) or fully fragmented in certain embodiments.

Sample nucleic acid can be fragmented by various methods known to the person of ordinary skill, which include without limitation, physical, chemical and enzymic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected by the person of ordinary skill to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Sample nucleic acid may be specifically cleaved by contacting the nucleic acid with one or more specific cleavage agents. The term “specific cleavage agent” as used herein refers to an agent, sometimes a chemical or an enzyme, that can cleave a nucleic acid at one or more specific sites. Specific cleavage agents often will cleave specifically according to a particular nucleotide sequence at a particular site.

Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I, Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I.); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

As used herein, the term “complementary cleavage reactions” refers to cleavage reactions that are carried out on the same sample nucleic acid using different cleavage reagents or by altering the cleavage specificity of the same cleavage reagent such that alternate cleavage patterns of the same target or reference nucleic acid or protein are generated. In certain embodiments, sample nucleic acid may be treated with one or more specific cleavage agents (e.g., 1, 2, 3, 4, 5, 6, 7, 8, 9, 10 or more specific cleavage agents) in one or more reaction vessels (e.g., sample nucleic acid is treated with each specific cleavage agent in a separate vessel).

Sample nucleic acid also may be exposed to a process that modifies certain nucleotides in the nucleic acid before providing sample nucleic acid for a method described herein. A process that selectively modifies nucleic acid based upon the methylation state of nucleotides therein can be applied to sample nucleic acid. The term “methylation state” as used herein refers to whether a particular nucleotide in a polynucleotide sequence is methylated or not methylated. Methods for modifying a target nucleic acid molecule in a manner that reflects the methylation pattern of the target nucleic acid molecule are known in the art, as exemplified in U.S. Pat. No. 5,786,146 and U.S. patent publications 20030180779 and 20030082600. For example, non-methylated cytosine nucleotides in a nucleic acid can be converted to uracil by bisulfite treatment, which does not modify methylated cytosine. Non-limiting examples of agents that can modify a nucleotide sequence of a nucleic acid include methylmethane sulfonate, ethylmethane sulfonate, diethylsulfate, nitrosoguanidine (N-methyl-N′-nitro-N-nitrosoguanidine), nitrous acid, di-(2-chloroethyl)sulfide, di-(2-chloroethyl)methylamine, 2-aminopurine, t-bromouracil, hydroxylamine, sodium bisulfite, hydrazine, formic acid, sodium nitrite, and 5-methylcytosine DNA glycosylase. In addition, conditions such as high temperature, ultraviolet radiation, x-radiation, can induce changes in the sequence of a nucleic acid molecule.

Sample nucleic acid may be provided in any form useful for conducting a sequence analysis or manufacture process described herein, such as solid or liquid form, for example. In certain embodiments, sample nucleic acid may be provided in a liquid form optionally comprising one or more other components, including without limitation one or more buffers or salts selected by the person of ordinary skill. The terms “sample”, “sample nucleic acid”, “target” and “target nucleic acid” can be used interchangeably through the document.

Endonucleases

Endonucleases are enzymes that cleave the phosphodiester bond within a polynucleotide chain, in contrast to exonucleases, which cleave phosphodiester bonds at the end of a polynucleotide chain. Non-limiting examples of endonucleases are restriction endonucleases, Apurinic/apyrimidinc (AP) endonucleases, and nicking endonucleases. Thermostable or heat tolerant endonucleases have been identified and are commercially available from a number of sources. Thermostable and heat tolerant endonucleases are of particular interest for use in the compositions and methods provided herein. Thermostable restriction endonuclease, AP endonucleases and nicking endonucleases can be used in extension and amplification reaction to increase reaction specificity by eliminating amplification artifacts through the use of site-specific endonuclease cleavage, under amplification condition. In some embodiments, the thermostable endonucleases may serve to “unblock” blocked extension oligonucleotides, which then allows extension by a thermostable DNA polymerase, thereby generating a specific product by eliminating spurious priming artifacts, under amplification conditions. In some embodiments, the thermostable endonucleases can serve to eliminate “primer dimers”, where the sequence of the oligonucleotide species composition includes a restriction endonuclease cleavage site that is generated or regenerated upon formation of “primer-dimer” type artifacts. In some embodiments, the thermostable endonucleases may be included in extension or amplification based protocols, to liberate fragments containing capture agents or detectable features, or to distinguish between allelic variants. For example, allelic variants can be distinguished using compositions and methods described herein, in conjunction with the thermostable T7 endonuclease I, which will cleave unpaired nucleotides in a region of double stranded DNA. This is particularly useful for genotypic screening, as SNP's typically can distinguish between allelic variants that differ by only 1 nucleotide. Using oligonucleotides based on SNP sequences for a particular locus would allow design of extension oligonucleotides that can be used to distinguish between alleles during the amplification process by cleaving mismatched oligonucleotide sequences, and allowing detection of the presence or absence of a particular allele.

As used herein, the terms “heat tolerant” or “heat tolerance” refer to an enzyme that can function at moderate temperatures (e.g., 50 C to 60 C), but will lose activity under non-isothermal amplification conditions, which include one or more denaturation steps (e.g., 90 C to 95 C). Heat tolerant endonucleases often require prolonged incubation temperatures above 65 C to 70 C for inactivation. As used herein, the term “thermostable” refers to an enzyme that has enzymatic activity after exposure to elevated temperature (e.g., greater than 65 C, for example) or after repeated exposure at elevated temperatures, such as in amplification conditions, for example. Thermostability with respect to endonucleases can be expressed in terms of a heat tolerant half-life of an enzyme. The term “heat tolerant half-life” refers to the length of time an enzyme may be incubated at an elevated temperature and recover at least 50% of its enzymatic activity. That is, a thermostable endonuclease sometimes can lose less than about 50% of its activity, under amplification conditions. The term “heat tolerant half-life” also refers to the number of times an enzyme can be cycled, under amplification conditions, before losing greater than 50% of its activity. The heat tolerant half-life of endonucleases often differs with the temperature of incubation, where typically higher temperatures (e.g., 80 C or 90 C) result in a shorter half-life (e.g., fewer number of cycles) than incubation at more moderate temperatures (e.g., 70 C). Examples of thermostable endonucleases are described herein, and multiple endonucleases can be readily screened to determine whether they are thermostable (e.g., a test endonuclease can be exposed briefly to an elevated temperature one or more times, and endonuclease activity can be assessed thereafter).

Restriction endonucleases (e.g., restriction enzymes) typically cleave double stranded DNA at specific sites, typically associated with a specific, or substantially specific recognition sequence. Some restriction enzymes can cleave single stranded DNA (e.g., nicking endonucleases). Restriction enzymes, found in bacteria and archaea, are thought to have evolved to provide a defense mechanism against invading viruses. Inside a bacterial host, the restriction enzymes selectively cleave foreign DNA in a process called restriction; host DNA is methylated by a modification enzyme (a methylase) to protect it from the restriction enzyme's activity. The term “recognition site” as used herein, refers to the specific nucleotide sequence recognized and bound by the endonuclease. The term “cleavage site” as used herein, refers to the site where the single or double stranded cleavage is made by the endonuclease. In some embodiments, the recognition site will contain the cleavage site. In certain embodiments the cleavage site will be adjacent to or near the recognition site. The terms “adjacent” and “near” are defined below. Depending on the restriction enzyme, the specific DNA sequence, which is recognized and then cleaved, typically varies from 4 and 8 bases in length, but some recognition sequences are longer. Cleavage by a restriction enzyme produces either cohesive (having either a 5′ or 3′ single˜stranded protrusion) or blunt ended (no single stranded protrusion) fragments. Cohesive or protruding ends are commonly referred to as “Sticky ends” and ends with no single stranded protrusion are commonly referred to as “blunt ends”. Sticky ended fragments posses' 3′ or 5′ overhangs which can “stick” together and are useful if ends are to be ligated for cloning or other molecular biology methods. Blunt ended fragments do not have overhangs, but their ends can still be useful for various molecular biology methods, including DNA polymerase extension (e.g., priming hydroxyl for extension or amplification reactions, for example). Restriction enzymes are divided into three categories, Type I, Type II, and Type III, according to their mechanism of action.

Type I enzymes are complex, multi-subunit, combination restriction and modification enzymes that cut DNA at random far from their recognition sequences. Originally thought to be rare, these enzymes are now known to be common from the analysis of sequenced genomes. Type I enzymes do not produce discrete restriction fragments or distinct gel banding patterns. Type III enzymes are also large combination restriction and modification enzymes. They cleave outside of their recognition sequences and require two such sequences in opposite orientations within the same DNA molecule to accomplish cleavage, and they rarely give complete digests.

Type II enzymes are of the most interest due to the large number available, the variety of recognition sites and the finding that many type II enzymes are heat tolerant or thermostable. Type II enzymes cut DNA at defined positions close to or within their recognition sequences. They produce discrete restriction fragments and distinct gel banding patterns, and they are the only class used in the laboratory for DNA analysis and gene cloning. Rather then forming a single family of related proteins, type II enzymes are a collection of unrelated proteins of many different sorts. Type II enzymes frequently differ so utterly in amino acid sequence from one another, and indeed from every other known protein, that they likely arose independently in the course of evolution rather than diverging from common ancestors.

The most common type II enzymes are those like HhaI, HindIII and NotI that cleave DNA within their recognition sequences. Enzymes of this kind are the principal ones available commercially. Most recognize DNA sequences that are symmetric because they bind to DNA as homodimers, but a few, (e.g., BbvCl: CCTCAGC) recognize asymmetric DNA sequences because they bind as heterodimers. Some enzymes recognize continuous sequences (e.g., EcoRI: GAATTC) in which the two half-sites of the recognition sequence are adjacent, while others recognize discontinuous sequences (e.g., BglI: GCCNNNNNGGC) in which the half-sites are separated. Cleavage leaves a 3′˜hydroxyl on one side of each cut and a 5′˜phosphate on the other. They require only magnesium for activity and the corresponding modification enzymes require only S˜adenosylmethionine. They tend to be small, with subunits in the 200˜350 amino acid range.

The next most common type II enzymes, sometimes referred to as “type IIs” are those like Fokl and AlwI that cleave outside of their recognition sequence to one side. These enzymes are intermediate in size, 400˜650 amino acids in length, and they recognize sequences that are continuous and asymmetric. They comprise two distinct domains, one for DNA binding, and the other for DNA cleavage. They are thought to bind to DNA as monomers and to cleave DNA cooperatively, through dimerization of the cleavage domains of adjacent enzyme molecules. For this reason, some type Ils enzymes are much more active on DNA molecules that contain multiple recognition sites.

The third major kind of type II enzyme, more properly referred to as “type IV” are large, combination restriction and modification enzymes, 850-1250 amino acids in length, in which the two enzymatic activities reside in the same protein chain. These enzymes cleave outside of their recognition sequences; those that recognize continuous sequences (e.g., AcuI: CTGAAG) cleave on just one side; those that recognize discontinuous sequences (e.g., BcgI: CGANNNNNNTGC) cleave on both sides releasing a small fragment containing the recognition sequence. The amino acid sequences of these enzymes are varied but their organization are consistent. They comprise an N-terminal DNA cleavage domain joined to a DNA modification domain and one or two DNA sequence specificity domains forming the C-terminus, or present as a separate subunit. When these enzymes bind to their substrates, they switch into either restriction mode to cleave the DNA, or modification mode to methylate it.

Non-limiting examples of useful heat tolerant and/or thermostable restriction endonucleases are; Ack 1, Apa LI, Ape KI, Bam HI, Bam HI-HF, Bcl I, Bgl II, Blp I, Bsa Al, Bsa XI, Bsi HKAI, Bso BI, Bsr FI, Bst BI, Bst EII, Bst NI, Bst UI, Bst Z17I, Bts CI, Cvi QI, Hpa I, Kpn I, Mwo I, Nci I, Pae R7I, Pho I, Ppu MI, Pvu II, Sfi I, Sfo I, Sml I, Tti I, Tsp 5091, Tsp MI, Tsp RI, and Zra I. Apurinic/apyrimidinc (AP) endonucleases also can cleave DNA at specific sites, typically associated with an abasic site. As used herein, the terms “abasic nucleic acid” or “abasic site” or “abasic oligonucleotide” refers to a nucleic acid composition that has one or more nucleosides (e.g., nucleobase, adenine, guanine, cytosine, or thymine, for example) removed from the nucleic acid chain, leaving the backbone intact. Abasic sites typically are repaired in vivo by the DNA base excision repair pathway (BER) of which AP endonucleases are a part. The main role of AP endonucleases in the repair of damaged or mismatched nucleotides in DNA is to create a nick in the phosphodiester backbone of the AP site created when DNA glycosylase removes the damaged base. There are four types of AP endonucleases which have been classified according to their sites of incision. Class I and class II AP endonucleases incise DNA at the phosphate groups 3′ and 5′ to the baseless site leaving 3′-OH and 5′-phosphate termini. Class III and class IV AP endonucleases also cleave DNA at the phosphate groups 3′ and 5′ to the baseless site, but generate a 3′-phosphate and a 5′-OH. The AP endonucleases suitable for use with compositions and embodiments described herein generate 3′ hydroxyls (e.g., —OH) that can be used for extension in extension or amplification reactions, under extension and/or amplification conditions (e.g., class I and class II AP endonucleases). Non-limiting examples of thermostable AP endonucleases are Tth endonuclease IV, and the AP endonucleases from Thermotogoa maritime, Thermoplasm volacanium and lactobacillus plantarum. AP endonucleases often cleave only one strand of a double-stranded target sequence.

In addition to AP endonucleases, certain sequence specific endonucleases cleave only one strand of a double stranded target sequence. These endonucleases are sometimes referred to as nicking endonucleases. Nicking endonucleases are commercially available (New England BioLabs, World Wide Web URL neb.com). Non-limiting examples of nicking enzymes useful for compositions and methods described herein are Nb. BsmI, and Nb.BrsDI. Additional non-limiting examples of useful thermostable endonucleases are E. coli endonuclease V, and T7 endonuclease I. Endonuclease V is a repair enzyme that cleaves DNA containing deoxyinosine (paired or unpaired on double stranded and will also cleave single stranded to a lesser extent), DNA containing abasic sites or urea, base mismatches, insertion/deletion mismatches, hairpin or unpaired loops, flaps and pseudo-Y structures. T7 endonuclease I, recognizes and cleaves non-perfectly matched DNA, cruciform DNA structures, Holliday structures or junctions, heteroduplex DNA and more slowly, nicked double-stranded DNA. The cleavage site is at first, second or third phosphodiester bond that is 5′ to the mismatch. T7 endonuclease 1 can also cleave linear single stranded DNA (especially if the single stranded DNA folds back on itself), small loops (4-15 bases) misaligned primers, and supercoiled circular DNA (slowly due to the resistance to nicking). Linear duplex DNA is not cleaved by T7 endonuclease I.

As described herein, certain endonucleases that cleave both strands of a double-stranded target nucleic acid can be induced to cleave only one strand of the target by incorporation of one or more cleavage-resistant nucleotides in one strand of the target. In the latter embodiments, the endonuclease that normally cleaves both strands will not cleave the strand that includes such nucleotide analogs, and will cleave the strand that does not include the nucleotide analogs. Non-limiting examples of nucleotide analogs that cannot be cleaved include peptide nucleic acid (PNA), phosphosphorotioates and locked nucleic acids (e.g., the ribose moiety is modified with a bridge connecting the 2′ and 4′ carbons).

Amplification

In some embodiments, it may be desirable to amplify the target sequence using any of several nucleic acid amplification procedures (described in greater detail below). Nucleic acid amplification may be particularly beneficial when target sequences exist at low copy number, or the target sequences are non-host sequences and represent a small portion of the total nucleic acid in the sample (e.g., fetal nucleic acid in a maternal nucleic acid background). In some embodiments, amplification of target sequences may aid in detection of gene dosage imbalances, as might be seen in genetic disorders involving chromosomal aneuploidy, for example.

Nucleic acid amplification often involves enzymatic synthesis of nucleic acid amplicons (copies), which contain a sequence complementary to a nucleotide sequence species being amplified. An amplification product (amplicon) of a particular nucleotide sequence species (e.g., target sequence) is referred to herein as an “amplified nucleic acid species.” Amplifying target sequences and detecting the amplicon synthesized, can improve the sensitivity of an assay, since fewer target sequences are needed at the beginning of the assay, and can improve detection of target sequences.

The terms “amplify”, “amplification”, “amplification reaction”, or “amplifying” refers to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments, a one-time, single oligonucleotide extension step can be used to generate a double stranded nucleic acid feature (e.g., synthesize the complement of a restriction endonuclease cleavage site contained in a single stranded oligonucleotide species, thereby creating a restriction site).

In some embodiments, a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions, and also may reduce amplification biases due to nucleotide sequence or species abundance of the target. In some embodiments, a one-time primer extension may be used may be performed as a prelude to linear or exponential amplification. In some embodiments, amplification of the target nucleic acid may not be required, due to the use of ultra sensitive detections methods (e.g., single nucleotide sequencing, sequencing by synthesis and the like).

Where amplification may be desired, any suitable amplification technique can be utilized. Non-limiting examples of methods for amplification of polynucleotides include, polymerase chain reaction (PCR); ligation amplification (or ligase chain reaction (LCR)); amplification methods based on the use of Q-beta replicase or template-dependent polymerase (see US Patent Publication Number US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Biased Allele-Specific (BAS) Amplification, which is described in PCT Patent Publication No. WO 20071147063A2 filed Jun. 14, 2007 and is hereby incorporated by reference, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, Universal Size-Specific PCR (USS-PCR), which is described in PCT Patent Application No. WO 2009/032781 filed Aug. 28, 2008 and is hereby incorporated by reference, combinations thereof, and the like. Reagents and hardware for conducting PCR are commercially available.

In some embodiments, amplification target nucleic acid may be accomplished by any suitable method available to one of skill in the art or selected from the listing above (e.g., ligase chain reaction (LCR), transcription-mediated amplification, and self-sustained sequence replication or nucleic acid sequence-based amplification (NASBA)). More recently developed branched-DNA technology also may be used to amplify the signal of target nucleic acids. For a review of branched-DNA (bDNA) signal amplification for direct quantification of nucleic acid sequences in clinical samples, see Nolte, Adv. Clin. Chem. 33:201-235, 1998.

Amplification also can be accomplished using digital PCR, in certain embodiments (e.g., Kalinina and colleagues (Kalinina et al., “Nanoliter scale PCR with TaqMan detection.” Nucleic Acids Research. 25; 1999-2004, (1997); Vogelstein and Kinzler (Digital PCR. Proc Natl Acad Sci U S A. 96; 9236-41, (1999); PCT Patent Publication No. WO05023091A2 (incorporated herein in its entirety); US Patent Publication No. 20070202525 (incorporated herein in its entirety)). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation).

In some embodiments, where RNA nucleic acid species may be used for detection of fetal sequences, a DNA copy (cDNA) of the RNA transcripts of interest can be synthesized prior to the amplification step. The cDNA copy can be synthesized by reverse transcription, which may be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, Minn., 1993; Egger et al., J. Clin. Microbiol. 33:1442-1447, 1995; and U.S. Pat. No. 5,075,212.

Use of a primer extension reaction also can be applied in methods described herein. A primer extension reaction operates, for example, by discriminating nucleic acid sequences, SNP alleles for example, at a single nucleotide mismatch (e.g., a mismatch between paralogous sequences, or SNP alleles). The terms “paralogous sequence” or “paralogous sequences” refer to sequences that have a common evolutionary origin but which may be duplicated over time in the genome of interest. Paralogous sequences may conserve gene structure (e.g., number and relative position of introns and exons and preferably transcript length), as well as sequence. Therefore, the methods described herein can be used to detect sequence mismatches in SNP-alleles or in evolutionarily conserved regions that differ by one or more point mutations, insertions or deletions (both will hereinafter be referred to as “mismatch site” or “sequence mismatch”).

The mismatch may be detected by the incorporation of one or more deoxynucleotides and/or dideoxynucleotides to a primer extension primer or oligonucleotide species, which hybridizes to a region adjacent to the SNP site (e.g., mismatch site). The extension oligonucleotide generally is extended with a polymerase. In some embodiments, a detectable tag, detectable moiety or detectable moiety is incorporated into the extension oligonucleotide or into the nucleotides added on to the extension oligonucleotide (e.g., biotin or streptavidin). The extended oligonucleotide can be detected by any known suitable detection process (e.g., mass spectrometry; sequencing processes). In some embodiments, the mismatch site is extended only by one or two complementary deoxynucleotides or dideoxynucleotides that are tagged by a specific label or generate a primer extension product with a specific mass, and the mismatch can be discriminated and quantified.

For embodiments using primer extension methods to amplify a target sequence, the extension of the oligonucleotide species is not limited to a single round of extension, and is therefore distinguished from “one-time primer extension” described above. Non-limiting examples of primer extension or oligonucleotide extension methods suitable for use with embodiments described herein are described in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039, for example.

A generalized description of an amplification process is presented herein. Oligonucleotide species compositions described herein and target nucleic acid are contacted, and complementary sequences anneal to one another, for example. Oligonucleotide can anneal to a nucleic acid, at or near (e.g., adjacent to, abutting, and the like) a target sequence of interest. A reaction mixture, containing all components necessary for full enzymatic functionality, is added to the oligonucleotide species—target nucleic acid hybrid, and amplification can occur under suitable conditions. Components of an amplification reaction may include, but are not limited to, e.g., oligonucleotide species compositions (e.g., individual oligonucleotides, oligonucleotide pairs, oligonucleotide sets and the like) a polynucleotide template (e.g., nucleic acid containing a target sequence), polymerase, nucleotides, dNTPs, an appropriate endonuclease and the like. Extension conditions are sometimes a subset of, or substantially similar to amplification conditions.

In some embodiments, non-naturally occurring nucleotides or nucleotide analogs, such as analogs containing a detectable moiety or feature (e.g., fluorescent or colorimetric label) may be used, for example. In some embodiments, non-naturally occurring nucleotides or nucleotide analogs, such as analogs containing a detectable moiety or feature (e.g., fluorescent or colorimetric label) may be used, for example. In some embodiments, primer oligonucleotides are modified, for example, to facilitate “hot start” PCR. Examples of modified primer oligonucleotides are disclosed in US Patent Application No 11/583,605, which published as US 20070219361A1. Nucleotides may also be modified, for example, according to the methods described in U.S. Pat. No. 6,762,298.

Polymerases can be selected by a person of ordinary skill and include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR, see for example, U.S. Pat. Nos. 5,677,152 and 5,772,58); Arrow™ Taq DNA Polymerase (high sensitivity and long template amplification), JumpStart Taq™ (combination of AccuTaq LA DNA Polymerase and a Taq-directed antibody), 9° N™m DNA polymerase (e.g., engineered polymerase with decreased 3′-5′ proofreading exonuclease activity), Deep Vent_{R}™ (exo-) DNA polymerase (e.g., engineered polymerase with decreased 3′-5′ proofreading exonuclease activity), Tth DNA polymerase (e.g., possesses a 5′ to 3′ exonuclease activity), antibody-mediated polymerases such as those described in U.S. Pat. Nos. 5,338,671 and 5,587,287) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

The terms “near” or “adjacent to” when referring to a nucleotide target sequence refers to a distance or region between the end of the primer and the nucleotide or nucleotides of interest. As used herein adjacent is in the range of about 5 nucleotides to about 500 nucleotides (e.g., about 5 nucleotides away from nucleotide of interest, about 10, about 20, about 30, about 40, about 50, about 60, about 70, about 80, about 90, about 100, about 150, about 200, about 250, about 300, abut 350, about 400, about 450 or about 500 nucleotides from a nucleotide of interest).

Each amplified nucleic acid species independently can be about 10 to about 1000 base pairs in length in some embodiments. In certain embodiments, an amplified nucleic acid species is about 20 to about 250 base pairs in length, sometimes is about 50 to about 150 base pairs in length and sometimes is about 100 base pairs in length. Thus, in some embodiments, the length of each of the amplified nucleic acid species products independently is about 10, 15, 20, 25, 30, 35, 40, 45, 50, 55, 60, 65, 70, 75, 80, 82, 84, 86, 88, 90, 92, 94, 96, 98, 100, 102, 104, 106, 108, 110, 112, 114, 116, 118, 120, 125, 130, 135, 140, 145, 150, 175, 200, 250, 300, 350, 400, 450, 500, 550, 600, 650, 700, 750, 800, 850, 900, 950 or 1000 base pairs (bp) in length.

An amplification product may include naturally occurring nucleotides, non-naturally occurring nucleotides, nucleotide analogs and the like and combinations of the foregoing. An amplification product often has a nucleotide sequence that is identical to or substantially identical to a target sequence or complement thereof. A “substantially identical” nucleotide sequence in an amplification product will generally have a high degree of sequence identity to the nucleotide sequence species being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Additional PCR protocols are described in the example section. Multiple cycles frequently are performed using a commercially available thermal cycler. Suitable isothermal amplification processes known and selected by the person of ordinary skill in the art also may be applied, in certain embodiments.

In some embodiments, multiplex amplification processes may be used to amplify target sequences, such that multiple amplicons are simultaneously amplified in a single, homogenous reaction. As used herein “multiplex amplification” refers to a variant of PCR where simultaneous amplification of many target sequences in one reaction vessel may be accomplished by using more than one pair of primers (e.g., more than one primer set). Multiplex amplification may be useful for analysis of deletions, mutations, and polymorphisms, or quantitative assays, in some embodiments. In certain embodiments multiplex amplification may be used for detecting paralog sequence imbalance, genotyping applications where simultaneous analysis of multiple markers is required, detection of pathogens or genetically modified organisms, or for microsatellite analyses. In some embodiments multiplex amplification may be combined with another amplification (e.g., PCR) method (e.g., nested PCR or hot start PCR, for example) to increase amplification specificity and reproducibility. In some embodiments, multiplex amplification processes may be used to amplify the Y-chromosome loci described herein.

In certain embodiments, nucleic acid amplification can generate additional nucleic acid species of different or substantially similar nucleic acid sequence. In certain embodiments described herein, contaminating or additional nucleic acid species, which may contain sequences substantially complementary to, or may be substantially identical to, the target sequence, can be useful for sequence quantification, with the proviso that the level of contaminating or additional sequences remains constant and therefore can be a reliable marker whose level can be substantially reproduced. Additional considerations that may affect sequence amplification reproducibility are; PCR conditions (number of cycles, volume of reactions, melting temperature difference between primers pairs, and the like), concentration of target nucleic acid in sample (e.g. fetal nucleic acid in maternal nucleic acid background, viral nucleic acid in host background), the number of chromosomes on which the nucleotide species of interest resides (e.g., paralogous sequences or SNP-alleles), variations in quality of prepared sample, and the like. The terms “substantially reproduced” or “substantially reproducible” as used herein refer to a result (e.g., quantifiable amount of nucleic acid) that under substantially similar conditions would occur in substantially the same way about 75% of the time or greater, about 80%, about 85%, about 90%, about 95%, or about 99% of the time or greater.

In some embodiments, amplification may be performed on a solid support. In some embodiments, primers may be associated with a solid support. In certain embodiments, target nucleic acid (e.g., template nucleic acid or target sequences) may be associated with a solid support. A nucleic acid (primer or target) in association with a solid support often is referred to as a solid phase nucleic acid.

In some embodiments, nucleic acid molecules provided for amplification are in a “microreactor”. As used herein, the term “microreactor” refers to a partitioned space in which a nucleic acid molecule can hybridize to a solid support nucleic acid molecule. Examples of microreactors include, without limitation, an emulsion globule (described hereafter) and a void in a substrate. A void in a substrate can be a pit, a pore or a well (e.g., microwell, nanowell, picowell, micropore, or nanopore) in a substrate constructed from a solid material useful for containing fluids (e.g., plastic (e.g., polypropylene, polyethylene, polystyrene) or silicon) in certain embodiments. Emulsion globules are partitioned by an immiscible phase as described in greater detail hereafter. In some embodiments, the microreactor volume is large enough to accommodate one solid support (e.g., bead) in the microreactor and small enough to exclude the presence of two or more solid supports in the microreactor.

The term “emulsion” as used herein refers to a mixture of two immiscible and unblendable substances, in which one substance (the dispersed phase) often is dispersed in the other substance (the continuous phase). The dispersed phase can be an aqueous solution (i.e., a solution comprising water) in certain embodiments. In some embodiments, the dispersed phase is composed predominantly of water (e.g., greater than 70%, greater than 75%, greater than 80%, greater than 85%, greater than 90%, greater than 95%, greater than 97%, greater than 98% and greater than 99% water (by weight)). Each discrete portion of a dispersed phase, such as an aqueous dispersed phase, is referred to herein as a “globule” or “microreactor.” A globule sometimes may be spheroidal, substantially spheroidal or semi-spheroidal in shape, in certain embodiments.

The terms “emulsion apparatus” and “emulsion component(s)” as used herein refer to apparatus and components that can be used to prepare an emulsion. Non-limiting examples of emulsion apparatus include without limitation counter-flow, cross-current, rotating drum and membrane apparatus suitable for use by a person of ordinary skill to prepare an emulsion. An emulsion component forms the continuous phase of an emulsion in certain embodiments, and includes without limitation a substance immiscible with water, such as a component comprising or consisting essentially of an oil (e.g., a heat-stable, biocompatible oil (e.g., light mineral oil)). A biocompatible emulsion stabilizer can be utilized as an emulsion component. Emulsion stabilizers include without limitation Atlox 4912, Span 80 and other biocompatible surfactants.

In some embodiments, components useful for biological reactions can be included in the dispersed phase. Globules of the emulsion can include (i) a solid support unit (e.g., one bead or one particle); (ii) sample nucleic acid molecule; and (iii) a sufficient amount of extension agents to elongate solid phase nucleic acid and amplify the elongated solid phase nucleic acid (e.g., extension nucleotides, polymerase, primer). In some embodiments, endonucleases and components necessary for endonuclease function may be included in the components useful for biological reactions as described below in the example section. Inactive globules in the emulsion may include a subset of these components (e.g., solid support and extension reagents and no sample nucleic acid) and some can be empty (i.e., some globules will include no solid support, no sample nucleic acid and no extension agents).

Emulsions may be prepared using known suitable methods (e.g., Nakano et al. “Single-molecule PCR using water-in-oil emulsion;” Journal of Biotechnology 102 (2003) 117-124). Emulsification methods include without limitation adjuvant methods, counter-flow methods, cross-current methods, rotating drum methods, membrane methods, and the like. In certain embodiments, an aqueous reaction mixture containing a solid support (hereafter the “reaction mixture”) is prepared and then added to a biocompatible oil. In certain embodiments, the reaction mixture may be added dropwise into a spinning mixture of biocompatible oil (e.g., light mineral oil (Sigma)) and allowed to emulsify. In some embodiments, the reaction mixture may be added dropwise into a cross-flow of biocompatible oil. The size of aqueous globules in the emulsion can be adjusted, such as by varying the flow rate and speed at which the components are added to one another, for example.

The size of emulsion globules can be selected by the person of ordinary skill in certain embodiments based on two competing factors: (i) globules are sufficiently large to encompass one solid support molecule, one sample nucleic acid molecule, and sufficient extension agents for the degree of elongation and amplification required; and (ii) globules are sufficiently small so that a population of globules can be amplified by conventional laboratory equipment (e.g., thermocycling equipment, test tubes, incubators and the like). Globules in the emulsion can have a nominal, mean or average diameter of about 5 microns to about 500 microns, about 10 microns to about 350 microns, about 50 to 250 microns, about 100 microns to about 200 microns, or about 5, 10, 15, 20, 25, 30, 35, 40, 45, 50, 55, 60, 65, 70, 75, 80, 85, 90, 95, 100, 200, 300, 400 or 500 microns in certain embodiments.

In certain embodiments, amplified nucleic acid species in a set are of identical length, and sometimes the amplified nucleic acid species in a set are of a different length. For example, one amplified nucleic acid species may be longer than one or more other amplified nucleic acid species in the set by about 1 to about 100 nucleotides (e.g., about 2, 3, 4, 5, 6, 7, 8, 9, 10, 11, 12, 13, 14, 15, 16, 17, 18, 19, 20, 30, 40, 50, 60, 70, 80 or 90 nucleotides longer).

In some embodiments, a ratio can be determined for the amount of one amplified nucleic acid species in a set to the amount of another amplified nucleic acid species in the set (hereafter a “set ratio”). In some embodiments, the amount of one amplified nucleic acid species in a set is about equal to the amount of another amplified nucleic acid species in the set (i.e., amounts of amplified nucleic acid species in a set are about 1:1), which generally is the case when the number of chromosomes or the amount of DNA representative of nucleic acid species in a sample bearing each nucleotide sequence species amplified is about equal. The term “amount” as used herein with respect to amplified nucleic acid species refers to any suitable measurement, including, but not limited to, copy number, weight (e.g., grams) and concentration (e.g., grams per unit volume (e.g., milliliter); molar units). In some embodiments, the ratio of fetal nucleic acid to maternal nucleic acid (or conversely maternal nucleic acid to fetal nucleic acid) can be used in conjunction with measurements of the ratios of mismatch sequences for determination of chromosomal abnormalities possibly associated with sex chromosomes. That is, the percentage of fetal nucleic acid detected in a maternal nucleic acid background or the ratio of fetal to maternal nucleic acid in a sample, can be used to detect chromosomal aneuploidies.

In certain embodiments, the amount of one amplified nucleic acid species in a set can differ from the amount of another amplified nucleic acid species in a set, even when the number of chromosomes in a sample bearing each nucleotide sequence species amplified is about equal. In some embodiments, amounts of amplified nucleic acid species within a set may vary up to a threshold level at which a chromosome abnormality can be detected with a confidence level of about 95% (e.g., about 90, 91, 92, 93, 94, 95, 96, 97, 98, 99, or greater than 99%). In certain embodiments, the amounts of the amplified nucleic acid species in a set vary by about 50% or less (e.g., about 45, 40, 35, 30, 25, 20, 15, 10, 5, 4, 3, 2 or 1%, or less than 1%). Thus, in certain embodiments amounts of amplified nucleic acid species in a set may vary from about 1:1 to about 1:1.5. Without being limited by theory, certain factors can lead to the observation that the amount of one amplified nucleic acid species in a set can differ from the amount of another amplified nucleic acid species in a set, even when the number of chromosomes in a sample bearing each nucleotide sequence species amplified is about equal. Such factors may include different amplification efficiency rates and/or amplification from a chromosome not intended in the assay design.

Each amplified nucleic acid species in a set generally is amplified under conditions that amplify that species at a substantially reproducible level. The term “substantially reproducible level” as used herein refers to consistency of amplification levels for a particular amplified nucleic acid species per unit template nucleic acid (e.g., per unit template nucleic acid that contains the particular nucleotide sequence species amplified). A substantially reproducible level varies by about 1% or less in certain embodiments, after factoring the amount of template nucleic acid giving rise to a particular amplification nucleic acid species (e.g., normalized for the amount of template nucleic acid). In some embodiments, a substantially reproducible level varies by 5%, 4%, 3%, 2%, 1.5%, 1%, 0.5%, 0.1%, 0.05%, 0.01%, after factoring the amount of template nucleic acid giving rise to a particular amplification nucleic acid species.

In some embodiments amplification nucleic acid species (e.g., amplified target sequences) of oligonucleotide species composition sets described herein may be generated in one reaction vessel. In some embodiments amplification of mismatch sequences may be performed in a single reaction vessel. In certain embodiments, mismatch sequences (on the same or different chromosomes) may be amplified by a single oligonucleotide species pair or set. In some embodiments target sequences may be amplified by a single oligonucleotide species pair or set. In some embodiments target sequences in a set may be amplified with two or more oligonucleotide species pairs. In some embodiments a subsequence of a target nucleic acid may be amplified using a single oligonucleotide species pair or set. In some embodiments a subsequence of a target nucleic acid may be amplified using two or more oligonucleotide species pairs.

Oligonucleotides

Oligonucleotide species described herein are useful for amplification, detection, quantification and sequencing of target nucleic acids. An oligonucleotide species composition may include one or more types of oligonucleotides. In some embodiments oligonucleotide species may be complementary to, and hybridize or anneal specifically to or near (e.g., adjacent to) sequences that flank a target region therein. In some embodiments the oligonucleotide species described herein are used in sets, where a set contains at least a pair. In some embodiments a set of oligonucleotide species may include a third or a fourth nucleic acid (e.g., two pairs of oligonucleotide species or nested sets of oligonucleotide species, for example). A plurality of oligonucleotide species pairs may constitute a primer set in certain embodiments (e.g., about 2, 3, 4, 5, 6, 7, 8, 9, 10, 15, 20, 25, 30, 35, 40, 45, 50, 55, 60, 65, 70, 75, 80, 85, 90, 95 or 100 pairs). In some embodiments a plurality of oligonucleotide species sets, each set comprising pair(s) of primers, may be used.

The term “oligonucleotide species” as used herein refers to a nucleic acid that comprises a nucleotide sequence capable of hybridizing or annealing to a target nucleic acid, at or near (e.g., adjacent to) a specific region of interest. As used herein, the term “PCR oligonucleotide species(s)” refers to oligonucleotides that can be used in a polymerase chain reaction (PCR) to amplify a target nucleotide sequence, for example. In certain embodiments, at least one of the PCR oligonucleotide species for amplification of a nucleotide sequence encoding a target nucleic acid can be a sequence-specific oligonucleotide species. In some embodiments, oligonucleotide species described herein may be modified (e.g., addition of a universal primer sequence) to improve multiplexing.

Oligonucleotide species described herein can allow for specific determination of a target nucleic acid nucleotide sequence or detection of the target nucleic acid sequence (e.g., presence or absence of a sequence or copy number of a sequence), or feature thereof, for example.

Oligonucleotide species described herein may also be used to detect amplification products or extension products, in certain embodiments. The oligonucleotide compositions and methods of use described herein are useful for minimizing or eliminating extension and/or amplification artifacts (e.g., “primer-dimers” and artifacts caused by annealing and extension during temperature transitions in a PCR thermocycling profile, for example) that can sometimes occur in nucleic acid extension or amplification based assays. The oligonucleotide species described herein include endonuclease cleavage sites for thermostable endonucleases that can be used in methods (single tube assays, multiplexed assays and the like), also described herein, that combine hybridization, cleavage and extension or amplification conditions to allow specific target identification and/or amplification.

The oligonucleotide species described herein are often synthetic, but naturally occurring nucleic acid sequences with similar structure and/or function may be used, in some embodiments. The term “specific”, “specifically” or “specificity”, as used herein with respect to nucleic acids, refers to the binding or hybridization of one molecule to another molecule, such as a primer for a target polynucleotide sequence. That is, “specific”, “specifically” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “oligonucleotide species”, “oligonucleotide species”, “oligonucleotide composition”, “primer”, “oligo”, or “oligonucleotide” may be used interchangeably throughout the document, when referring to primers.

Oligonucleotide species described herein may be modified. For example, oligonucleotide species may be modified to decrease their length and/or increase their specificity. In some embodiments, one ore more duplex stabilizers (e.g., minor groove binders, spermidine or acridine) are incorporated into the oligonucleotide species. Minor groove binders are further described in U.S. Pat. Nos. 5,801,155; 6,127,121; 6,312,894; and 6,426,408.

Oligonucleotide species described herein can be designed and synthesized using suitable processes, and may be of any length suitable for hybridizing to a nucleotide sequence of interest (e.g., where the nucleic acid is in liquid phase or bound to a solid support) and performing analysis processes described herein. Oligonucleotide species described herein may be designed based upon a target nucleotide sequence.

The terms “oligonucleotide” and “polynucleotide” as used herein each refer to nucleic acids, and can be of any suitable length. An oligonucleotide species, or polynucleotide, in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. In some embodiments, an oligonucleotide or polynucleotide is about 18 to about 27 nucleotides in length. An oligonucleotide species may be composed of naturally occurring and/or non-naturally occurring nucleotides (e.g., labeled nucleotides), or a mixture thereof. Oligonucleotide species embodiments suitable for use with method embodiments described herein may be synthesized and labeled using known techniques. Oligonucleotides and polynucleotides (e.g., primers) may be chemically synthesized according to the solid phase phosphoramidite triester method first described by Beaucage and Caruthers, Tetrahedron Letts., 22:1859-1862, 1981, using an automated synthesizer, as described in Needham-VanDevater et al., Nucleic Acids Res. 12:6159-6168, 1984. Purification of oligonucleotides can be effected by native acrylamide gel electrophoresis or by anion-exchange high-performance liquid chromatography (HPLC), for example, as described in Pearson and Regnier, J. Chrom., 255:137-149, 1983. Oligonucleotide species containing abasic AP endonuclease cleavage sites can be synthesized according to World Wide Web URL glenresearch.com//GlenReports/GR14-13.html, for example.

All or a portion of an oligonucleotide species nucleic acid sequence (naturally occurring or synthetic) may be substantially complementary to a target nucleic acid sequence, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are regions of counterpart, target and capture nucleotide sequences 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Oligonucleotide compositions that contain subsequences that are substantially complimentary to a target nucleic acid sequence are also substantially identical to the compliment of the target nucleic acid sequence. That is, primers can be substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Oligonucleotide species sequences and length may affect hybridization to target nucleic acid sequences. Depending on the degree of mismatch between the oligonucleotide species and target nucleic acid, low, medium or high stringency conditions may be used to effect oligonucleotide/target annealing. As used herein, the term “stringent conditions” refers to conditions for hybridization and washing. Methods for hybridization reaction temperature condition optimization are known to those of skill in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

In embodiments using extension or amplification methods described herein, “stringent conditions” can also refer to conditions under which an intact oligonucleotide species can anneal to a target nucleic acid, but where one or more cleaved fragments of the oligonucleotide species cannot anneal to the target nucleic acid (e.g., intact oligonucleotide anneals at 65 C and one or more fragments anneals at 50 C). In some embodiments, the “stringent conditions” for extension and/or amplification methods described herein are; substantially similar to, a subset of, or include as a subset, hybridization conditions, cleavage conditions, extension conditions, amplification conditions or combinations thereof.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of an oligonucleotide species, to a nucleic acid molecule having a sequence complementary to the oligonucleotide species compared to hybridization to a nucleic acid molecule not having a complementary sequence. For example, specific hybridization includes the hybridization of an oligonucleotide species to a target nucleic acid sequence that is complementary to at least a portion of the oligonucleotide species.

In some embodiments oligonucleotide species can include a nucleotide subsequence that may be complementary to a solid phase nucleic acid oligonucleotide hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). An oligonucleotide species may contain a nucleotide subsequence not complementary to or not substantially complementary to a solid phase nucleic acid oligonucleotide hybridization sequence (e.g., at the 3′ or 5′ end of the nucleotide subsequence in the oligonucleotide species complementary to or substantially complementary to the solid phase oligonucleotide hybridization sequence).

An oligonucleotide species, in certain embodiments, may contain a detectable feature, moiety, molecule or entity (e.g., a fluorophore, radioisotope, colorimetric agent, particle, enzyme and the like). In some embodiments, a detectable feature may be a capture agent or a blocking agent. In some embodiments each oligonucleotide species may contain a blocking moiety. In some embodiments the blocking moiety of a first oligonucleotide species is different than the blocking moiety of a second oligonucleotide species. Non-limiting examples of blocking agents include; phosphate group, thiol group, phosphorothioate group, amino modifier, biotin, biotin-TEG, cholesteryl-TEG, digoxigenin NHS ester, thiol modifier C3 S—S (Disulfide), inverted dT, C3 spacer and the like. In some embodiments more than one blocking group can be incorporated into an oligonucleotide species at, or near, one more endonuclease cleavage sites to allow the oligonucleotide species to be sequentially deblocked to allow multiple rounds of extension. When desired, the nucleic acid can be modified to include a detectable feature or blocking moiety using any method known to one of skill in the art. The feature may be incorporated as part of the synthesis, or added on prior to using the oligonucleotide species in any of the processes described herein. Incorporation of a detectable feature may be performed either in liquid phase or on solid phase. In some embodiments the detectable feature may be useful for detection of targets. In some embodiments the detectable feature may be useful for the quantification target nucleic acids (e.g., determining copy number of a particular sequence or species of nucleic acid). Any detectable feature suitable for detection of an interaction or biological activity in a system can be appropriately selected and utilized by the artisan. Examples of detectable features are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., 125I, 131I, 35S, 31P, 32P, 33P, 14C, 3H, 7Be, 28Mg, 57Co, 65Zn, 67Cu, 68Ge, 82Sr, 83Rb, 95Tc, 96Tc, 103Pd, 109Cd, and 127Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and other cofactors or biomolecules such as digoxigenin, strepdavidin, biotin (e.g., members of a binding pair such as biotin and avidin for example), affinity capture moieties, 3′ blocking agents (e.g., phosphate group, thiol group, phosphorothioate, amino modifier, biotin, biotin-TEG, cholesteryl-TEG, digoxigenin NHS ester, thiol modifier C3 S—S (Disulfide), inverted dT, C3 spacer) and the like. In some embodiments an oligonucleotide species may be labeled with an affinity capture moiety. Also included in detectable features are those labels useful for mass modification for detection with mass spectrometry (e.g., matrix-assisted laser desorption ionization (MALDI) mass spectrometry and electrospray (ES) mass spectrometry).

An oligonucleotide species also may refer to a polynucleotide sequence that hybridizes to a subsequence of a target nucleic acid or another oligonucleotide species and facilitates the detection of an oligonucleotide, a target nucleic acid or both, and amplification products or extension products, as with molecular beacons, for example. The term “molecular beacon” as used herein refers to detectable molecule, wherein the detectable feature, or property, of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In some embodiments a molecular beacon can be a single-stranded oligonucleotide capable of forming a stem-loop structure, where the loop sequence may be complementary to a target nucleic acid sequence of interest and is flanked by short complementary arms that can form a stem. The oligonucleotide may be labeled at one end with a fluorophore and at the other end with a quencher molecule. In the stem-loop conformation, energy from the excited fluorophore is transferred to the quencher, through long-range dipole-dipole coupling similar to that seen in fluorescence resonance energy transfer, or FRET, and released as heat instead of light. When the loop sequence is hybridized to a specific target sequence, the two ends of the molecule are separated and the energy from the excited fluorophore is emitted as light, generating a detectable signal. Molecular beacons offer the added advantage that removal of excess probe is unnecessary due to the self-quenching nature of the unhybridized probe. In some embodiments molecular beacon probes can be designed to either discriminate or tolerate mismatches between the loop and target sequences by modulating the relative strengths of the loop-target hybridization and stem formation. As referred to herein, the term “mismatched nucleotide” or a “mismatch” refers to a nucleotide that is not complementary to the target sequence at that position or positions. A probe may have at least one mismatch, but can also have 2, 3, 4, 5, 6 or 7 or more mismatched nucleotides.

In some embodiments the oligonucleotide species described herein can contain internal subsequences that may form stem-loop structures, where the stem-loop sequences are not complementary to any sequence in the template DNA. The Tm of the internal structure is too low for it to form a stem-loop structure, unless the two sides are brought together by the annealing of the 5′ and 3′ ends to the template (e.g., the reverse of a molecular beacon).

In certain embodiments, oligonucleotide species in a composition can be designed so that they specifically hybridize to a particular target nucleic acid allele. For example, a composition may include two oligonucleotides that differ by only one base pair (e.g., adenine at a position in one oligonucleotide species and cytosine in another species at the same position), and thereby hybridize specifically to each of two alleles that contain a thymine or guanine at the same position. Such oligonucleotide species compositions are useful for detecting particular single nucleotide polymorphism variants in a nucleic acid composition. In some embodiments, a variant nucleotide in oligonucleotide species is located at or near the middle of each oligonucleotide.

Detection

A detectable feature (e.g., mass, signal emission, sequence) of polynucleotide sequences generated, amplified nucleic acid species (e.g. amplicons or amplification products), detectable products (e.g., extension products, cleavage products, cleavage fragments) and polymorphisms, prepared from the foregoing, can be detected by a suitable detection process. Non limiting examples of methods of detection, quantification, sequencing and the like are: mass detection of mass modified amplicons (e.g., matrix-assisted laser desorption ionization (MALDI) mass spectrometry and electrospray (ES) mass spectrometry), a primer extension method (e.g., iPLEXTM; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension (e.g., microarray sequence determination methods), Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization methods (e.g., hybridization using at least one probe, hybridization using at least one fluorescently labeled probe, and the like), conventional dot blot analyses, single strand conformational polymorphism analysis (SSCP, e.g., U.S. Pat. Nos. 5,891,625 and 6,013,499; Orita et al., Proc. Natl. Acad. Sci. U.S.A 86: 27776-2770 (1989)), denaturing gradient gel electrophoresis (DGGE), heteroduplex analysis, mismatch cleavage detection, and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993), cloning and sequencing, electrophoresis, the use of hybridization probes and quantitative real time polymerase chain reaction (QRT-PCR), digital PCR, nanopore sequencing, chips and combinations thereof. Also, contacting amplification products with an intercalating agent (e.g., asymmetrical cyanine dye (e.g., SYBR

Green agent)), and detecting the amount of intercalating agent (e.g., detecting the agent over time), can be utilized to detect amplification products and cleavage products generated there from. The detection and quantification of alleles or paralogs can be carried out using the “closed-tube” methods described in U.S. patent application Ser. No. 11/950,395, which was filed Dec. 4, 2007. In some embodiments the amount of each amplified nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

In addition to the methods of detection listed above, the following detection methods may also be used to detect amplified nucleic acid species (e.g., target sequences). In some embodiments, the amplified nucleic acid species can be sequenced directly using any suitable nucleic acid sequencing method. Non-limiting examples of nucleic acid sequencing methods useful for process described herein are; pyrosequencing, nanopore based sequencing methods (e.g., sequencing by synthesis), sequencing by ligation, sequencing by hybridization, microsequencing (primer extension based polymorphism detection), and conventional nucleotide sequencing (e.g., dideoxy sequencing using conventional methods).

In some embodiments, the amplified sequence(s) may be cloned prior to sequence analysis. That is, the amplified nucleic acid species may be ligated into a nucleic acid cloning vector by any process known to one of skill in the art. Cloning of the amplified nucleic acid species may be performed by including unique restriction sites in oligonucleotide species subsequences, which can be used to generate a fragment flanked by restriction sites useful for cloning into an appropriately prepared vector, in some embodiments. In certain embodiments blunt-ended cloning can be used to clone amplified nucleic acid species into an appropriately prepared cloning vector. Cloning of the amplified nucleic acid species may be useful for further manipulation, modification, storage, and analysis of the target sequence of interest. In some embodiments, oligonucleotide species compositions may be designed to overlap an SNP site to allow analysis by allele-specific PCR. Allele-specific PCR may be used to discriminate between nucleic acids in a nucleic acid composition (e.g., fetal target in nucleic acid isolated from maternal sample, for example), because only the correctly hybridized primers will be amplified. In some embodiments, the amplified nucleic acid species may be further analyzed by hybridization (e.g., liquid or solid phase hybridization using sequence specific probes, for example).

Amplified nucleic acids (including amplified nucleic acids that result from reverse transcription) may be modified nucleic acids. Reverse transcribed nucleic acids also may be modified nucleic acids. Modified nucleic acids can include nucleotide analogs, and in certain embodiments include a detectable feature and/or a capture agent (e.g., biomolecules or members of a binding pair, as listed below). In some embodiments the detectable feature and the capture agent can be the same moiety. Modified nucleic acids can be detected by detecting a detectable feature or “signal-generating moiety” in some embodiments. The term “signal-generating” as used herein refers to any atom or molecule that can provide a detectable or quantifiable effect, and that can be attached to a nucleic acid. In certain embodiments, a detectable feature generates a unique light signal, a fluorescent signal, a luminescent signal, an electrical property, a chemical property, a magnetic property and the like.

Detectable features include, but are not limited to, nucleotides (labeled or unlabelled), compomers, sugars, peptides, proteins, antibodies, chemical compounds, conducting polymers, binding moieties such as biotin, mass tags, colorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) and the like, some of which are further described below. In some embodiments a probe or oligonucleotide species may contain a signal-generating moiety that hybridizes to a target and alters the passage of the target nucleic acid through a nanopore, and can generate a signal when released from the target nucleic acid when it passes through the nanopore (e.g., alters the speed or time through a pore of known size).

A solution containing amplicons produced by an amplification process, or a solution containing extension products produced by an extension process, can be subjected to further processing. For example, a solution can be contacted with an agent that removes phosphate moieties from free nucleotides that have not been incorporated into an amplicon or extension product. An example of such an agent is a phosphatase (e.g., alkaline phosphatase). Amplicons and extension products also may be associated with a solid phase, may be washed, may be contacted with an agent that removes a terminal phosphate (e.g., exposure to a phosphatase), may be contacted with an agent that removes a terminal nucleotide (e.g., exonuclease), may be contacted with an agent that cleaves (e.g., endonuclease, ribonuclease), and the like.

The term “solid support” or “solid phase” as used herein refers to an insoluble material with which nucleic acid can be associated. Examples of solid supports for use with processes described herein include, without limitation, arrays, beads (e.g., paramagnetic beads, magnetic beads, microbeads, nanobeads) and particles (e.g., microparticles, nanoparticles). Particles or beads having a nominal, average or mean diameter of about 1 nanometer to about 500 micrometers can be utilized, such as those having a nominal, mean or average diameter, for example, of about 10 nanometers to about 100 micrometers; about 100 nanometers to about 100 micrometers; about 1 micrometer to about 100 micrometers; about 10 micrometers to about 50 micrometers; about 1, 5, 10, 15, 20, 25, 30, 35, 40, 45, 50, 55, 60, 65, 70, 75, 80, 85, 90, 95, 100, 200, 300, 400, 500, 600, 700, 800 or 900 nanometers; or about 1, 5, 10, 15, 20, 25, 30, 35, 40, 45, 50, 55, 60, 65, 70, 75, 80, 85, 90, 95, 100, 200, 300, 400, 500 micrometers.

A solid support can comprise virtually any insoluble or solid material, and often a solid support composition is selected that is insoluble in water. For example, a solid support can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)) and the like. Beads or particles may be swellable (e.g., polymeric beads such as Wang resin) or non-swellable (e.g., CPG). Commercially available examples of beads include without limitation Wang resin, Merrifield resin and Dynabeads® and SoluLink.

A solid support may be provided in a collection of solid supports. A solid support collection comprises two or more different solid support species. The term “solid support species” as used herein refers to a solid support in association with one particular solid phase nucleic acid species or a particular combination of different solid phase nucleic acid species. In certain embodiments, a solid support collection comprises 2 to 10,000 solid support species, 10 to 1,000 solid support species or about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 unique solid support species. The solid supports (e.g., beads) in the collection of solid supports may be homogeneous (e.g., all are Wang resin beads) or heterogeneous (e.g., some are Wang resin beads and some are magnetic beads). Each solid support species in a collection of solid supports sometimes is labeled with a specific identification tag. An identification tag for a particular solid support species sometimes is a nucleic acid (e.g., “solid phase nucleic acid”) having a unique sequence in certain embodiments. An identification tag can be any molecule that is detectable and distinguishable from identification tags on other solid support species.

Mass spectrometry is a particularly effective method for the detection of nucleic acids (e.g., PCR amplicon, primer extension product, detector probe cleaved from a target nucleic acid). Presence of a target nucleic acid is verified by comparing the mass of the detected signal with the expected mass of the target nucleic acid. The relative signal strength, e.g., mass peak on a spectra, for a particular target nucleic acid indicates the relative population of the target nucleic acid amongst other nucleic acids, thus enabling calculation of a ratio of target to other nucleic acid or sequence copy number directly from the data. For a review of genotyping methods using Sequenom® standard iPLEX™ assay and MassARRAY® technology, see Jurinke, C., Oeth, P., van den Boom, D., “MALDI-TOF mass spectrometry: a versatile tool for high-performance DNA analysis.” Mol. Biotechnol. 26, 147-164 (2004); and Oeth, P. et al., “iPLEX™ Assay: Increased Plexing Efficiency and Flexibility for MassARRAY® System through single base primer extension with mass-modified Terminators.” SEQUENOM Application Note (2005). For a review of detecting and quantifying target nucleic using cleavable detector probes (e.g., oligonucleotide compositions described herein) that are cleaved during the amplification process and detected by mass spectrometry, see U.S. patent application Ser. No. 11/950,395, which was filed Dec. 4, 2007, and is hereby incorporated by reference. Such approaches may be adapted to detection of chromosome abnormalities using oligonucleotide species compositions and methods described herein.

In some embodiments, amplified nucleic acid species may be detected by (a) contacting the amplified nucleic acid species (e.g., amplicons) with extension oligonucleotide species compositions (e.g., detection or detector oligonucleotides or primers), (b) preparing extended extension oligonucleotide species compositions, and (c) determining the relative amount of the one or more mismatch nucleotides (e.g., SNP that exist between SNP-alleles or paralogous sequences) by analyzing the extended detection oligonucleotide species compositions (e.g., extension oligonucleotides, or detection of extension products). In certain embodiments one or more mismatch nucleotides may be analyzed by mass spectrometry. In some embodiments amplification, using methods described herein, may generate between about 1 to about 100 amplicon sets, about 2 to about 80 amplicon sets, about 4 to about 60 amplicon sets, about 6 to about 40 amplicon sets, and about 8 to about 20 amplicon sets (e.g., about 1, 2, 3, 4, 5, 6, 7, 8, 9, 10, 15, 20, 25, 30, 35, 40, 45, 50, 55, 60, 65, 70, 75, 80, 85, 90, 95, or about 100 amplicon sets).

An example using mass spectrometry for detection of amplicon sets (e.g., sets of amplification products) is presented herein. Amplicons may be contacted (in solution or on solid phase) with a set of oligonucleotides (the same oligonucleotide species compositions used for amplification or different oligonucleotides representative of subsequences in the oligo or target nucleic acid) under hybridization conditions, where: (1) each oligonucleotide in the set comprises a hybridization sequence capable of specifically hybridizing to one amplicon under the hybridization conditions when the amplicon is present in the solution, (2) each oligonucleotide in the set comprises a distinguishable tag located 5′ of the hybridization sequence, (3) a feature of the distinguishable tag of one oligonucleotide detectably differs from the features of distinguishable tags of other oligonucleotides in the set; and (4) each distinguishable tag specifically corresponds to a specific amplicon and thereby specifically corresponds to a specific target nucleic acid. The hybridized amplicon and “detection” oligonucleotide species are subjected to nucleotide synthesis conditions that allow extension of the detection oligonucleotide by one or more nucleotides (labeled with a detectable entity or moiety, or unlabeled), where one of the one or more nucleotides can be a terminating nucleotide. In some embodiments one or more of the nucleotides added to the oligonucleotide species may comprises a capture agent. In embodiments where hybridization occurred in solution, capture of the oligo/amplicon to solid support may be desirable. The detectable moieties or entities can be released from the extended detection oligonucleotide species composition, and detection of the moiety determines the presence, absence, copy number of the nucleotide sequence of interest, or in some embodiments can provide information regarding the status of a reaction. In certain embodiments, the extension may be performed once yielding one extended oligonucleotide. In some embodiments, the extension may be performed multiple times (e.g., under amplification conditions) yielding multiple copies of the extended oligonucleotide. In some embodiments performing the extension multiple times can produce a sufficient number of copies such that interpretation of signals, representing copy number of a particular sequence, can be made with a confidence level of 95% or more (e.g., confidence level of 95% or more, 96% or more, 97% or more, 98% or more, 99% or more, or a confidence level of 99.5% or more). In some embodiments, the method for detecting amplicon sets can be used to detect extension products.

Methods provided herein allow for high-throughput detection of nucleic acid species in a plurality of nucleic acids (e.g., nucleotide sequence species, amplified nucleic acid species and detectable products generated from the foregoing). Multiplexing refers to the simultaneous amplification, and/or detection of the presence or absence, of more than one nucleic acid species. General methods for performing multiplexed reactions in conjunction with mass spectrometry are known (see, e.g., U.S. Pat. Nos. 6,043,031, 5,547,835 and International PCT application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly automated processes for analyzing sequence variations with high speed and accuracy, in some embodiments. In certain embodiments, methods herein may be multiplexed at high levels in a single reaction.

Microarrays may be adapted for use with oligonucleotide species compositions and method embodiments described herein. A microarray can be utilized for determining whether a polymorphic variant is present or absent in a nucleic acid sample. A microarray may include any oligonucleotides species compositions described herein, and methods for making and using oligonucleotide microarrays suitable for prognostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic target nucleic acid site. Microarrays may be used with multiplexed protocols described herein.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301-303, 303-305, 305-307, 307-309, 309-311, 311-313, 313-315, 315-317, 317-319, 319-321, 321-323, 323-325, 325-327, 327-329, 329-331, 331-333, 333- 335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401- 403, 403-405, 405-407, 407-409, 409-411, 411-413, 413-415, 415-417, 417-419, 419-421, 421-423, 423-425, 425-427, 427-429, 429-431, 431-433, 433- 435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays often include primer and oligonucleotide species composition design methods and reaction design methods. For primer and oligonucleotide species composition design in multiplexed assays, the same general guidelines for oligonucleotide species composition design applies for uniplexed reactions. The oligonucleotide species compositions described herein are designed to minimize or eliminate artifacts, thus avoiding false priming and primer dimers, the only difference being more oligonucleotides species are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

Nucleotide sequence species, amplified nucleic acid species, or detectable products generated from the foregoing may be subject to sequence analysis. The term “sequence analysis” as used herein refers to determining a nucleotide sequence of an extension or amplification product. The entire sequence or a partial sequence of an extension or amplification product can be determined, and the determined nucleotide sequence is referred to herein as a “read.” For example, one-time “primer extension” products or linear amplification products may be analyzed directly without further amplification in some embodiments (e.g., by using single-molecule sequencing methodology (described in greater detail hereafter)). In certain embodiments, linear amplification products may be subject to further amplification and then analyzed (e.g., using sequencing by ligation or pyrosequencing methodology (described in greater detail hereafter)). Reads may be subject to different types of sequence analysis. Any suitable sequencing method can be utilized to detect, and determine the amount of, nucleotide sequence species, amplified nucleic acid species, or detectable products generated from the foregoing. Examples of certain sequencing methods are described hereafter.

The terms “sequence analysis apparatus” and “sequence analysis component(s)” used herein refer to apparatus, and one or more components used in conjunction with such apparatus, that can be used by a person of ordinary skill to determine a nucleotide sequence from amplification products resulting from processes described herein (e.g., linear and/or exponential amplification products). Examples of sequencing platforms include, without limitation, the 454 platform (Roche) (Margulies, M. et al. 2005 Nature 437, 376-380), IIlumina Genomic Analyzer (or Solexa platform) or SOLID System (Applied Bios stems) or the Helicos True Single Molecule DNA sequencing technology (Harris TD et al. 2008 Science, 320, 106-109), the single molecule, real-time (SMRT™) technology of Pacific Biosciences, and nanopore sequencing (Soni GV and Meller A. 2007 Clin Chem 53: 1996-2001). Such platforms allow sequencing of many nucleic acid molecules isolated from a specimen at high orders of multiplexing in a parallel manner (Dear Brief Funct Genomic Proteomic 2003; 1: 397-416). Each of these platforms allows sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing. Nucleotide sequence species, amplification nucleic acid species and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated oligonucleotide species, preparing the oligonucleotide for another round of ligation. In some embodiments oligonucleotide species compositions may be labeled with more than one fluorescent label (e.g., 1 fluorescent label, 2, 3, or 4 fluorescent labels).

An example of a system that can be used by a person of ordinary skill based on sequencing by ligation generally involves the following steps. Clonal bead populations can be prepared in emulsion microreactors containing target nucleic acid sequences (“template”), amplification reaction components (e.g., including cleavage reaction components where applicable), beads and oligonucleotide species compositions described herein. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four-color dye-labeled probes competes for ligation to the sequencing oligonucleotide species. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Oligonucleotide species reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Target nucleic acids may be immobilized to a solid support, hybridized with a sequencing oligonucleotide species (e.g., oligonucleotide species compositions described herein, for example), incubated with DNA polymerase, an appropriate endonuclease, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. The amount of light generated is proportional to the number of bases added. Accordingly, the sequence downstream of the sequencing oligonucleotide species can be determined.

An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing an oligonucleotide species to a target nucleic acid sequence to generate a complex; associating the complex with a solid phase; iteratively extending the oligonucleotide species by a nucleotide tagged with a fluorescent molecule; and capturing an image of fluorescence resonance energy transfer signals after each iteration (e.g., U.S. Pat. No. 7,169,314; Braslaysky et al., PNAS 100(7): 3960-3964 (2003)). Such a system can be used to directly sequence amplification products (linearly or exponentially amplified products) generated by processes described herein. In some embodiments the amplification products can be hybridized to an oligonucleotide that contains sequences complementary to immobilized capture sequences present on a solid support, a bead or glass slide for example. Hybridization of the oligonucleotide species -amplification product complexes with the immobilized capture sequences, immobilizes amplification products to solid supports for single pair FRET based sequencing by synthesis. The oligonucleotide species often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the oligonucleotide species -amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting target nucleic acid and solid support under conditions in which a single molecule of sample nucleic acid hybridizes to a single molecule of a solid support. Such conditions can include providing the solid support molecules and a single molecule of target nucleic acid in a “microreactor.” Such conditions also can include providing a mixture in which the target nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a target nucleic acid for sequencing (“base nucleic acid,” e.g., linked probe molecule) with sequence-specific detectors (e.g., oligonucleotide species compositions described herein), under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. In some embodiments a detector can be an oligonucleotide species composition having an internal stem-loop that can function as a detectable feature when cleaved from the intact oligonucleotide species composition, as described herein. A detector often comprises one or more detectable features independently selected from those described herein. Each detectable feature or label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons sometimes are performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis can be facilitated by the use of sequence analysis apparatus and components described above.

Target nucleic acid sequences also can be detected using standard electrophoretic techniques. Although the detection step can sometimes be preceded by an amplification step, amplification is not required in the embodiments described herein. Examples of methods for detection and quantification of target nucleic acid sequences using electrophoretic techniques can be found in the art. A non-limiting example is presented herein. After running a sample (e.g., mixed nucleic acid sample isolated from maternal serum, or amplification nucleic acid species, for example) in an agarose or polyacrylamide gel, the gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the band, thus detecting and quantifying the target sequence of interest. In some embodiments, restriction enzymes capable of distinguishing between maternal and paternal alleles may be used to detect and quantify target nucleic acid species. In certain embodiments, oligonucleotide species compositions specific to target nucleic acids (e.g., a specific allele, for example) can be used to detect the presence of the target sequence of interest. The oligonucleotides can also be used to indicate the amount of the target nucleic acid molecules in comparison to the standard control, based on the intensity of signal imparted by the oligonucleotide species.

Sequence-specific oligonucleotide species hybridization can be used to detect a particular nucleic acid in a mixture or mixed population comprising other species of nucleic acids. Under sufficiently stringent hybridization conditions, the oligonucleotide species (e.g., probes) hybridize specifically only to substantially complementary sequences. The stringency of the hybridization conditions can be relaxed to tolerate varying amounts of sequence mismatch. A number of hybridization formats are known in the art, which include but are not limited to, solution phase, solid phase, or mixed phase hybridization assays. The following documents provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

Hybridization complexes can be detected by techniques known in the art. Nucleic acid probes (e.g., oligonucleotide species) capable of specifically hybridizing to a target nucleic acid (e.g., mRNA or amplified DNA) can be labeled by any suitable method, and the labeled probe used to detect the presence of hybridized nucleic acids. One commonly used method of detection is autoradiography, using probes labeled with 3H, 125I, 35S, 14C, 32P, or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. In some embodiments, probes can be conjugated directly with labels such as fluorophores, chemiluminescent agents or enzymes. The choice of label depends on sensitivity required, ease of conjugation with the probe, stability requirements, and available instrumentation.

“Primer extension” polymorphism detection methods, also referred to herein as “microsequencing” methods, typically are carried out by hybridizing a complementary oligonucleotide species to a nucleic acid carrying the polymorphic site. In these methods, the oligonucleotide typically hybridizes adjacent to the polymorphic site. The term “adjacent” as used in reference to “microsequencing” methods, refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 1, 2, or 3 nucleotides, and the number and/or type of nucleotides that are added to the extension oligonucleotide determine which polymorphic variant or variants are present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. The extension products can be detected in any manner, such as by fluorescence methods (see, e.g., Chen & Kwok, Nucleic Acids Research 25: 347-353 (1997) and Chen et al., Proc. Natl. Acad. Sci. USA 94/20: 10756-10761 (1997)) or by mass spectrometric methods (e.g., MALDI-TOF mass spectrometry) and other methods described herein. Oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods often incorporate an amplification process that precedes the extension step. The amplification process typically amplifies a region from a nucleic acid sample that comprises the polymorphic site. Amplification can be carried out utilizing methods described above, below in the example section or for example using a pair of oligonucleotide species compositions described herein, in a polymerase chain reaction (PCR), in which one oligonucleotide species typically is complementary to a region 3′ of the polymorphism and the other typically is complementary to a region 5′ of the polymorphism. A PCR oligonucleotide species pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR oligonucleotide species pairs may also be used in any commercially available machines that perform PCR, such as any of the GeneAmp® Systems available from Applied Biosystems.

Whole genome sequencing may also be utilized for discriminating alleles of target nucleic acids (e.g., RNA transcripts or DNA), in some embodiments. Examples of whole genome sequencing methods include, but are not limited to, nanopore-based sequencing methods, sequencing by synthesis and sequencing by ligation, as described above.

Data Processing

The term “detection” of one or more cleavage products or cleavage fragments (collectively referred to hereafter as “a cleavage product” or “cleavage products”), as used herein, refers to detecting a product of an endonuclease cleavage reaction by a suitable method. Any suitable detection device and method can be used to detect a cleavage product, as addressed herein. In some embodiments, one or more cleavage fragments may be detected (e.g., two cleavage products may be detected by mass spectrometry; one cleavage product having a detectable label may be detected by detecting a signal emitted by the detectable label).

The term “outcome” as used herein refers to a phenotype indicated by the presence or absence of a cleavage product. Non-limiting examples of outcomes include presence or absence of a fetus, chromosome abnormality, chromosome aneuploidy (e.g., trisomy 21, trisomy 18, trisomy 13) or disease condition. An outcome also can be presence or absence of a cleavage product. Presence or absence of an outcome can be expressed in any suitable form, including, without limitation, ratio, deviation in ratio, frequency, distribution, probability (e.g., odds ratio, p-value), likelihood, percentage, value over a threshold, or risk factor, associated with the presence of a outcome for a subject or sample. An outcome may be provided with one or more of sensitivity, specificity, standard deviation, coefficient of variation (CV) and/or confidence level, or combinations of the foregoing, in certain embodiments.

Presence or absence of an outcome may be determined for all samples tested, and in some embodiments, presence or absence of a outcome is determined in a subset of the samples (e.g., samples from individual pregnant females). In certain embodiments, an outcome is determined for about 60, 65, 70, 75, 76, 77, 78, 79, 80, 81, 82, 83, 84, 85, 86, 87, 88, 89, 90, 91, 92, 93, 94, 95, 96, 97, 98, 99%, or greater than 99%, of samples analyzed in a set. A set of samples can include any suitable number of samples, and in some embodiments, a set has about 10, 15, 20, 25, 30, 35, 40, 45, 50, 55, 60, 65, 70, 75, 80, 90, 100, 200, 300, 400, 500, 600, 700, 800, 900 or 1000 samples, or more than 1000 samples. The set may be considered with respect to samples tested in a particular period of time, and/or at a particular location. The set may be otherwise defined by, for example, gestational age and/or ethnicity. The set may be comprised of a sample which is subdivided into subsamples or replicates all or some of which may be tested. The set may comprise a sample from the same subject collected at two different times. In certain embodiments, an outcome is determined about 60% or more of the time for a given sample analyzed (e.g., about 65, 70, 75, 76, 77, 78, 79, 80, 81, 82, 83, 84, 85, 86, 87, 88, 89, 90, 91, 92, 93, 94, 95, 96, 97, 98, 99%, or more than 99% of the time for a given sample). In certain embodiments, analyzing a higher number of characteristics (e.g., sequence variations) that discriminate alleles can increase the percentage of outcomes determined for the samples (e.g., discriminated in a multiplex analysis). In some embodiments, one or more tissue or fluid samples (e.g., one or more blood samples) are provided by a subject (e.g., pregnant female). In certain embodiments, one or more RNA or DNA samples, or two or more replicate RNA or DNA samples, are isolated from a single tissue or fluid sample, and analyzed by methods described herein.

Presence or absence of an outcome may be identified based on one or more calculated variables, including, but not limited to, ratio, distribution, frequency, sensitivity, specificity, standard deviation, coefficient of variation (CV), a threshold, confidence level, score, probability and/or a combination thereof. In some embodiments, (i) the number of sets selected for a diagnostic method, and/or (ii) the particular nucleotide sequence species of each set selected for a diagnostic method, is determined in part or in full according to one or more of such calculated variables.

In certain embodiments, one or more of ratio, sensitivity, specificity and/or confidence level are expressed as a percentage. In some embodiments, the percentage, independently for each variable, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome determined by an algorithm is not due to chance) in certain embodiments is expressed as a p-value, and sometimes the p-value is about 0.05 or less (e.g., about 0.05, 0.04, 0.03, 0.02 or 0.01, or less than 0.01 (e.g., about 0.001 or less, about 0.0001 or less, about 0.00001 or less, about 0.000001 or less)).

For example, scoring or a score may refer to calculating the probability that a particular outcome is actually present or absent in a subject/sample. The value of a score may be used to determine for example the variation, difference, or ratio of amplified nucleic detectable product that may correspond to the actual outcome. For example, calculating a positive score from detectable products can lead to an identification of an outcome, which is particularly relevant to analysis of single samples.

In certain embodiments, simulated (or simulation) data can aid data processing for example by training an algorithm or testing an algorithm. Simulated data may for instance involve hypothetical various samples of different concentrations of fetal and maternal nucleic acid in serum, plasma and the like. Simulated data may be based on what might be expected from a real population or may be skewed to test an algorithm and/or to assign a correct classification based on a simulated data set. Simulated data also is referred to herein as “virtual” data. Fetal/maternal contributions within a sample can be simulated as a table or array of numbers (for example, as a list of peaks corresponding to the mass signals of cleavage products of a reference biomolecule or amplified nucleic acid sequence), as a mass spectrum, as a pattern of bands on a gel, label intensity, or as a representation of any technique that measures mass distribution. Simulations can be performed in most instances by a computer program. One possible step in using a simulated data set is to evaluate the confidence of the identified results, i.e. how well the selected positives/negatives match the sample and whether there are additional variations. A common approach is to calculate the probability value (p-value) which estimates the probability of a random sample having better score than the selected one. As p-value calculations can be prohibitive in certain circumstances, an empirical model may be assessed, in which it is assumed that at least one sample matches a reference sample (with or without resolved variations). Alternatively other distributions such as Poisson distribution can be used to describe the probability distribution.

In certain embodiments, an algorithm can assign a confidence value to the true positives, true negatives, false positives and false negatives calculated. The assignment of a likelihood of the occurrence of a outcome can also be based on a certain probability model.

Simulated data often is generated in an in silico process. As used herein, the term “in silico” refers to research and experiments performed using a computer. In silico methods include, but are not limited to, molecular modeling studies, karyotyping, genetic calculations, biomolecular docking experiments, and virtual representations of molecular structures and/or processes, such as molecular interactions.

As used herein, a “data processing routine” refers to a process, that can be embodied in software, that determines the biological significance of acquired data (i.e., the ultimate results of an assay). For example, a data processing routine can determine the amount of each nucleotide sequence species based upon the data collected. A data processing routine also may control an instrument and/or a data collection routine based upon results determined. A data processing routine and a data collection routine often are integrated and provide feedback to operate data acquisition by the instrument, and hence provide assay-based judging methods provided herein.

As used herein, software refers to computer readable program instructions that, when executed by a computer, perform computer operations. Typically, software is provided on a program product containing program instructions recorded on a computer readable medium, including, but not limited to, magnetic media including floppy disks, hard disks, and magnetic tape; and optical media including CD-ROM discs, DVD discs, magneto-optical discs, and other such media on which the program instructions can be recorded.

Different methods of predicting abnormality or normality can produce different types of results. For any given prediction, there are four possible types of outcomes: true positive, true negative, false positive, or false negative. The term “true positive” as used herein refers to a subject correctly diagnosed as having a outcome. The term “false positive” as used herein refers to a subject wrongly identified as having a outcome. The term “true negative” as used herein refers to a subject correctly identified as not having a outcome. The term “false negative” as used herein refers to a subject wrongly identified as not having a outcome. Two measures of performance for any given method can be calculated based on the ratios of these occurrences: (i) a sensitivity value, the fraction of predicted positives that are correctly identified as being positives (e.g., the fraction of nucleotide sequence sets correctly identified by level comparison detection/determination as indicative of outcome, relative to all nucleotide sequence sets identified as such, correctly or incorrectly), thereby reflecting the accuracy of the results in detecting the outcome; and (ii) a specificity value, the fraction of predicted negatives correctly identified as being negative (the fraction of nucleotide sequence sets correctly identified by level comparison detection/determination as indicative of chromosomal normality, relative to all nucleotide sequence sets identified as such, correctly or incorrectly), thereby reflecting accuracy of the results in detecting the outcome.

The term “sensitivity” as used herein refers to the number of true positives divided by the number of true positives plus the number of false negatives, where sensitivity (sens) may be within the range of 0 sens 1. Ideally, method embodiments herein have the number of false negatives equaling zero or close to equaling zero, so that no subject is wrongly identified as not having at least one outcome when they indeed have at least one outcome. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0≦spec≦1. Ideally, methods embodiments herein have the number of false positives equaling zero or close to equaling zero, so that no subject wrongly identified as having at least one outcome when they do not have the outcome being assessed. Hence, a method that has sensitivity and specificity equaling one, or 100%, sometimes is selected.

One or more prediction algorithms may be used to determine significance or give meaning to the detection data collected under variable conditions that may be weighed independently of or dependently on each other. The term “variable” as used herein refers to a factor, quantity, or function of an algorithm that has a value or set of values. For example, a variable may be the design of a set of amplified nucleic acid species, the number of sets of amplified nucleic acid species, percent fetal genetic contribution tested, percent maternal genetic contribution tested, type of outcome assayed, type of sex-linked abnormalities assayed, the age of the mother and the like. The term “independent” as used herein refers to not being influenced or not being controlled by another. The term “dependent” as used herein refers to being influenced or controlled by another. For example, a particular chromosome and a trisomy event occurring for the particular chromosome that results in a viable being are variables that are dependent upon each other.

Any suitable type of method or prediction algorithm may be utilized to give significance to the data of the present technology within an acceptable sensitivity and/or specificity. For example, prediction algorithms such as Mann-Whitney U Test, binomial test, log odds ratio, Chi-squared test, z-test, t-test, ANOVA (analysis of variance), regression analysis, neural nets, fuzzy logic, Hidden Markov Models, multiple model state estimation, and the like may be used. One or more methods or prediction algorithms may be determined to give significance to the data having different independent and/or dependent variables of the present technology. And one or more methods or prediction algorithms may be determined not to give significance to the data having different independent and/or dependent variables of the technology described herein. One may design or change parameters of the different variables of methods described herein based on results of one or more prediction algorithms (e.g., number of sets analyzed, types of nucleotide species in each set). For example, applying the Chi-squared test to detection data may suggest that specific ranges of maternal age are correlated to a higher likelihood of having an offspring with a specific outcome, hence the variable of maternal age may be weighed differently verses being weighed the same as other variables.

In certain embodiments, several algorithms may be chosen to be tested. These algorithms then can be trained with raw data. For each new raw data sample, the trained algorithms will assign a classification to that sample (i.e. trisomy or normal). Based on the classifications of the new raw data samples, the trained algorithms' performance may be assessed based on sensitivity and specificity. Finally, an algorithm with the highest sensitivity and/or specificity or combination thereof may be identified.

For a chromosome abnormality, such as aneuploidy for example, chromosome ratio of about 1:1 is expected for a normal, euploid fetus. In some embodiments a ratio of nucleotide sequence species in a set is expected to be about 1.0:1.0, which can indicate the nucleotide sequence species in the set are in different chromosomes present in the same number in the subject. When nucleotide sequence species in a set are on chromosomes present in different numbers in the subject (for example, in trisomy 21) the set ratio which is detected is lower or higher than about 1.0:1.0. Where extracellular nucleic acid is utilized as template nucleic acid, the measured set ratio often is not 1.0:1.0 (euploid) or 1.0:1.5 (e.g., trisomy 21), due to a variety of factors. The expected measured ratio can vary, so long as such variation is substantially reproducible and detectable. For example, a particular set might provide a reproducible measured ratio (for example of peaks in a mass spectrograph) of 1.0:1.2 in a euploid measurement. The aneuploid measurement for such a set might then be, for example, 1.0:1.3. The, for example, 1.3 versus 1.2 measurement is the result of measuring the fetal nucleic acid against a background of maternal nucleic acid, which decreases the signal that would otherwise be provided by a “pure” fetal sample, such as from amniotic fluid or from a fetal cell.

As noted above, algorithms, software, processors and/or machines, for example, can be utilized to (i) process detection data pertaining to cleavage products, and/or (ii) identify the presence or absence of a outcome.

In certain embodiments, provided are methods for identifying the presence or absence of an outcome that comprise: (a) providing a system, wherein the system comprises distinct software modules, and wherein the distinct software modules comprise a signal detection module, a logic processing module, and a data display organization module; (b) detecting signal information indicating the presence or absence of a cleavage product; (c) receiving, by the logic processing module, the signal information; (d) calling the presence or absence of an outcome by the logic processing module; and (e) organizing, by the data display organization model in response to being called by the logic processing module, a data display indicating the presence or absence of the outcome.

Provided also are methods for identifying the presence or absence of an outcome, which comprise providing signal information indicating the presence or absence of a cleavage product; providing a system, wherein the system comprises distinct software modules, and wherein the distinct software modules comprise a signal detection module, a logic processing module, and a data display organization module; receiving, by the logic processing module, the signal information; calling the presence or absence of an outcome by the logic processing module; and, organizing, by the data display organization model in response to being called by the logic processing module, a data display indicating the presence or absence of the outcome.

Provided also are methods for identifying the presence or absence of an outcome, which comprise providing a system, wherein the system comprises distinct software modules, and wherein the distinct software modules comprise a signal detection module, a logic processing module, and a data display organization module; receiving, by the logic processing module, signal information indicating the presence or absence of a cleavage product; calling the presence or absence of an outcome by the logic processing module; and, organizing, by the data display organization model in response to being called by the logic processing module, a data display indicating the presence or absence of the outcome.

By “providing signal information” is meant any manner of providing the information, including, for example, computer communication means from a local, or remote site, human data entry, or any other method of transmitting signal information. The signal information may generated in one location and provided to another location.

By “obtaining” or “receiving” signal information is meant receiving the signal information by computer communication means from a local, or remote site, human data entry, or any other method of receiving signal information. The signal information may be generated in the same location at which it is received, or it may be generated in a different location and transmitted to the receiving location.

By “indicating” or “representing” the amount is meant that the signal information is related to, or correlates with, for example, the amount of cleavage product or presence or absence of cleavage product. The information may be, for example, the calculated data associated with the presence or absence of cleavage product as obtained, for example, after converting raw data obtained by mass spectrometry.

Also provided are computer program products, such as, for example, a computer program products comprising a computer usable medium having a computer readable program code embodied therein, the computer readable program code adapted to be executed to implement a method for identifying the presence or absence of an outcome, which comprises (a) providing a system, wherein the system comprises distinct software modules, and wherein the distinct software modules comprise a signal detection module, a logic processing module, and a data display organization module; (b) detecting signal information indicating the presence or absence of a cleavage product; (c) receiving, by the logic processing module, the signal information; (d) calling the presence or absence of an outcome by the logic processing module; and, organizing, by the data display organization model in response to being called by the logic processing module, a data display indicating the presence or absence of the outcome.

Also provided are computer program products, such as, for example, computer program products comprising a computer usable medium having a computer readable program code embodied therein, the computer readable program code adapted to be executed to implement a method for identifying the presence or absence of an outcome, which comprises providing a system, wherein the system comprises distinct software modules, and wherein the distinct software modules comprise a signal detection module, a logic processing module, and a data display organization module; receiving signal information indicating the presence or absence of a cleavage product; calling the presence or absence of an outcome by the logic processing module; and, organizing, by the data display organization model in response to being called by the logic processing module, a data display indicating the presence or absence of the outcome.

Signal information may be, for example, mass spectrometry data obtained from mass spectrometry of a cleavage product, or of amplified nucleic acid. As the cleavage product may be amplified into a nucleic acid that is detected, the signal information may be detection information, such as mass spectrometry data, obtained from stoichiometrically produced nucleic acid from the cleavage product. The mass spectrometry data may be raw data, such as, for example, a set of numbers, or, for example, a two dimensional display of the mass spectrum. The signal information may be converted or transformed to any form of data that may be provided to, or received by, a computer system. The signal information may also, for example, be converted, or transformed to identification data or information representing an outcome. An outcome may be, for example, a fetal allelic ratio, or a particular chromosome number in fetal cells. Where the chromosome number is greater or less than in euploid cells, or where, for example, the chromosome number for one or more of the chromosomes, for example, 21, 18, or 13, is greater than the number of other chromosomes, the presence of a chromosomal disorder may be identified.

Also provided is a machine for identifying the presence or absence of an outcome wherein the machine comprises a computer system having distinct software modules, and wherein the distinct software modules comprise a signal detection module, a logic processing module, and a data display organization module, wherein the software modules are adapted to be executed to implement a method for identifying the presence or absence of an outcome, which comprises (a) detecting signal information indicating the presence or absence of a cleavage product; (b) receiving, by the logic processing module, the signal information; (c) calling the presence or absence of an outcome by the logic processing module, wherein a ratio of alleles different than a normal ratio is indicative of a chromosomal disorder; and (d) organizing, by the data display organization model in response to being called by the logic processing module, a data display indicating the presence or absence of the outcome. The machine may further comprise a memory module for storing signal information or data indicating the presence or absence of a chromosomal disorder. Also provided are methods for identifying the presence or absence of an outcome, wherein the methods comprise the use of a machine for identifying the presence or absence of an outcome.

Also provided are methods identifying the presence or absence of an outcome that comprises: (a) detecting signal information, wherein the signal information indicates presence or absence of a cleavage product; (b) transforming the signal information into identification data, wherein the identification data represents the presence or absence of the outcome, whereby the presence or absence of the outcome is identified based on the signal information; and (c) displaying the identification data.

Also provided are methods for identifying the presence or absence of an outcome that comprises: (a) providing signal information indicating the presence or absence of a cleavage product; (b) transforming the signal information representing into identification data, wherein the identification data represents the presence or absence of the outcome, whereby the presence or absence of the outcome is identified based on the signal information; and (c) displaying the identification data.

Also provided are methods for identifying the presence or absence of an outcome that comprises: (a) receiving signal information indicating the presence or absence of a cleavage product; (b) transforming the signal information into identification data, wherein the identification data represents the presence or absence of the outcome, whereby the presence or absence of the outcome is identified based on the signal information; and (c) displaying the identification data.

For purposes of these, and similar embodiments, the term “signal information” indicates information readable by any electronic media, including, for example, computers that represent data derived using the present methods. For example, “signal information” can represent the amount of a cleavage product or amplified nucleic acid. Signal information, such as in these examples, that represents physical substances may be transformed into identification data, such as a visual display, that represents other physical substances, such as, for example, a chromosome disorder, or a chromosome number. Identification data may be displayed in any appropriate manner, including, but not limited to, in a computer visual display, by encoding the identification data into computer readable media that may, for example, be transferred to another electronic device (e.g., electronic record), or by creating a hard copy of the display, such as a print out or physical record of information. The information may also be displayed by auditory signal or any other means of information communication. In some embodiments, the signal information may be detection data obtained using methods to detect a cleavage product. Once the signal information is detected, it may be forwarded to the logic-processing module. The logic-processing module may “call” or “identify” the presence or absence of an outcome.

Provided also are methods for transmitting genetic information to a subject, which comprise identifying the presence or absence of an outcome wherein the presence or absence of the outcome has been determined from determining the presence or absence of a cleavage product from a sample from the subject; and transmitting the presence or absence of the outcome to the subject. A method may include transmitting prenatal genetic information to a human pregnant female subject, and the outcome may be presence or absence of a chromosome abnormality or aneuploidy, in certain embodiments.

The term “identifying the presence or absence of an outcome” or “an increased risk of an outcome,” as used herein refers to any method for obtaining such information, including, without limitation, obtaining the information from a laboratory file. A laboratory file can be generated by a laboratory that carried out an assay to determine the presence or absence of an outcome. The laboratory may be in the same location or different location (e.g., in another country) as the personnel identifying the presence or absence of the outcome from the laboratory file. For example, the laboratory file can be generated in one location and transmitted to another location in which the information therein will be transmitted to the subject. The laboratory file may be in tangible form or electronic form (e.g., computer readable form), in certain embodiments.

The term “transmitting the presence or absence of the outcome to the subject” or any other information transmitted as used herein refers to communicating the information to the subject, or family member, guardian or designee thereof, in a suitable medium, including, without limitation, in verbal, document, or file form.

Also provided are methods for providing to a subject a medical prescription based on genetic information, which comprise identifying the presence or absence of an outcome, wherein the presence or absence of the outcome has been determined from the presence or absence of a cleavage product from a sample from the subject; and providing a medical prescription based on the presence or absence of the outcome to the subject.

The term “providing a medical prescription based on prenatal genetic information” refers to communicating the prescription to the subject, or family member, guardian or designee thereof, in a suitable medium, including, without limitation, in verbal, document or file form.

The medical prescription may be for any course of action determined by, for example, a medical professional upon reviewing the prenatal genetic information. For example, the prescription may be for a pregnant female subject to undergo an amniocentesis procedure. Or, in another example, the medical prescription may be for the subject to undergo another genetic test. In yet another example, the medical prescription may be medical advice to not undergo further genetic testing.

Also provided are files, such as, for example, a file comprising the presence or absence of a chromosomal disorder in the fetus of the pregnant female subject, wherein the presence or absence of the outcome has been determined from the presence or absence of a cleavage product in a sample from the subject.

Also provided are files, such as, for example, a file comprising the presence or absence of outcome for a subject, wherein the presence or absence of the outcome has been determined from the presence or absence of a cleavage product in a sample from the subject. The file may be, for example, but not limited to, a computer readable file, a paper file, or a medical record file.

Computer program products include, for example, any electronic storage medium that may be used to provide instructions to a computer, such as, for example, a removable storage device, CD-ROMS, a hard disk installed in hard disk drive, signals, magnetic tape, DVDs, optical disks, flash drives, RAM or floppy disk, and the like.

The systems discussed herein may further comprise general components of computer systems, such as, for example, network servers, laptop systems, desktop systems, handheld systems, personal digital assistants, computing kiosks, and the like. The computer system may comprise one or more input means such as a keyboard, touch screen, mouse, voice recognition or other means to allow the user to enter data into the system. The system may further comprise one or more output means such as a CRT or LCD display screen, speaker, FAX machine, impact printer, inkjet printer, black and white or color laser printer or other means of providing visual, auditory or hardcopy output of information.

The input and output means may be connected to a central processing unit which may comprise among other components, a microprocessor for executing program instructions and memory for storing program code and data. In some embodiments the methods may be implemented as a single user system located in a single geographical site. In other embodiments methods may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by the provider or it may be implemented as an Internet based service where the user accesses a web page to enter and retrieve information.

The various software modules associated with the implementation of the present products and methods can be suitably loaded into the a computer system as desired, or the software code can be stored on a computer-readable medium such as a floppy disk, magnetic tape, or an optical disk, or the like. In an online implementation, a server and web site maintained by an organization can be configured to provide software downloads to remote users. As used herein, “module,” including grammatical variations thereof, means, a self-contained functional unit which is used with a larger system. For example, a software module is a part of a program that performs a particular task. Thus, provided herein is a machine comprising one or more software modules described herein, where the machine can be, but is not limited to, a computer (e.g., server) having a storage device such as floppy disk, magnetic tape, optical disk, random access memory and/or hard disk drive, for example.

The present methods may be implemented using hardware, software or a combination thereof and may be implemented in a computer system or other processing system. An example computer system may include one or more processors. A processor can be connected to a communication bus. The computer system may include a main memory, sometimes random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

In alternative embodiments, secondary memory may include other similar means for allowing computer programs or other instructions to be loaded into a computer system. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to a computer system.

The computer system may also include a communications interface. A communications interface allows software and data to be transferred between the computer system and external devices. Examples of communications interface can include a modem, a network interface (such as an Ethernet card), a communications port, a PCMCIA slot and card, etc. Software and data transferred via communications interface are in the form of signals, which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels. Thus, in one example, a communications interface may be used to receive signal information to be detected by the signal detection module.

In a related aspect, the signal information may be input by a variety of means, including but not limited to, manual input devices or direct data entry devices (DDEs). For example, manual devices may include, keyboards, concept keyboards, touch sensitive screens, light pens, mouse, tracker balls, joysticks, graphic tablets, scanners, digital cameras, video digitizers and voice recognition devices. DDEs may include, for example, bar code readers, magnetic strip codes, smart cards, magnetic ink character recognition, optical character recognition, optical mark recognition, and turnaround documents. In one embodiment, an output from a gene or chip reader my serve as an input signal.

### Examples

The examples set forth below illustrate, and do not limit, the technology.

### Example 1

### General Method for Detecting Nucleic Acids Using Primers Containing Endonuclease Cleavage Substrates

Target nucleic acid sequences can be amplified and/or detected using abasic oligonucleotides species blocked at the 3′ end and an AP endonuclease. Target nucleic acid sequences also can be amplified and/or detected using blocked oligonucleotide species containing other endonuclease cleavage sites (restriction enzymes or nicking enzymes). The 3′ block prevents the oligonucleotides from being used for primer extension or target amplification. The abasic site, or restriction endonuclease recognition site, allows for specific cleavage of the oligonucleotide by an endonuclease. The method can be adapted to make use of thermostable endonucleases, thus allowing the method to be used in conjunction with thermocycling techniques (e.g., PCR, thermocycle sequencing and the like).

The general method comprises; (i) contacting oligonucleotide species with nucleic acid compositions, under hybridizing conditions (ii) cleaving the endonuclease cleavage site, under cleavage conditions, and (iii) extending the functional cleavage site under extension or amplification conditions. In some embodiments a detection step may be included after (iii).

Oligonucleotide species compositions described herein can be used for direct detection of a nucleic acid or to prevent unwanted artifacts caused by inaccurate template priming (e.g., primer dimers and the like). Oligonucleotide species may be designed to have a sequence complementary to a target nucleic acid or a sequence complementary to a sequence near a target nucleic acid. The oligonucleotides include an endonuclease cleavage site at, or near, the center of the primer, and a 3′ blocking agent. The oligonucleotides also may include one or more capture agents and/or features that can be used for detection or identification of (i) a target nucleic acid, or (ii) completion of a particular step in a reaction or completion of the entire reaction. The sequences of the oligonucleotide species can be designed such that an intact oligonucleotide has an annealing temperature (Tm) near the optimal temperature for function of a thermostable polymerase and/or thermostable endonuclease, and the cleaved oligonucleotide species fragments have a lower Tm than intact oligonucleotides. Oligonucleotide species designed in this manner can be readily used in thermocycling-based methods, where the temperature of the extension reactions will cause some or all of the cleaved primer fragments to dissociate from the template. Those primers that do not dissociate, but are in the path of a polymerase extending from an upstream oligonucleotide, may be displaced by strand displacement activity of the advancing polymerase. Oligonucleotides may also be designed such that the portion of the oligonucleotide 5′ to the endonuclease cleavage site has a Tm that allows the portion upstream of the cleavage sit to remain annealed and act as a polymerase priming site for extension or amplification. Additional method specific details are provided in the examples below.

To be useful for unblocking of blocked oligonucleotide species in an extension or amplification reaction, the unblocking reaction should occur at or above the temperature at which unblocked oligonucleotide species are designed to anneal. If they are unblocked at a significantly lower temperature the polymerase could potentially initiate amplification from non-specifically annealed oligonucleotides. Additionally, the endonuclease should leave a free 3′ hydroxyl, when being used to unblock oligonucleotides, so that the oligonucleotides can be extended by a polymerase. Site-specific endonucleases that require the least specificity in the oligonucleotide species 3′ end design allow the most flexibility in the design process.

### Example 2

### Amplifying Target Nucleic Acid Compositions Using Blocked Primers Containing Endonuclease Cleavage Substrates and Thermostable Endonucleases

This method may be performed using a 3′ blocked oligonucleotide with an endonuclease cleavage substrate (an abasic site or a restriction endonuclease site) and a 5′ feature suitable for use as a capture agent or a detectable feature, and one or more unmodified primers (e.g., forward and/or reverse primers), or the method may be performed using two or more 3′ blocked oligonucleotides with an endonuclease cleavage substrate (an abasic site or a restriction endonuclease site) and an optional 5′ feature suitable for use as a capture agent or a detectable feature. For embodiments using two or more 3′ blocked oligonucleotide with an endonuclease cleavage substrate, the Tm of the portion of the oligonucleotide 5′ to the cleavage site is substantially similar to the temperature used for extension or amplification conditions. The portion of the cleaved oligonucleotide 3′ to the cleavage site is designed to have a Tm lower than the temperature used in extension or amplification conditions. For embodiments using only one 3′ blocked oligonucleotide with an endonuclease cleavage substrate, the sequence of the oligonucleotide is designed such that the Tm of both cleaved fragments is below the temperature used for extension or amplification conditions.

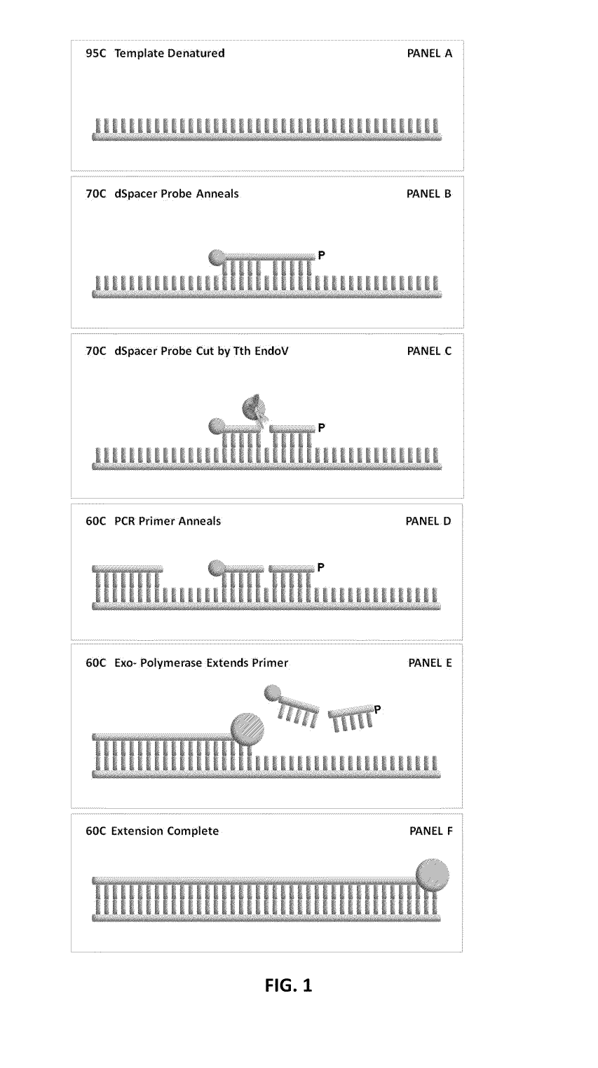

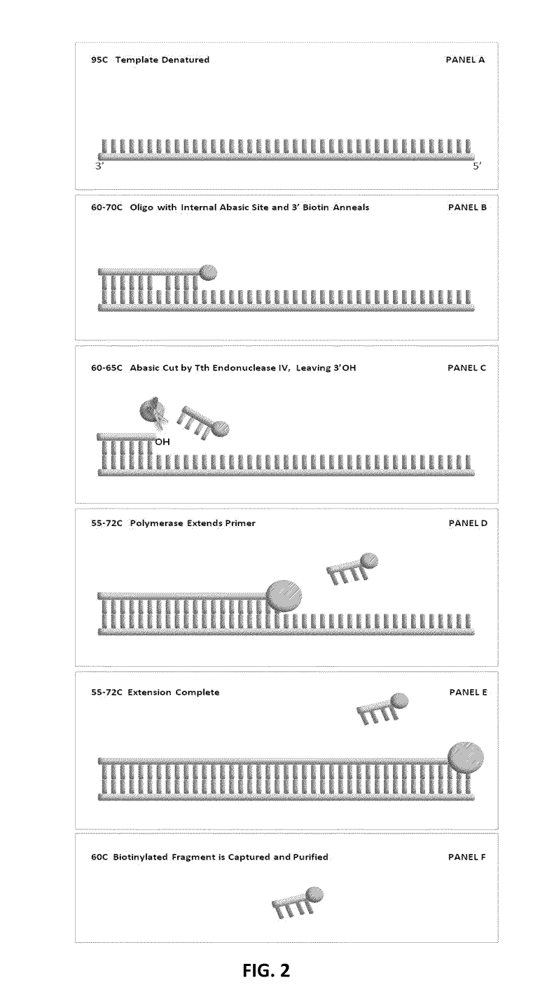

FIG. 1 illustrates a method embodiment using a 3′ blocked abasic oligonucleotide with an AP endonuclease cleavage substrate and a 5′ capture agent. FIG. 2 illustrates a method embodiment using at least two 3′ blocked abasic oligonucleotides with AP endonuclease cleavage sites.

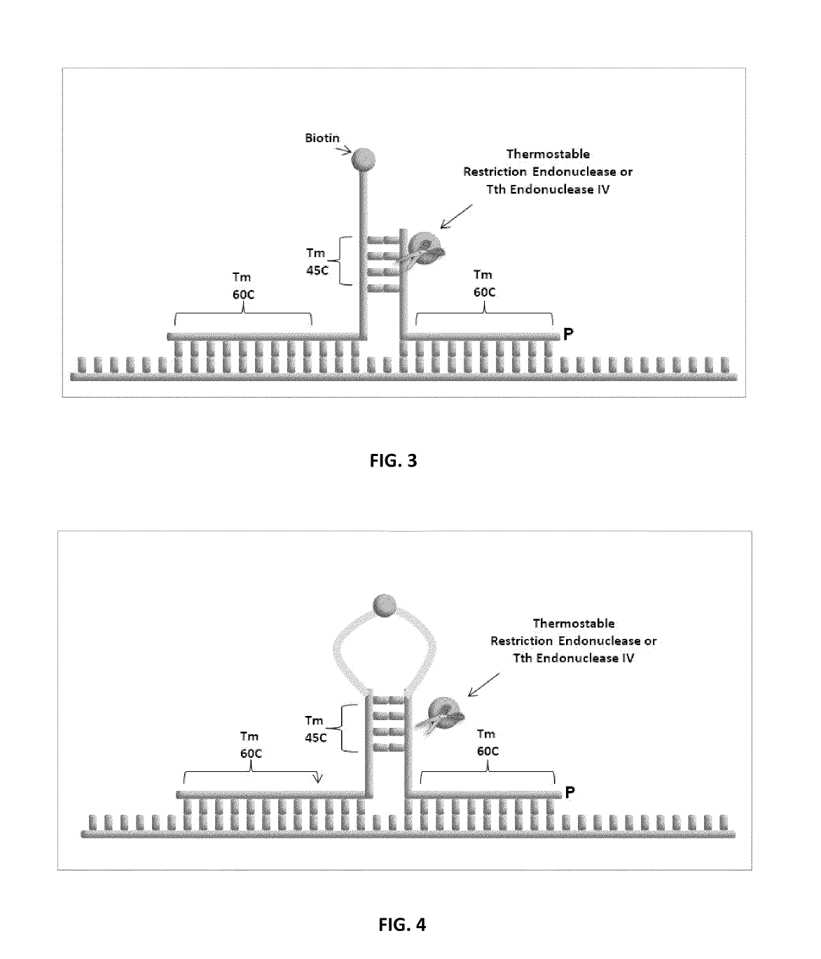

FIG. 3 illustrates a dual oligonucleotide structure for use as a hybridization probe or as a blocked oligonucleotide for extension or amplification methods. This design can be used as an internal hybridization probe or as a blocked primer assay. The two oligonucleotides are complementary to neighboring regions on the target. At the correct Tm (for example, 60 C in this example) they will anneal near each other leaving some small number of bases in between the hybridized oligonucleotides. The 3′end of the upstream oligo is complementary to the 5 end of the downstream oligo and not complementary to any sequence in the template DNA. An endonuclease will recognize the structure and cut it, releasing a biotinylated tag and leaving free 3′ hydroxyls. In certain embodiments, the oligonucleotide also can be used in a fluorescent assay by adding a fluorescent moiety (for example, FAM) to the 3′ end of the upstream oligonucleotide and a quencher to the 5′end of the downstream oligonucleotide.

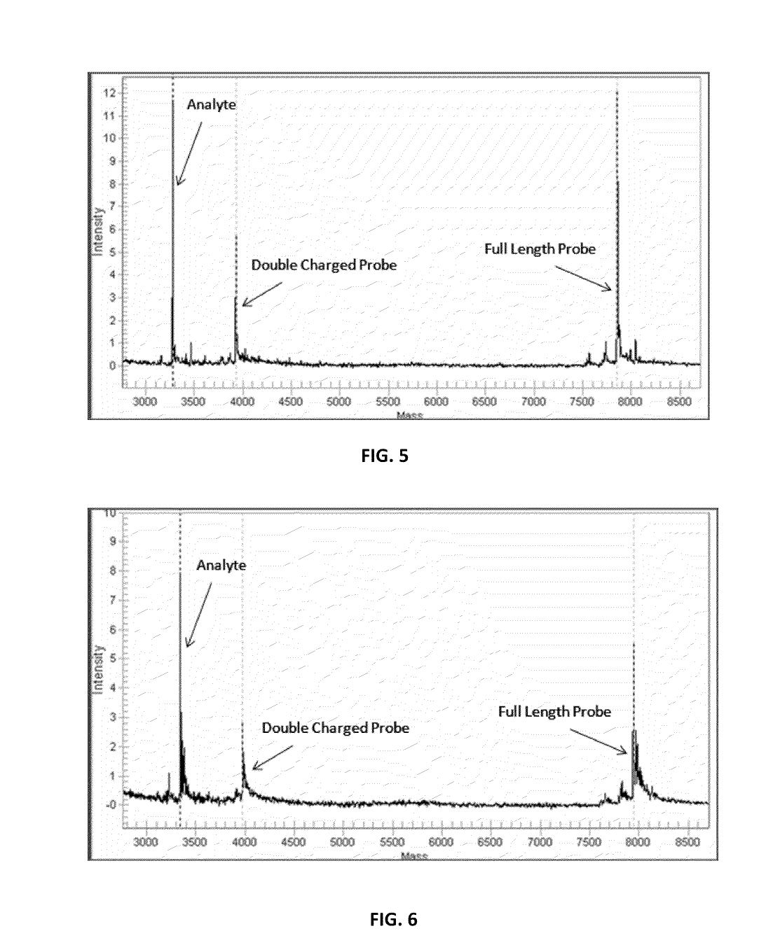

FIG. 4 illustrates an oligonucleotide with internal stem-loop structure that can be used as a hybridization probe or as a blocked oligonucleotide for extension or amplification methods. The oligonucleotide may contain regions that are complementary to neighboring regions on the target. At the correct Tm (for example, 60 C in the example) the regions may anneal near each other leaving some small number of bases in between the hybridized oligonucleotides. The internal region of the oligonucleotide forms a stem-loop structure that is not complementary to any sequence in the template DNA. The Tm of the internal structure is too low for it to form a stem-loop structure, unless the two sides are brought together by the annealing of the 5′ and 3′ ends to the template (e.g., the reverse of a molecular beacon). The oligonucleotide also can be used in a fluorescent assay by adding a fluorescent moiety (for example, FAM) to the 3′ end of the upstream oligonucleotide or internally in the loop structure and a quencher to the 5′end of the downstream oligonucleotide, in some embodiments. The endonuclease cleavage site can be designed to cut the stem-loop in a manner that includes or excludes a portion of a two part detectable feature (e.g., a two part fluorophore system for example).

The dual oligonucleotide structure and the stem-loop structure oligonucleotides are designed using the same strategies described above. The protocols for using the oligonucleotides species describe FIGS. 2-4 are substantially similar to that described for the embodiment illustrated in FIG. 1.

Illustrated in FIG. 1 is a method that makes use of Tth Endonuclease IV to cleave an internal hybridization probe in a PCR assay using a 5′˜3′ exonuclease-minus DNA polymerase. In this particular embodiment the assay uses an unmodified forward primer, an unmodified reverse primer and a biotinylated internal hybridization probe with an internal abasic site. The 3′ end of the probe is blocked to prevent extension. When annealed, the endonuclease cleaves at the abasic site. Cleaved probe fragments have free 3′ hydroxyls but are not extended by the polymerase, because the fragments have Tm's below the annealing temperatures of the intact oligonucleotide species composition. FIGS. 3-7 illustrate the results of MALDI mass spectrometry detection of oligonucleotides extended from cleaved blocked primers using extension and amplification methods described herein.

The DNA polymerase used in this version of the assay does not contain the 5′˜3′exonuclease activity that is needed for a TaqMan assay. The probe is not cleaved by the DNA polymerase.

Examples of DNA polymerases lacking the 5′to 3′exonuclease activity include Deep VentR™ (exo˜) DNA Polymerase, Phire Hot Start DNA Polymerase, Phusion DNA Polymerase and the Stoffel fragment for Taq DNA Polymerase.
Deep VentR™ (exo˜) DNA Polymerase (New England Biolabs, Ipswich Mass.) has been genetically engineered to eliminate the 3′ to 5′ proofreading exonuclease activity associated with Deep Vent DNA Polymerase. Deep VentR DNA Polymerase is purified from a strain of E. coli that carries the Deep VentR DNA Polymerase gene from Pyrococcus species GB˜D.
Phire Hot Start DNA Polymerase (Finnzymes, Inc., Woburn Mass.) is constructed by fusing a DNA polymerase (orange) and a small double stranded DNA binding protein (yellow). This technology increases the processivity of the polymerase and improves its overall performance. It contains a 3′ to 5′exonuclease activity but not a 5′ to 3′exonuclease activity.
Phusion DNA Polymerase (Finnzymes, Inc., Woburn Mass.) is a chimeric protein that fuses a novel Pyrococcus like DNA polymerase with a processivity enhancing domain. It contains a 3′ to 5′ exonuclease activity but not a 5′ to 3′ exonuclease activity.
Stoffel fragment (Applied Biosystems, Foster City Calif.) is a truncated version of Taq DNA polymerase protein and is missing the 5′ to 3′ exonuclease domain.

The following oligonucleotide sequences were used to demonstrate the use of Tth Endonuclease IV to cleave an internal hybridization probe in an amplification reaction. All the following examples were generated using 20 μL reactions containing: 1× Thermopol buffer (20 mM Tris˜HCl, 10 mM (NH₄)₂SO₄, 10 mM KCl, 2 mM MgSO₄ and 0.1% Triton X˜100), 25 μM ZnCl₂, 125 μM dATP, 125 μM dCTP, 125 μM dGTP, 125 μM dTTP, 2.5 units Tth Endonuclease IV, and 5 ng human genomic DNA. The DNA polymerase was added at 1 unit per 20 μL reaction.

Oligonucleotide sequences which are annotated with /5BioTEG/ contain a biotin attached to the 5′end of the oligo by an extended 15˜atom spacer arm. Oligo sequences which are annotated with /dSpacer/ or /idSp/ contain a 1′,2′˜Dideoxyribose or dSpacer. The 1′,2′˜Dideoxyribose modification is used to introduce a stable abasic site within an oligonucleotide. It is this modification that is cleaved by the Tth Endonuclease IV. It is more stable than a standard abasic site and also may be called abasic furan. Oligonucleotide sequences which are annotated with /3Phos/ contain a phosphate at the 3′ position. In this example use of a 3′phosphate (instead of a 3′hydroxyl) prevents DNA polymerase from extending the hybridization oligo. Other moieties may be substituted at the 3′terminus to prevent DNA polymerase from extending the oligo. Such moieties may include but are not limited to 3′ Amino Modifiers, 3′ Biotin, 3′Biotin TEG, 3′ Cholestery1˜TEG, 3′ Digoxigenin, 3′ Thiol, 3′ Inverted dT or 3′ C3 Spacer.

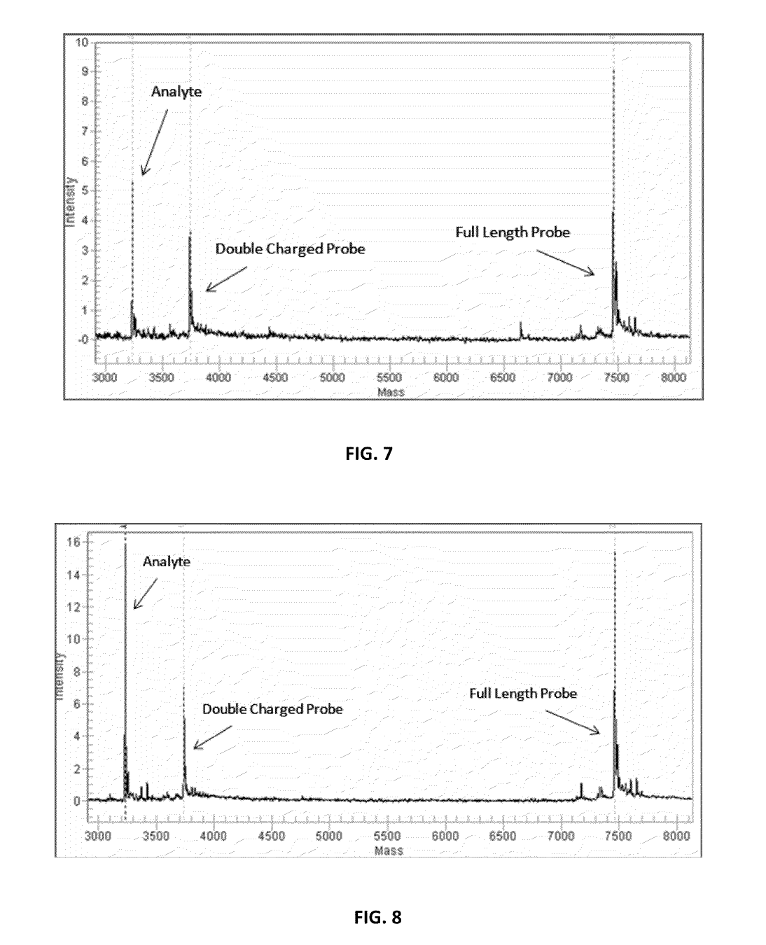

Illustrated in FIG. 5 is a Tth endonuclease assay using an oligonucleotide having an internal hybridization probe. The assay was performed with Deep Vent (exo˜) DNA polymerase and a 3-step thermocycling protocol of 95 C for 3 min, followed by 99 cycles of 95 C for 20 sec and 60 C for 2 minutes. Reactions were subsequently purified by capture of the 5′biotin moiety with Streptavidin-coated paramagnetic beads. The oligonucleotide sequences were designed against the Homo sapiens SRY gene for sex determining region Y, isolate ADT3 (GenBank AM884751.1). The oligonucleotide sequences are as follows;

| | |
|---|---|
| Forward Primer: | |
| SRY.CTA.Tth.f3 | GAATGCGAAACTCAGAGATCA |
| Reverse Primer: | |
| SRY.CTA.Tth.r3 | CCTGTAATTTCTGTGCCTCCT |
| Internal Probe: | |
| SRY.CTA.Tth.p3 | /5BioTEG/ACTGAAGCC/dSpacer/ |
| | AAAAATGGCCATTC/3 Phos/ |
| Analyte on MALDI: | /5BioTEG/ACTGAAGCC |

The intact probe has a mass of 7855.3 daltons, and the cleaved tag or analyte has a mass of 3277.4 daltons.

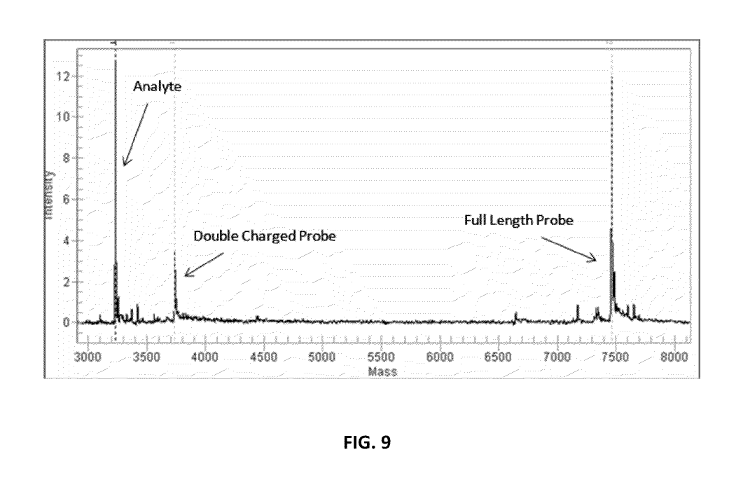

Illustrated in FIG. 6 is a Tth endonuclease assay using an oligonucleotide having an internal hybridization probe. The assay was performed with Deep Vent (exo˜) DNA Polymerase and a 2-step thermocycling protocol of 95 for 3 min, followed by 99 cycles of 95 C for 20 sec and 60 C for 2 min. Reactions were subsequently purified by capture of the 5′biotin moiety with Streptavidin-coated paramagnetic beads. The oligonucleotide sequences were designed against the Homo sapiens SRY gene for sex determining region Y, isolate ADT3 (GenBank AM884751.1). The oligonucleotide sequences are as follows;

| | |
|---|---|
| Forward Primer: | |
| SRY.CTA.Tth.f4 | AAATG CTTACTGAAGCCGAAA |
| Reverse Primer: | |
| SRY.CTA.Tth.r4 | CG GGTATTTCTCTCTGTG CAT |
| Internal Probe: | |
| SRY.CTA.Tth.p4 | /5 BioTEG/CAG GAG GCA/dSpacer/ |
| | AGAAATTACAGGCC/3 Phos/ |
| Analyte on MALDI: | /5BioTEG/CAGGAGGCA |

The intact probe has a mass of 7945.4 daltons, and the cleaved tag or analyte has a mass of 3342.4 daltons.

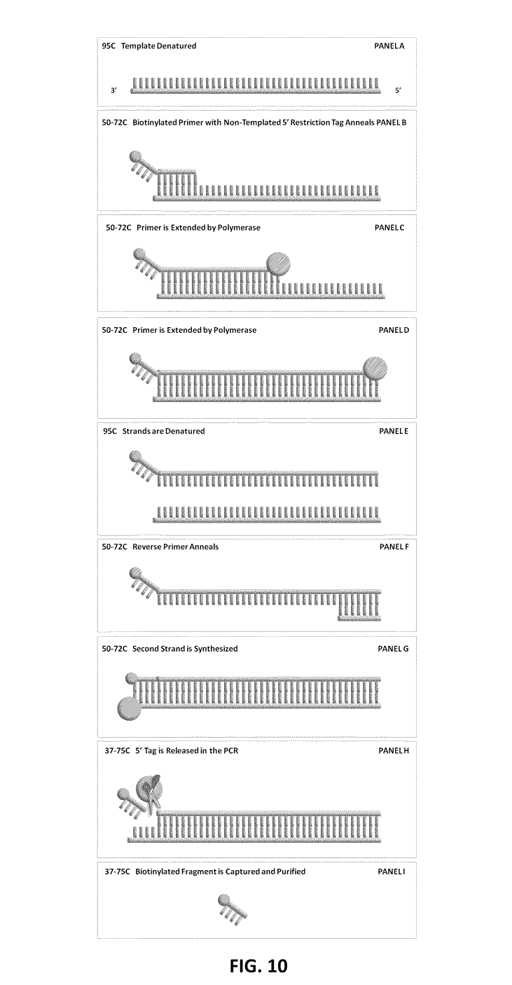

Illustrated in FIG. 7 is a Tth endonuclease assay using an oligonucleotide having an internal hybridization probe. The assay was performed with Stoffel fragment of Taq DNA Polymerase and a 3 step thermocycling protocol of 95 for 3 min, followed by 99 cycles of 95 C for 20 sec and 60 C for 2 minutes. Reactions were subsequently purified by capture of the 5′biotin moiety with Streptavidin-coated paramagnetic beads. The oligonucleotide sequences were designed against the Homo sapiens SRY gene for sex determining region Y, isolate ADT3 (GenBank AM884751.1). The oligonucleotide sequences are as follows

| | |
|---|---|
| Forward Primer: | |
| SRY.CTA.Tth.f2 | GTCCAG CTGTGCAAGAGAATA |
| Reverse Primer: | |
| SRY.CTA.Tth.r2 | TACAG CTTTCAGTGCAAAGGA |
| Internal Probe: | |
| SRY.CTA.Tth.p2 | /5BioTEG/CGC TCT CCG/dSpacer/ |
| | AGAAGCTCT TCCT/3Phos/ |
| Analyte on MALDI: | /5BioTEG/CGCTCTCCG |

The intact probe has a mass of 7452.0 daltons, and the cleaved tag or analyte has a mass of 3220.4 daltons.

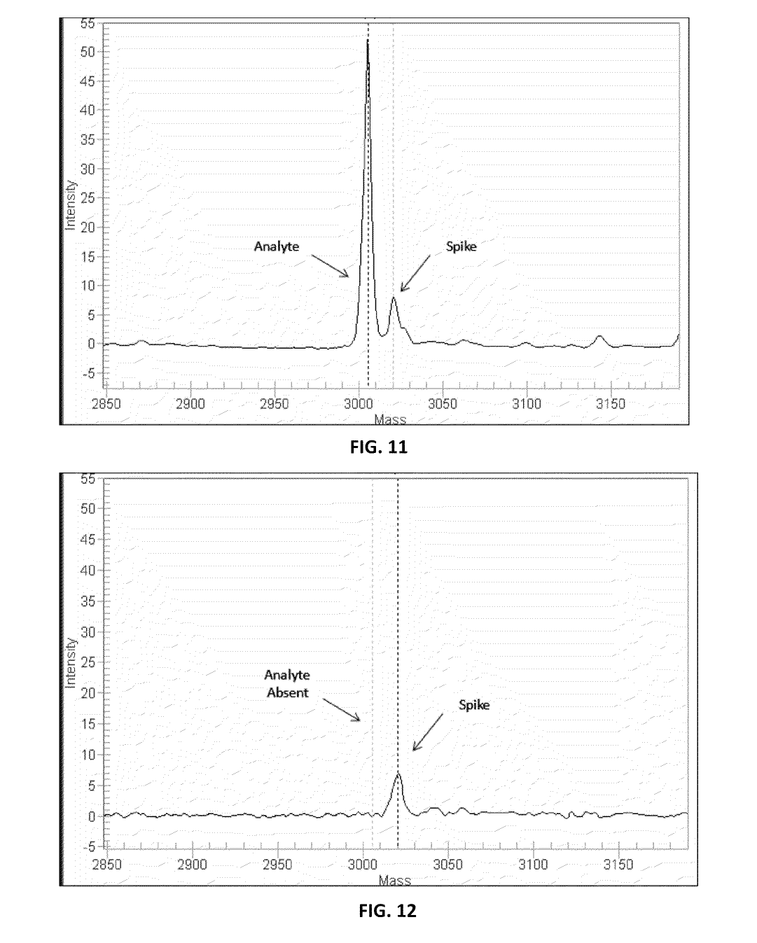

Illustrated in FIG. 8 is a Tth endonuclease assay using an oligonucleotide having an internal hybridization probe. The assay was performed with Phusion Hot Start DNA Polymerase and a 2˜step thermocycling protocol of 95 for 3 min, followed by 99 cycles of 95 C for 20 sec and 60 C for 2 min. Reactions were subsequently purified by capture of the 5′biotin moiety with Streptavidin-coated paramagnetic beads. The oligonucleotide sequences were designed against the Homo sapiens SRY gene for sex determining region Y, isolate ADT3 (GenBank AM884751.1). The oligonucleotide sequences are as follows;

| | |
|---|---|
| Forward Primer: | |
| SRY.CTA.Tth.f2 | GTCCAG CTGTGCAAGAGAATA |
| Reverse Primer: | |
| SRY.CTA.Tth.r2 | TACAG CTTTCAGTGCAAAGGA |
| Internal Probe: | |
| SRY.CTA.Tth.p2 | /5BioTEG/CGCTCTCCG/dSpacer/ |
| | AGAAGCTCTTCCT/3Phos/ |
| Analyte on MALDI: | /5BioTEG/CGCTCTCCG |

The intact probe has a mass of 7452.0 daltons, and the cleaved tag or analyte has a mass of 3220.4 daltons.

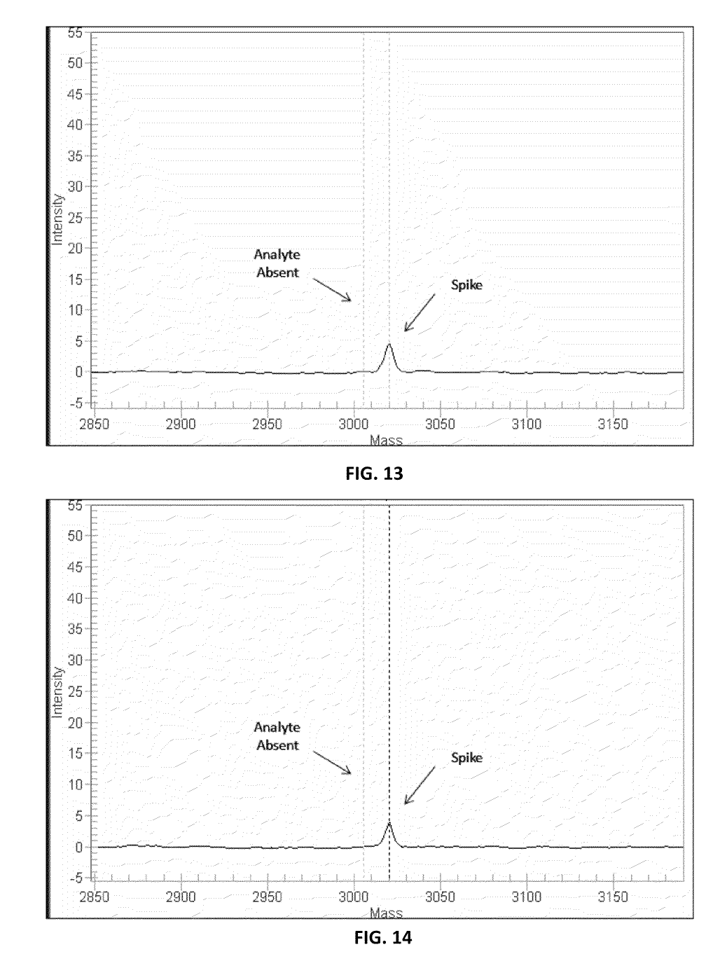

Illustrated in FIG. 9 is a Tth endonuclease assay using an oligonucleotide having an internal hybridization probe. The assay was performed with Phire DNA Polymerase and a 2˜step thermocycling protocol of 95 for 3 min, followed by 99 cycles of 95 C for 20 sec and 60 C for 2 min. Reactions were subsequently purified by capture of the 5′biotin moiety with Streptavidin-coated paramagnetic beads. The oligonucleotide sequences were designed against the Homo sapiens SRY gene for sex determining region Y, isolate ADT3 (GenBank AM884751.1). The oligonucleotide sequences are as follows;

| | |
|---|---|
| Forward Primer: | |
| SRY.CTA.Tth.f2 | GTCCAG CTGTGCAAGAGAATA |
| Reverse Primer: | |
| SRY.CTA.Tth.r2 | TACAG CTTTCAGTGCAAAGGA |
| Internal Probe: | |
| SRY.CTA.Tth.p2 | /5BioTEG/CGCTCTCCG/dSpacer/ |
| | AGAAGCTCTTCCT/3Phos/ |
| Analyte on MALDI: | /5BioTEG/CGCTCTCCG |

The intact probe has a mass of 7452.0 daltons, and the cleaved tag or analyte has a mass of 3220.4 daltons.

FIGS. 1-9 are exemplary of embodiments using oligonucleotides with abasic sites that form AP endonuclease cleavage sites. The skilled artisan will appreciate that restriction enzymes are also endonucleases and that certain restriction enzymes are also thermostable. Therefore the examples above can also include modifications that substitute restriction endonuclease or nicking endonuclease cleavage substrates in place of abasic AP endonuclease substrates, and thermostable restriction enzymes or nicking enzymes for thermostable AP endonucleases.

### Example 3

### Amplifying Target Nucleic Acid Compositions Using Oligonucleotides Containing a Thermostable Restriction Endonuclease and a 5′ Capture and/or Detection Feature; Effect of Heat on Restriction Endonucleases

Restriction endonucleases vary with respect to their ability to maintain activity in a reaction over an extended period of time. For many molecular biology applications, it is convenient to have a method by which restriction endonucleases can be inactivated. For example, if a cleaved fragment is subsequently ligated into a plasmid during a cloning experiment, it is convenient to inactivate the restriction enzyme so that it does not interfere with subsequent manipulations (e.g., cutting possible restriction sequences in the plasmid or in the ligated fragment).

For most molecular biology applications the ability to inactivate the enzymatic activity of a restriction enzyme is important. Most restriction endonucleases are described in their ability to be “heat inactivated.” One such common method of inactivating restriction endonucleases is through denaturation of the protein by heating. The majority of restriction endonucleases that have an optimal incubation temperature of 37° C. can be inactivated by incubation at 65° C. for 20 minutes. Many other enzymes can be inactivated by incubation at 80° C. for 20 minutes. Some restriction endonucleases are not easily inactivated by heat. Therefore, understanding the thermal tolerance, or heat tolerance half-life, of a particular restriction endonuclease is important for the design of oligonucleotide species compositions and thermocycling profiles.

Table 1, “Examples of Heat Tolerance of Restriction Endonucleases”, provides a few examples of restriction endonucleases that can be heat inactivated by incubation at 65° C. for 20 minutes, by incubation at 80° C. for 20 minutes, or that cannot be heat inactivated. If the enzyme can be heat inactivated the time and temperature to accomplish inactivation are listed. This information was compiled from data listed on the New England Biolabs website (World Wide Web URL neb.com). A more comprehensive listing of thermostable restriction endonucleases is provided in Example 9.

TABLE 1

| | Heat | Inactivation | Inactivation |
|---|---|---|---|
| Enzyme | Inactivation | Temperature | Time |
| BamHI | No | ~~ | ~~ |
| BstUI | No | ~~ | ~~ |
| EcoRI | Yes | 65° C. | 20 min |
| EcoRI~HF ™ | Yes | 65° C. | 20 min |
| EcoRV | Yes | 80° C. | 20 min |
| EcoRV~HF ™ | Yes | 65° C. | 20 min |
| HaeII | Yes | 80° C. | 20 min |
| HaeIII | Yes | 80° C. | 20 min |
| HindIII | Yes | 65° C. | 20 min |
| Pvu II | No | ~~ | ~~ |
| PvuII~HF ™ | Yes | 80° C. | 20 min |
| XmaI | Yes | 65° C. | 20 min |

The ability of enzymes to tolerate extended time at high temperature differs between different enzymes as listed in Table 1. Once cloned, restriction endonucleases may be further engineered in vitro to specifically alter their properties such as heat inactivation or tolerance.

Some modified restriction endonucleases maintain the same recognition specificity as their native enzyme. However, certain properties have been altered, including heat tolerance. In order to distinguish these examples of engineered enzymes from the New England Biolabs website they are as listed as “High Fidelity (H F)” restriction enzymes and are designated with the letters—H F™ in Table 1. For example, the Pvu II native enzyme cannot be heat inactivated while the engineered Pvu II-H F™ enzyme is easily heat inactivated by incubation at 80° C. for 20 minutes. While most molecular biology methods will typically prefer to avoid the use of heat tolerant enzymes and prefer enzymes that can be heat inactivated, it is heat tolerance that is exploited in the assays presented herein.

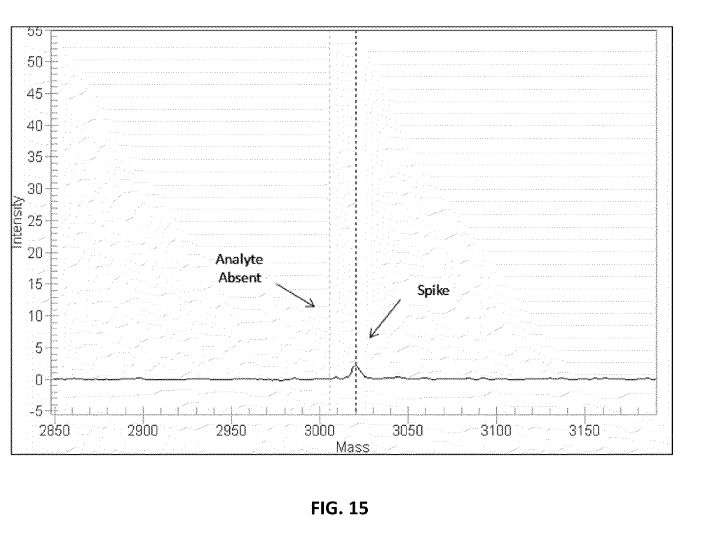

Illustrated in FIG. 10 is a method using an oligonucleotide species composition having a 5′ capture agent and/or detectable feature, and a thermostable restriction endonuclease cleavage substrate sequence. The method uses forward and reverse priming oligonucleotides. One of the oligonucleotides has a 5′biotin. It also has a restriction endonuclease recognition site that contains a sequence that does not occur in the target DNA between the region defined by the forward and reverse priming oligonucleotides. When the second strand is synthesized during the PCR, the restriction endonuclease site and any additional sequence in the oligonucleotide will be copied. Extension from both oligonucleotides forms a double stranded restriction site. The restriction endonuclease will cut the double stranded template, releasing a biotinylated tag. Single stranded unannealed primer will not be cut. The restriction endonuclease digest can be performed either during PCR with an enzyme that cuts at a temperature in the range of about 50 C to about 75 C, or post PCR with an enzyme that cuts at a temperature in the range of about 25 C to about 37 C.

Restriction endonucleases which cleave and leave blunt ends are preferred, because certain DNA polymerases are less likely to modify blunt ends after restriction endonuclease cleavage and thus less likely to alter the expected mass of the analyte. Restriction endonucleases which leave sticky ends (3′overhangs or 5′ overhangs) can be used but potential secondary modifications such as 3 ′ “chew-back” by the 3′˜5′ exonunclease activity of certain DNA polymerases or fill-in of 3′ ends by certain DNA polymerases should be monitored.

In addition to a restriction endonuclease, a thermostable “nicking enzyme” could be used to release the biotinylated tag. A nicking enzyme cuts only one of the two strands of double stranded DNA. The method can also be used in a fluorescent assay by adding a fluorescent moiety (for example, FAM) to the 5′ end of the oligonucleotide containing the upstream restriction site and a quencher to the 3′end of the oligonucleotide. In come embodiments, the fluorescent signal can be doubled by labeling both the forward and reverse oligonucleotides.

Non-limiting examples of thermostable restriction endonuclease useful for the methods described herein, are presented below. Many other restriction endonucleases are available. Additionally cloned sequences of restriction endonucleases can be altered in vitro so that the expressed proteins have altered phenotypes such as increased heat tolerance. Additionally a few restriction enzymes from thermophilic bacteria (for example, Tfil gene from Thermus filiformis from New England Biolabs) are available or may become available in future. The DNA polymerase used in examples presented below does not contain the 5′˜3′exonuclease activity that is needed for a TaqMan assay. The tag or analyte is not cleaved by the DNA polymerase.

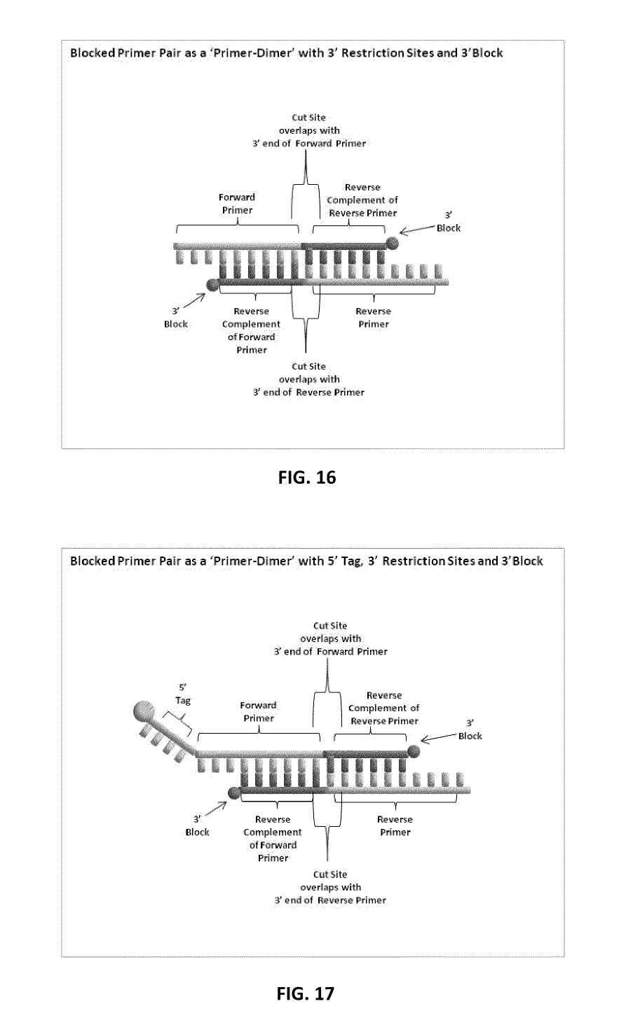

FIGS. 11-15 present examples of the specificity of using Pvu II restriction endonuclease to cleave a 5′ tag or analyte. Pvu II cuts double stranded DNA at the recognition sequence CAGCTG. The reaction is specific because the analyte is not produced if either the Pvu II restriction endonuclease or the DNA are left out of the PCR reaction. The specificity of the reaction is further demonstrated in that it must be thermocycled before incubation at 37 C, to yield the expected analyte.

FIG. 11 illustrates a reaction positive for cleavage of a biotinylated 5′ tag by the Pvu II restriction endonuclease. The sample was amplified in a 3˜step thermocycling protocol of 95 for 3 min, followed by 35 cycles of 95 C for 15 sec, 60 C for 15 sec and 72 C for 30 sec., followed by 37C for one hour. In this example all components were added to produce a positive reaction as indicated by the presence of the analyte peak.

FIG. 12 illustrates a negative reaction (e.g., negative control) for cleavage of a biotinylated 5′ tag by the Pvu II restriction endonuclease. The sample was amplified in a 3-step thermocycling protocol of 95 for 3 min, followed by 35 cycles of 95 C for 15 sec, 60 C for 15 sec and 72 C for 30 sec., followed by 37 C for sec., followed by 37 C for one hour. In this example all components except the Pvu II restriction endonuclease and the genomic DNA were added. Note that the analyte is absent indicating a negative reaction.

FIG. 13 illustrates a negative reaction (e.g., negative control) for cleavage of a biotinylated 5′ tag by the Pvu II restriction endonuclease. The sample was amplified in a 3˜step thermocycling protocol of 95 for 3 min, followed by 35 cycles of 95 C for 15 sec, 60 C for 15 sec and 72 C for 30 sec., followed by 37 C for one hour. In this example all components except the Pvu II restriction endonuclease were added. Note that the analyte is absent indicating a negative reaction.

FIG. 14 illustrates a negative reaction (e.g., negative control) for cleavage of a biotinylated 5′ tag by the Pvu II restriction endonuclease. The sample was amplified in a 3˜step thermocycling protocol of 95 for 3 min, followed by 35 cycles of 95 C for 15 sec, 60 C for 15 sec and 72 C for 30 sec., followed by 37 C for one hour. In this example all components except the genomic DNA were added. Note that the analyte is absent indicating a negative reaction.

FIG. 15 illustrates a negative reaction (e.g., negative control) for cleavage of a biotinylated 5′ tag by the Pvu II restriction endonuclease. In this example all PCR components were added. The reaction was incubated at 37 C for one hour without prior thermocycling. Note that the analyte is absent in the absence of thermocycling, indicating a negative reaction.

The experiments presented in FIGS. 11-15 were performed in a 25 μL reaction with the following (final concentrations): of 1× Taq buffer (50 mM Tris˜HCl, 5 mM (NH4)2SO4, 10 mM KCl, and 4 mM MgCl), 100 μM dATP, 100 μM dCTP, 100 μM dGTP, 100 μM dTTP, 300 nM forward primer, 300 nM reverse primer, 3 nM spike, 2.5 units Roche Fast Start DNA polymerase, 5 units of PvuII restriction endonuclease and 5 ng human genomic DNA. The oligonucleotide sequences used in the examples presented in FIGS. 11-15 were designed against the Homo sapiens SRY gene for sex determining region Y, isolate ADT3 (GenBank AM884751.1). The oligonucleotide sequences are as follows;

| | |
|---|---|
| Forward Primer: | |
| SRY.f1. Pvu II | /5 BioTEG/AAAAACAGCTG CGATCAGAG |
| | GCG CAAGATG |
| Reverse Primer: | |
| SRY.r1.f | G CTGATCTCTGAGTTTCG CATTCTG |
| Analyte on MALDI: | /5BioTEG/AAAAACAG |
| Spike: | |
| SRY1.Spike1L | /5BioTEG/AATCAAAAC |

The intact probe has a mass of 9876.7 daltons, the cleaved tag or analyte has a mass of 3005.2 daltons and the spike has a mass of 3020.3 daltons. Oligonucleotide sequences which are annotated with /5BioTEG/ contain a biotin attached to the 5′end of the oligo by an extended 15˜atom spacer arm. The sample was amplified in a 3˜step thermocycling protocol of 95 for 3 min, followed by 35 cycles of 95 C for 15 sec, 60 C for 15 sec and 72 C for 30 sec., followed by 37 C for one hour. Reactions were subsequently purified by capture of the 5′biotin moiety with Streptavidin-coated paramagnetic beads.

An internal standard or spike is added to the PCR master mix. The spike is 15 daltons higher than the analyte or cleavage product in a positive PCR. The internal standard normalizes for differences in pipetting of PCR reactions, loss through post-PCR handling (e.g., purification with Streptavidin-coated paramagnetic beads and spotting onto MALDI chips), and differences in instrument performance. The peak area response ratio can be calculated for the analyte and the corresponding spike (Bruenner B A, T˜T Yip, T W Hutchens. 1996. Quantitative analysis of oligonucleotides by matrix-assisted laser desorption/ionization of mass spectrometry. Rapid Communications in Mass Spectrometry. 10:1797˜1802).

### Example 4

### Oligonucleotide Species Compositions for Amplifying Target Nucleic Acid Compositions Comprising a Pair of 3′ Blocked Oligonucleotides Having One or More Thermostable Endonuclease Cleavage Substrates and an Optional 5′ Capture and/or Detection Feature

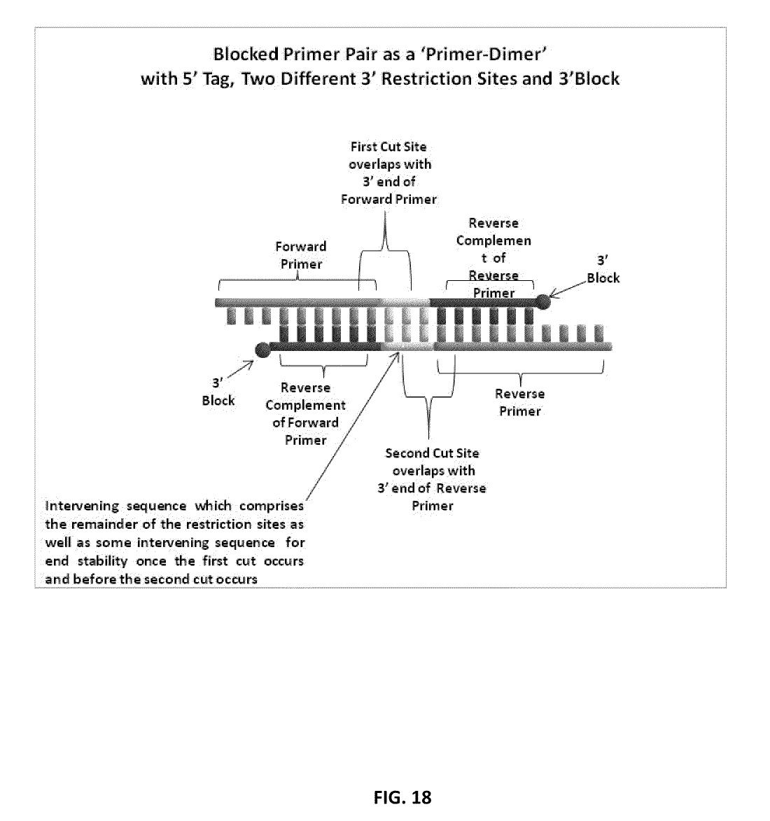

The oligonucleotide compositions described herein also can be designed to function as pairs of oligonucleotides that contain one or more endonuclease cleavage sites, where the cleavage sites can be for the same or different endonucleases. In some embodiments, the forward and reverse oligonucleotides may be unblocked by different endonucleases types (e.g., a restriction endonuclease and an AP endonuclease). The oligonucleotides compositions can also contain 3′ blocks, and optional 5′ capture agents or detectable moieties, as illustrated in FIGS. 16-21B. In embodiments using restriction endonuclease cleavage sites, the restriction endonuclease cleavage site may overlap the 3′ end of the oligonucleotide species compositions as illustrated in FIGS. 16-17. In some embodiments intervening sequences, which can contain a portion of the endonuclease cleavage site, can be included to allow additional spacing for enzymes to bind and for end stability after the first cleavage occurs, but before the second cleavage occurs, as illustrated in FIGS. 18 and 20.

FIG. 16 illustrates a blocked oligonucleotide pair (e.g., primer dimer), with a 3′ block and a restriction endonuclease cleavage site. FIG. 17 illustrates a blocked oligonucleotide pair, with a 5′ tag (e.g., capture agent or detectable moiety), a 3′ block and a restriction site and a restriction site. The embodiments illustrated in FIGS. 16 and 17 comprise forward and reverse oligonucleotide species (e.g., labeled as forward and reverse primers in FIGS. 16 and 17) concatenated with part or all of the reverse complements of the forward and reverse oligonucleotide species and 3′ blocks make a structure similar to a primer dimer. The forward and reverse oligonucleotide species are cleaved by one restriction endonuclease and one cleavage event.

The sequence that is cleaved from the 3′ end of each oligonucleotide species has a lower Tm than the intact oligonucleotides. The temperature at which the oligonucleotide species are used in the subsequent amplification assay is higher than the temperature at which the cleaved 3′ ends will anneal. Thus the cleaved fragments will not interfere with the amplification reactions.

FIG. 18 is exemplary of embodiments comprising a pair of 3′ blocked oligonucleotides with two restriction endonuclease cleavage sites where part of each restriction endonuclease cleavage site is contained in the intervening sequences. In the embodiment illustrated in FIG. 18 the forward and reverse oligonucleotide species (e.g., labeled as forward and reverse primers in FIG. 18) are cut by two different restriction endonucleases that recognize two different cleavage sequences. Intervening sequences contained in the oligonucleotide species compositions completes the remainder of each restriction cleavage site. Intervening sequences may be added to the oligonucleotide species composition to provide additional end stability after the first cut occurs and before the second cut occurs. The sequence that is cleaved from the 3′ end of each oligonucleotide has a lower Tm than the intact oligonucleotides. The temperature at which the oligonucleotide species are used in the subsequent amplification assay is higher than the temperature at which the cleaved 3′ ends will anneal. Thus the cleaved fragments will not interfere with the amplification reactions.

Illustrated in FIGS. 19 and 20 are embodiments substantially similar to those described in FIGS. 17 and 18, with the difference being the formation of two abasic AP endonuclease cleavage sites, instead of a restriction endonuclease site. In the embodiments presented in FIGS. 19 and 20, the forward and reverse oligonucleotides species can be concatenated with part or all of the reverse complements of the forward and reverse oligonucleotide species, and 3′ blocks make a structure similar to a primer dimer. The embodiments in FIGS. 19 and 20 differ by the addition of intervening sequences added to the oligonucleotide species of FIG. 20. The intervening sequences are added to substantially perform the same function as described for embodiments in FIG. 18.

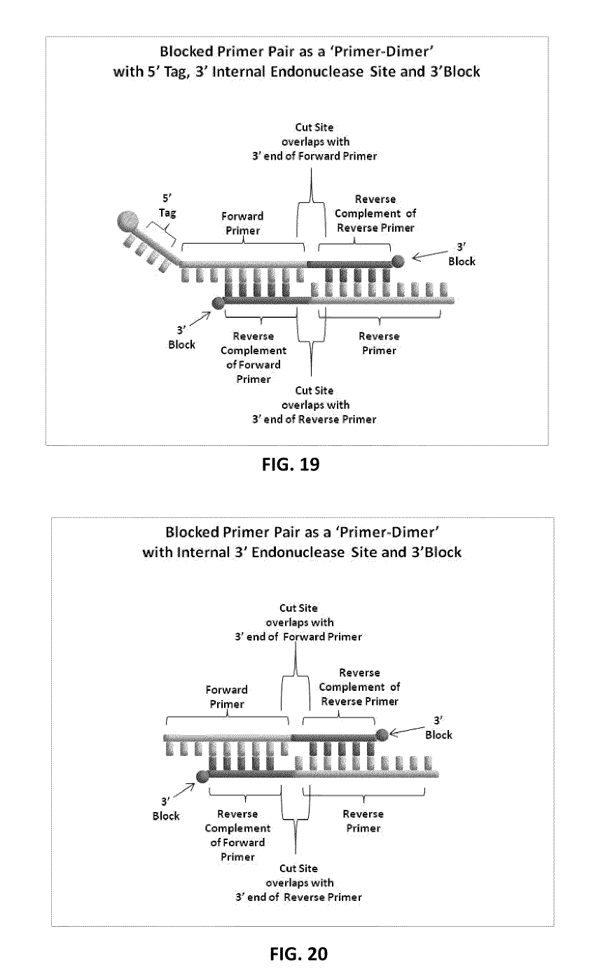

The portion of the sequence that is cleaved from the 3′ end of each oligonucleotide has a lower Tm than the intact oligonucleotide. The temperature at which the oligonucleotides are used in the subsequent amplification assay is higher than the temperature at which the cleaved 3′ ends will anneal. Thus the cleaved fragments will not interfere with the amplification reactions. FIG. 21 is a schematic illustration of the blocked oligonucleotide species compositions being unblocked, by a thermostable AP endonuclease (e.g., Tth IV endonuclease), and generating oligonucleotides useful for extension or amplification methods. Illustrated in FIG. 21 is a non-limiting temperature range in which thermostable endonucleases can function, under cleavage conditions.

In some embodiments a plurality of pairs of oligonucleotide species compositions, each pair containing the same restriction endonuclease site, may be used simultaneously (e.g., in a single tube, or in multiplexed reactions in a single reaction vessel or bound to a solid support, for example). In some embodiments a plurality of pairs of oligonucleotide species compositions, each pair containing a different restriction endonuclease site, may be used simultaneously, or in multiplexed reactions.

### Example 5

### Oligonucleotide Species Compositions for Amplifying Target Nucleic Acid Compositions Comprising Two or More Pairs of 3′ Blocked Oligonucleotides Having One or More Thermostable Endonuclease Cleavage Substrates and Optional 5′ Capture and/or Detection Features

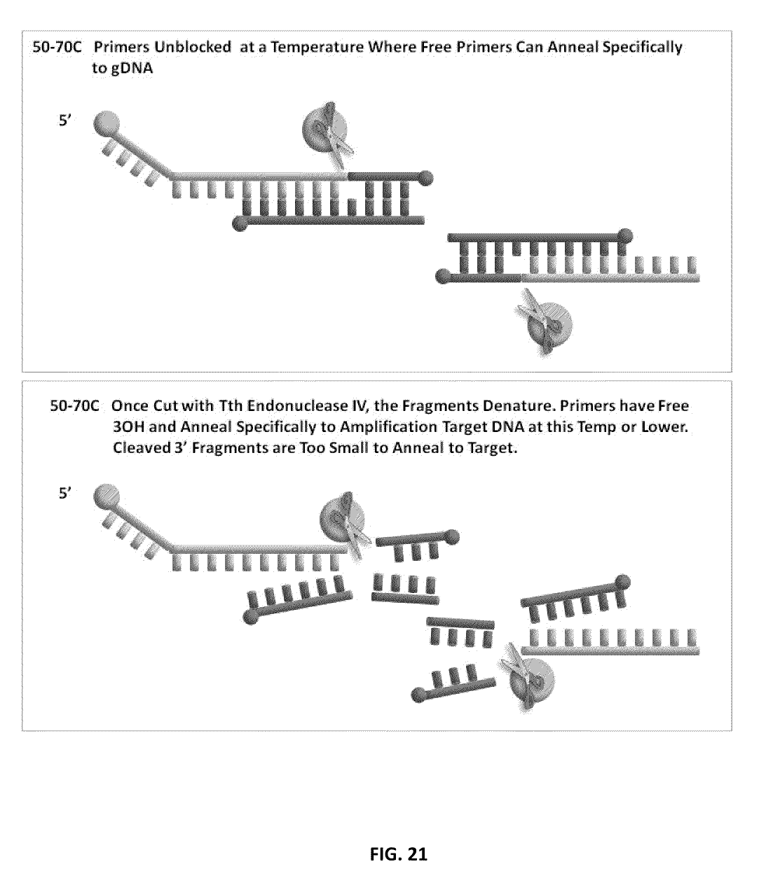

The oligonucleotide compositions described herein also can be designed to function as two or more pairs of oligonucleotides that contain one or more endonuclease cleavage sites, where the cleavage sites can be for the same or different endonucleases. The oligonucleotide compositions can also contain 3′ blocks, and optional 5′ capture agents or detectable moieties, as illustrated in FIGS. 22-26B. In embodiments using restriction endonuclease cleavage sites, the restriction endonuclease cleavage site can overlap the 5′ end of the oligonucleotide species composition (e.g., the portion of the oligonucleotide that serves as the polymerase extension primer), as illustrated in FIGS. 22, 23, 26A and 26B. In some embodiments the 3′ end of an oligonucleotide may contain one half of the restriction enzyme cleavage site. In some embodiments additional sequences, which can contain a portion of the endonuclease cleavage site, can be included at the 3′ end of the oligonucleotide species to allow additional spacing for enzymes to bind and/or for additional thermostability of the cleavage site, as illustrated in FIGS. 22-25. In some embodiments from about 3 to about 20 extra nucleotides can be added to increase binding efficiency and/or thermostability of the cleavage site.

The two or more pairs of nucleotide species compositions can also be referred to as “oligonucleotide species duplexes” or “primer duplexes”. In some embodiments a plurality of oligonucleotide species duplexes, each duplex containing the same restriction endonuclease site, may be used simultaneously (e.g., in a single tube, or in multiplexed reactions in a single reaction vessel or bound to a solid support, for example). In some embodiments a plurality of oligonucleotide species duplexes, each duplex containing a different restriction endonuclease site, may be used simultaneously, or in multiplexed reactions.

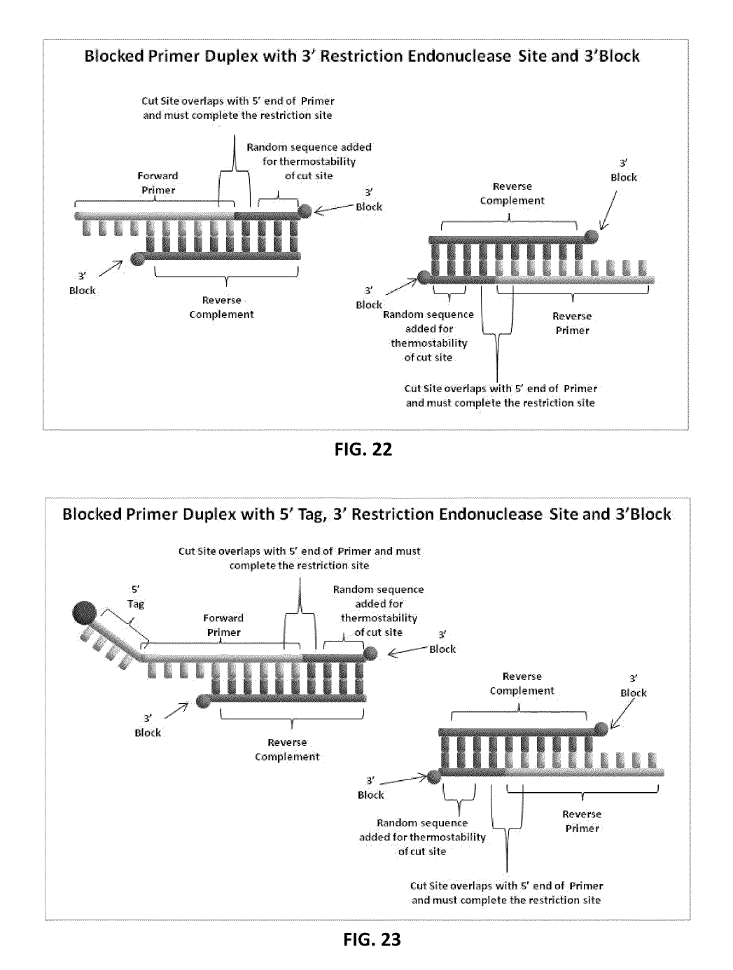

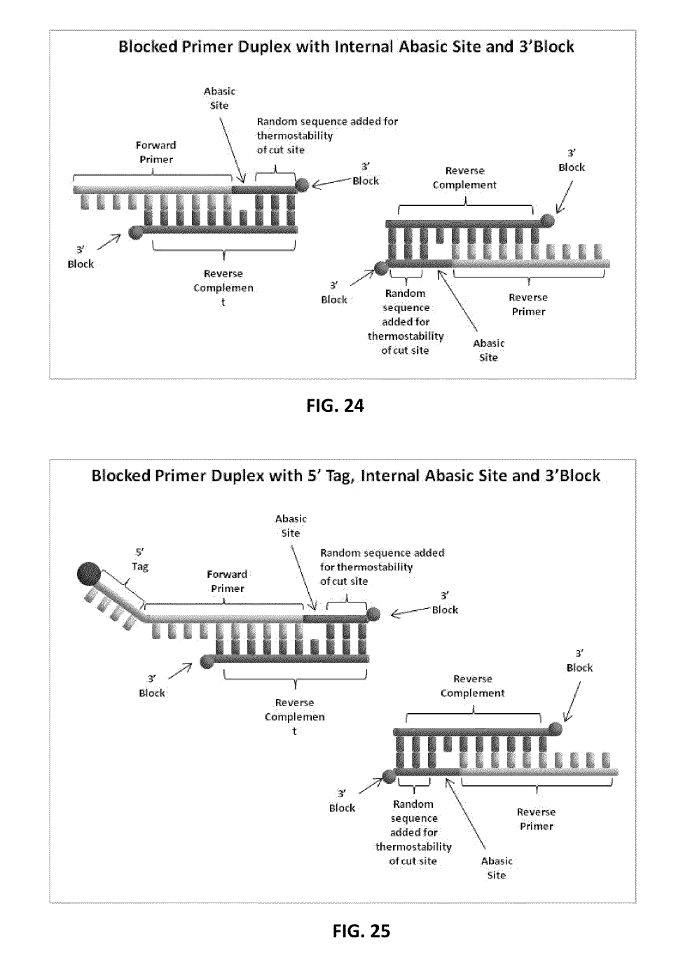

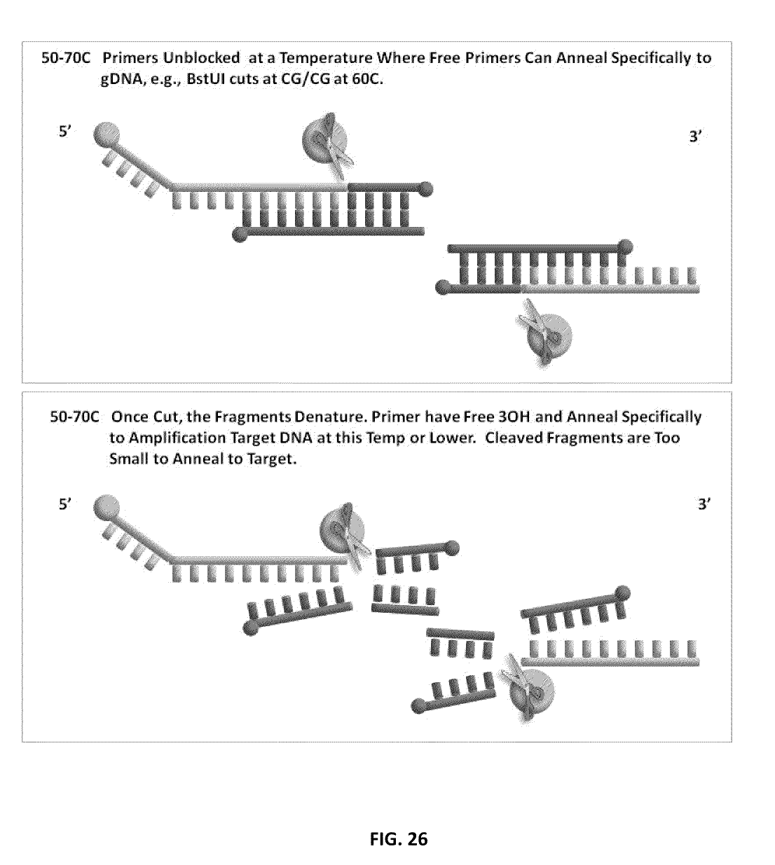

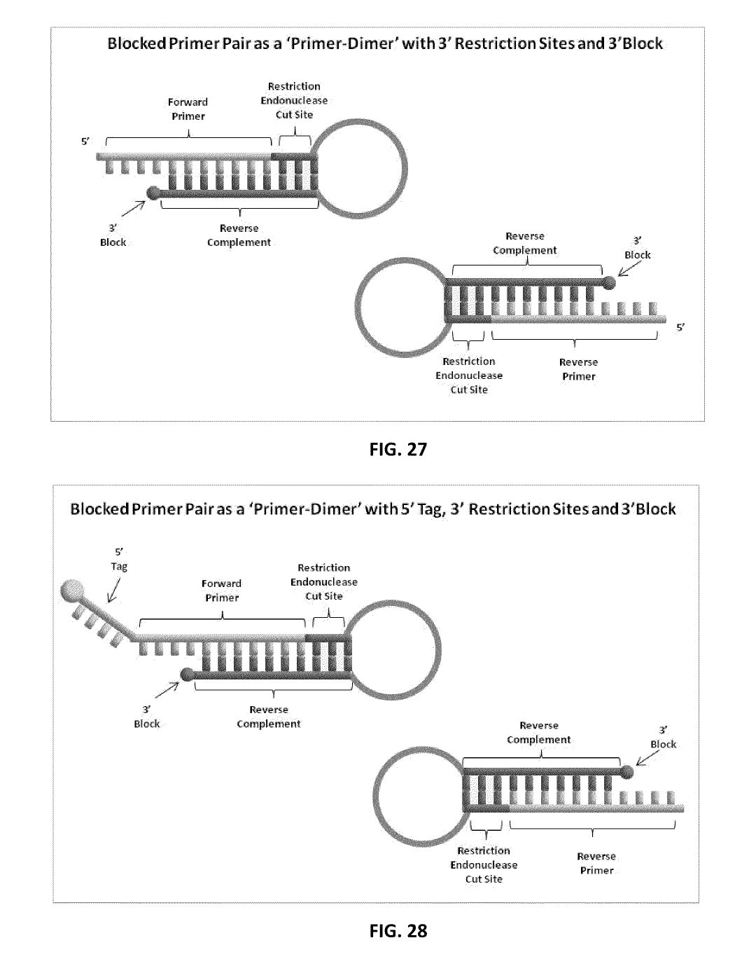

FIGS. 22 and 23 illustrate 3′ blocked oligonucleotide species duplex compositions having one or more thermostable restriction endonuclease cleavage sites. FIG. 23 also illustrates an embodiment having an optional 5′ tag (e.g., capture agent and/or detectable moiety). FIGS. 24 and 25 illustrate 3′ blocked oligonucleotide species duplex compositions having one or more thermostable AP endonuclease cleavage sites. FIG. 25 also illustrates an embodiment having an optional 5′ tag (e.g., capture agent and/or detectable moiety). FIG. 26 is a schematic illustration of the blocked oligonucleotide species compositions being unblocked and generating oligonucleotides useful for extension or amplification methods. The specific, non-limiting example illustrated in FIG. 26 shows cleavage by the restriction endonuclease BstUI, however the oligonucleotide species composition can be designed with any suitable thermostable endonuclease cleavage site.

In the embodiments described in this example and illustrated in FIGS. 22-26B, four independent oligonucleotide species comprise an oligonucleotide species composition duplex. In embodiments using a restriction endonuclease cleavage site, the sequence of the forward and reverse oligonucleotide species (e.g., labeled as forward and reverse primers in FIGS. 22-23) each ends on a partial restriction site, with the rest of the restriction site contained in sequence added (e.g., 3 to 20 bases, for example) to the 3′ end for enzyme binding and cleavage site thermostability. The forward and reverse oligonucleotide species each have a corresponding reverse complement oligonucleotide species that spans the restriction site and will anneal at a temperature in which the restriction endonuclease is active. In FIGS. 24 and 25, the abasic site occurs 3′ to the last nucleotide of the portion of the oligonucleotide species to be used in subsequent extension or amplification reactions.

In some embodiments using restriction endonuclease cleavage sites, the forward and reverse oligonucleotide species may be unblocked by the same restriction endonuclease. In some embodiments, the forward and reverse oligonucleotide species may be unblocked by different restriction endonucleases. In some embodiments, the forward and reverse oligonucleotides may be unblocked by different endonucleases types (e.g., a restriction endonuclease and an AP endonuclease). The sequence that is cleaved from the 3′ end of each oligonucleotide species has a lower Tm than the intact oligonucleotide species. The temperature at which the oligonucleotides are used in the subsequent amplification assay is higher than the temperature at which the cleaved 3′ end or cleaved reverse complement will anneal. Thus the cleaved fragments will not interfere with the amplification reactions.

### Example 6

### Oligonucleotide Species Compositions, for Amplifying Target Nucleic Acid Compositions Comprising a Pair of 3′ Blocked J-Hook Oligonucleotide Species or a Pair of 3′ Blocked Linear Oligonucleotide Species Having Complementary 3′ Ends, One or More Thermostable Endonuclease Cleavage Substrates and an Optional 5′ Capture and/or Detection Feature

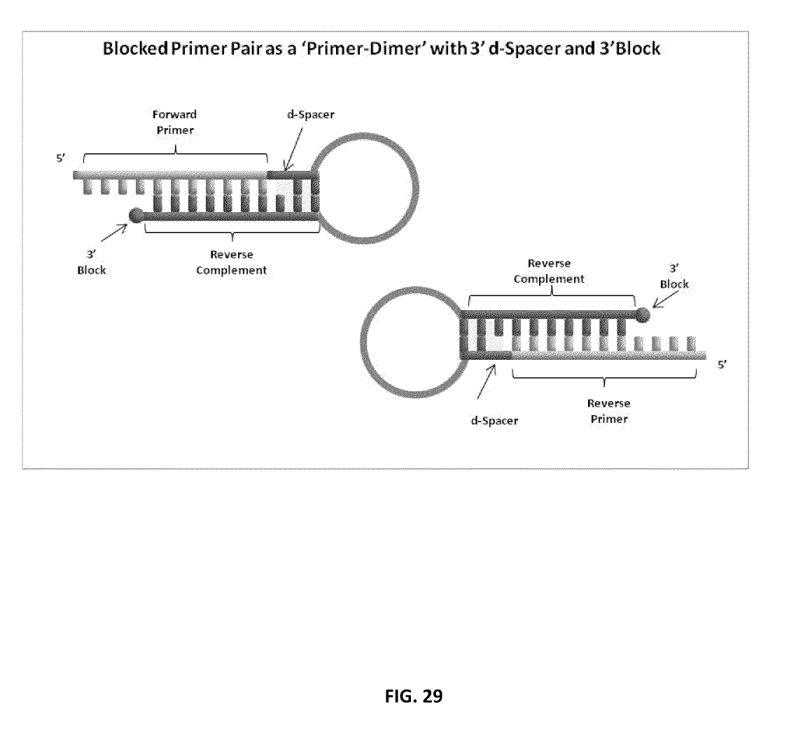

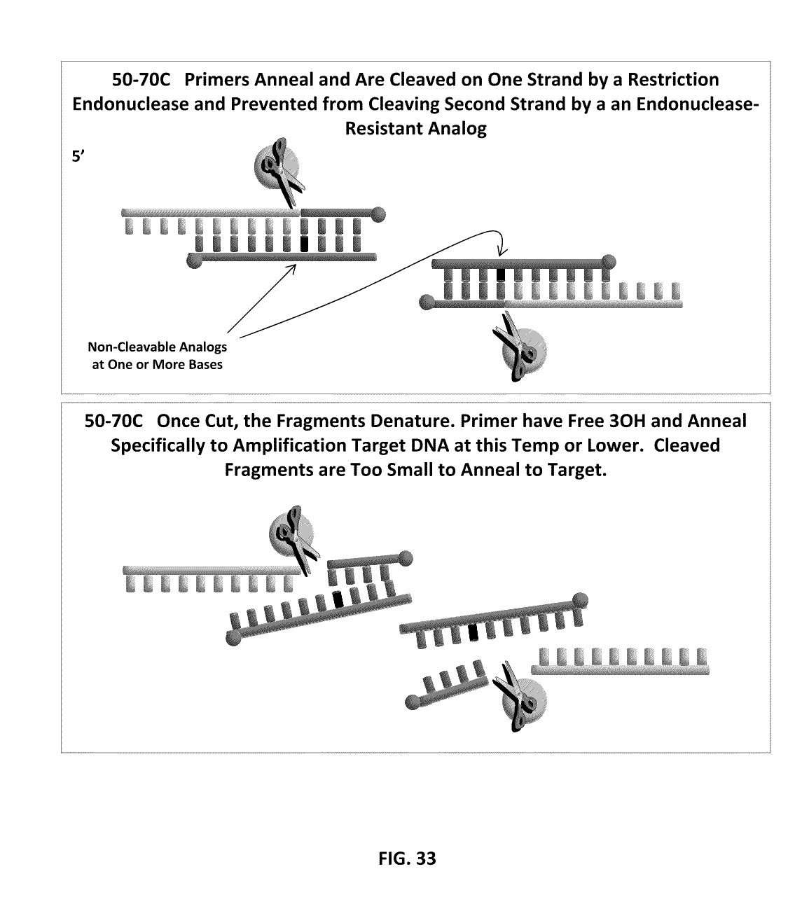

The oligonucleotide compositions described herein can be designed to function as pairs of J-hook oligonucleotide species (illustrated in FIGS. 27-30A) or pairs of 3′ blocked linear oligonucleotide species having complementary 3′ ends (illustrated in FIG. 32), that contain one or more endonuclease cleavage sites, where the cleavage sites can be for the same or different endonucleases. The oligonucleotide compositions can also contain 3′ blocks, and optional 5′ capture agents or detectable moieties, as illustrated in FIGS. 27-30A and FIG. 33. In J-hook oligonucleotide species composition embodiments, an optional internal spacer (illustrated in FIG. 31) may be incorporated to allow additional flexibility to allow the self-complementary portions of the oligonucleotides to anneal.

Design principles substantially similar to those described in the embodiments above (e.g., use of one or more similar or different endonuclease sites, use of different types of endonuclease sites, use of capture agents and/or detectable moieties, use of blocked 3′ ends, Tm considerations for intact and cleaved oligonucleotides, endonuclease cleavage sites overlapping the 5′ or 3′ portion of the oligonucleotide species compositions, cleavage site positioned to allow the individual portions of two part detectable moieties to remain on the same or different cleavage fragments and the like), also may be used in the design of J-hook, and linear oligonucleotide species with complementary 3′ ends, composition pairs.

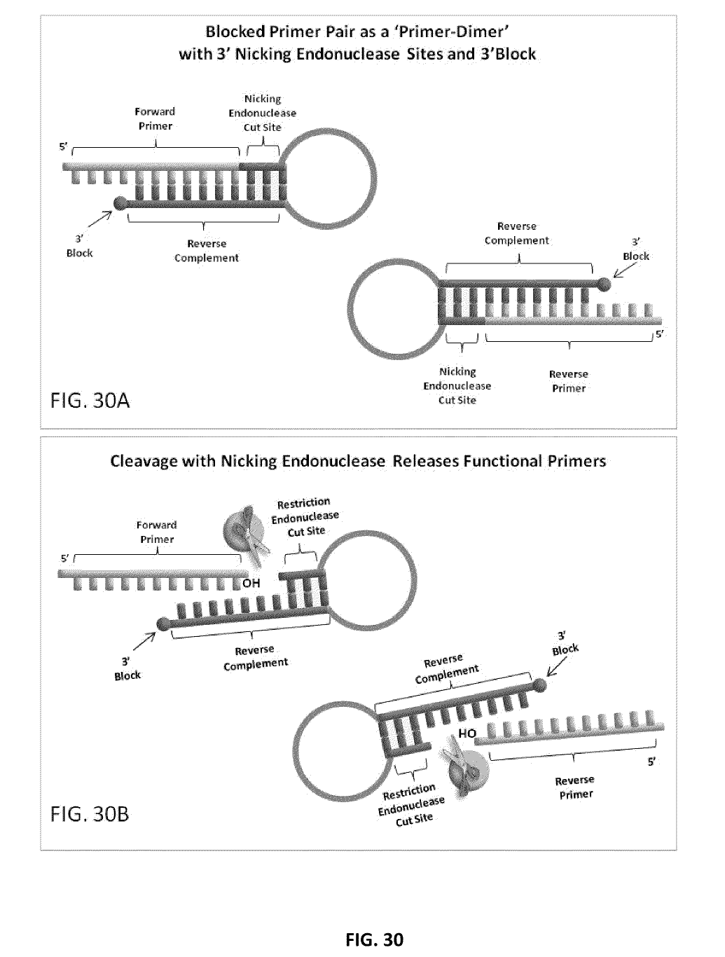

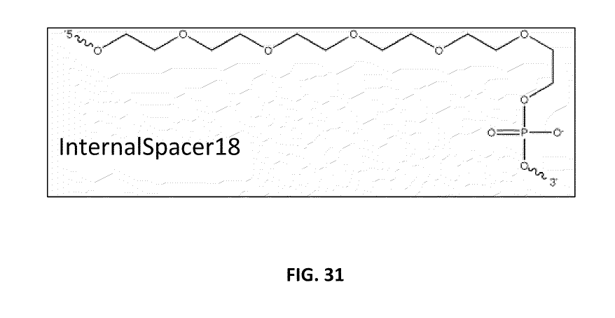

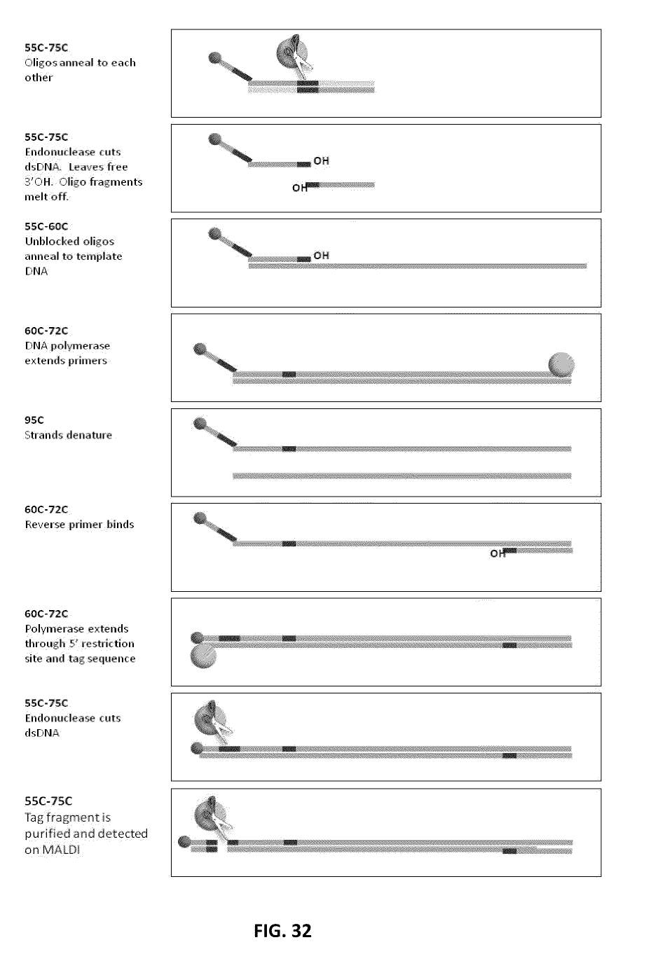

FIGS. 27 and 28 illustrate 3′ blocked J-hook oligonucleotide species pairs with restriction endonuclease cleavage sites, and an optional 5′ capture agent and/or detectable moiety (FIG. 28). In some embodiments, the restriction endonuclease cleavage site is for a thermostable restriction endonuclease. FIG. 29 illustrates 3′ blocked J-hook oligonucleotide species pairs with thermostable AP endonuclease cleavage sites. FIG. 30 illustrates 3′ blocked J-hook oligonucleotide species pairs with nicking endonuclease cleavage sites. 5′ capture agents and/or detectable moieties also may be optionally included, in some embodiments.

In the embodiments illustrated in FIGS. 27-30, the oligonucleotides that make up the pairs of J-hook oligonucleotide species compositions, fold over in a J-Hook with self-complementarity at their 3′ ends. Cleavage with an endonuclease (e.g., restriction endonuclease, thermostable restriction endonuclease, AP endonuclease, thermostable AP endonuclease and the like) makes a cut (e.g., double stranded for restriction endonucleases, single stranded for AP endonucleases) in the oligonucleotide, releasing the block and leaving a free 3′OH on the portion of the oligonucleotide that can be extended by a DNA polymerase. The loop areas, illustrated in FIGS. 27-30, can be comprised of; single-stranded DNA, one or more spacer molecules (e.g. Spacer 18, illustrated in FIG. 31), combinations thereof and the like, that allow flexibility for the intramolecular hybridization to occur. The sequence that is cleaved from the 3′ end of each oligonucleotide species has a lower Tm than the intact oligonucleotide species. The temperature at which the oligonucleotides are used in the subsequent amplification assay is higher than the temperature at which the cleaved 3′ end or cleaved reverse complement will anneal. Thus the cleaved fragments will not interfere with the amplification reactions.

In some embodiments, a thermostable nicking endonuclease can be used in place of a restriction endonuclease, as illustrated in FIG. 30A. A nicking enzyme cuts, in a sequence specific manner, only one of the two strands of double stranded DNA. Two non-limiting examples of thermostable nicking enzymes are Nb.BamI and Nb.BsrDI. Nb.BamI and Nb.BsrDI have an optimal enzymatic function temperature of 65 C, thus allowing design of oligonucleotide species that anneal at 65 C or below. Nb.BsmI cleaves the sequence 5′-NGCATTC-3′ into 5′-NG-3′ and 5′-CATTC-3′. Thus the oligonucleotide species sequence terminates at the 3′ with 5′-NG-3′ (any combination of A, C, G or T at the penultimate base and a G as the 3′ base). Nb.BsrDI cleaves the sequence 5′-NNCATTGC-3′ into 5′-NNCATTGC-3′ and 5′-NNCATTGC-3′. Thus the oligonucleotide species sequence terminates at the 3′ with 5′-NN-3′ (any dinucleotide sequence comprised of any combination of A, C, G or T). Removal of the block using a nicking endonuclease is illustrated in FIG. 30B. A single stranded cut (e.g., “nick”) cleaves the oligonucleotide species, in a sequence specific manner, removing the block and leaving a DNA polymerase extendable 3′ hydroxyl. The sequence that is cleaved from the 3′ end of each oligonucleotide species has a lower Tm than the intact oligonucleotide species. The temperature at which the oligonucleotides are used in the subsequent amplification assay is higher than the temperature at which the cleaved 3′ end or cleaved reverse complement will anneal. Thus the cleaved fragments will not interfere with the amplification reactions. In designing oligonucleotide species compositions for use with nicking enzymes, the 3′ end of the oligonucleotide must contain the 5′ portion of the nicking endonuclease recognition sequence, as illustrated in FIGS. 30A and 30B.

FIG. 32 illustrates a method for amplifying and capturing and/or detecting a target nucleic acid using a pair of 3′ blocked linear oligonucleotide species having complementary 3′ ends. The method can also make use of the J-hook oligonucleotide species compositions and the appropriate endonuclease, as described above (see FIGS. 27-30).

3′ blocked linear oligonucleotide species compositions having complementary 3′ ends are pairs of oligonucleotides that comprise a forward and reverse set of oligonucleotides that can be extended by a DNA polymerase, after the 3′ block is removed, leaving a free 3′ hydroxyl. The complementary 3′ ends of each oligonucleotide pair forms a first, internal restriction endonuclease recognition site when annealed (e.g., thermostable restriction or AP endonuclease, for example) that does not occur in the template DNA. The first restriction endonuclease cleavage site will be regenerated if the forward and reverse oligonucleotides reanneal, but not if the forward and reverse oligonucleotides anneal to the target nucleic acid, thus the presence of the first restriction endonuclease in the extension or amplification reactions eliminates “primer-dimer” artifacts. The complementary 3′ ends also may include additional nucleotides for increased binding efficiency and thermostability.

A capture agent and/or detectable moiety is linked to a second restriction endonuclease cleavage site located at the 5′ end of the forward oligonucleotide of the set. When configured in this manner, at least two rounds of extension or amplification are required before the second restriction endonuclease cleavage site is generated, allowing the release of the capture agent and/or detectable moiety by cleavage with the second restriction endonuclease. Therefore, the compositions described in this example also may be used to monitor the status of a reaction, in some embodiments.

As illustrated in FIG. 32, the oligonucleotide species compositions pairs are contacted with target nucleic acid and components necessary to support function of the added thermostable enzymes (e.g., polymerases and/or endonucleases) under hybridization conditions, and the mixture incubated to allow cleavage of the endonuclease cleavage sites, and annealing of the unblocked oligonucleotides. The reactions are allowed to proceed under extension conditions. The reactions generate amplicons that include the newly generated second endonuclease cleavage site. Cleavage of the second endonuclease cleavage site releases the capture agent and/or detectable moiety. In some embodiments, hybridization conditions, extension conditions, and cleavage conditions are substantially similar.

To minimize or eliminate the possibility of a DNA polymerase fill-in reaction, use of a restriction endonuclease cleavage site, for an enzyme that leaves a blunt ended cut or a 5′ overhang is preferred. The compositions described in this example can also be used in a fluorescent assay by adding a fluorescent moiety (for example, FAM) to the 5′ end of an oligonucleotide and a quencher to the 3′ end of the same oligonucleotide. In some embodiments, fluorescence can be doubled or two different types of fluorescence can be monitored, by labeling both the forward and reverse oligonucleotides with the same or different fluorescent moieties.

### Example 7

### Induced Nicking Activity

In some embodiments, an induced nicking function can be used to unblock a 3′ blocked J-hook oligonucleotide species composition pair or set. Restriction endonucleases are multimeric enzymes that cleave in a sequence specific manner on both strands of double stranded DNA. The thermostable “nicking enzymes”, Nb.BamI and Nb.BsrDI, are thermostable, engineered endonucleases, which have been mutationally altered to inhibit the ability of one of the enzyme subunits to cleave DNA. The result is an artificially created thermostable nicking enzyme.

To eliminate the need for artificially engineered nicking enzymes, oligonucleotide species compositions containing non-cleavable nucleotide analogs can be created to screen for enzymes that can be induced to nick (e.g., cleave a single strand in a double stranded DNA) double stranded DNA in the presences. The screening procedure is one easily carried and uses routine laboratory protocols. Oligonucleotide species are synthesized in pairs with complementary sequences, where one member of the pair incorporates one or more non-cleavable nucleotide analogs. In some embodiments, a detectable feature or capture agent or both, also can be incorporated into the screening oligonucleotide. The templates are incubated under cleavage or amplification conditions in the presence of the restriction endonuclease, and the reaction monitored by capture of the fragment carrying the capture agent, or by detection of the detectable feature. Presence of the fragment of the correct size or detection of the detection feature, indicates that the restriction endonuclease was able to be induced to “nick” a single strand of DNA, when non-cleavable nucleotide analogs were incorporated into the cleavage site.

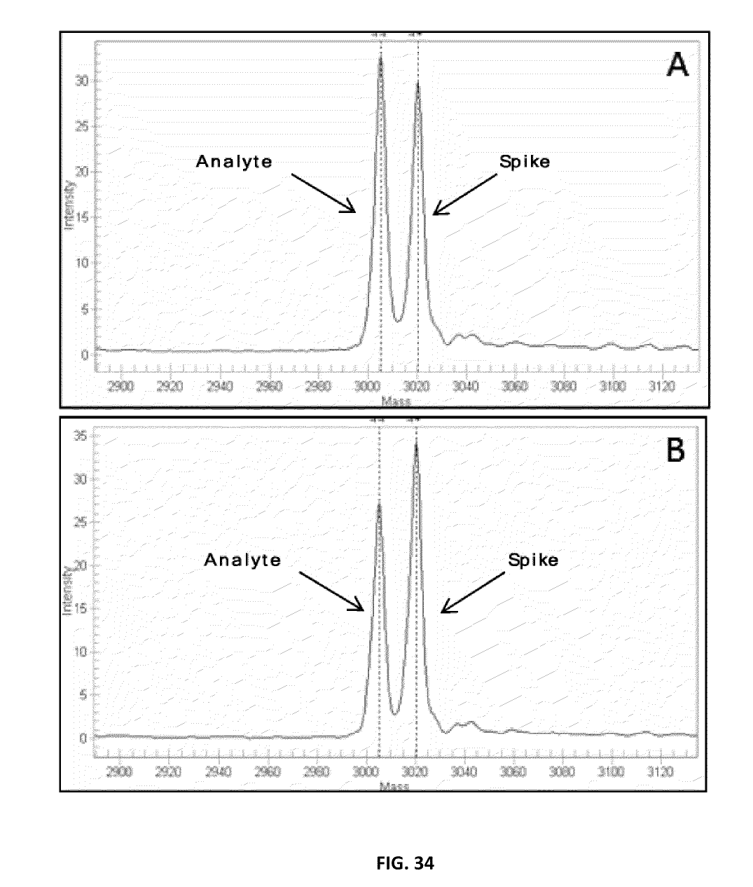

Identification of thermostable restriction endonucleases that can be induced to nick double stranded oligonucleotide species templates would allow greater design flexibility for oligonucleotide species compositions described herein. The oligonucleotide species compositions containing non-cleavable nucleotide analogs are illustrated in FIG. 33. The oligonucleotide species compositions can be designed as duplex pairs (e.g., 4 oligonucleotides per set as described in Example 5) as illustrated in FIG. 33, or as J-hook oligonucleotide species composition pairs (not shown). The restriction endonuclease sites are formed by annealing complementary regions in the oligonucleotide species compositions. In one of the complementary regions, a non-cleavable nucleotide analog is incorporated into the restriction endonuclease sequence. This will allow cleavage of the natural nucleotide, but the non-cleavable nucleotide analog will not be cut, generating an induced sequence specific nick. A non-limiting example is the use of a phosphorothioate bond to substitute a sulfur atom for a non-bridging oxygen in the phosphate backbone of an oligonucleotide, which renders the internucleotide linkage resistant to nuclease degradation. Phosphorothioates introduced internally can limit attack by endonucleases. The induced nicking method may be used in place of any of the examples described above that were designed to use a thermostable AP endonuclease.

### Example 8

### Experimental Results of Blocked Oligonucleotide Species Experiments Using Oligonucleotide Species Compositions Containing BstUI or BsaAI Thermostable Restriction Endonucleases

Compositions using BstUI cleavage site containing blocked oligonucleotide species compositions.

The restriction endonuclease BstUI recognizes the sequence CGCG and has an optimal temperature of 60 C. When DNA is cleaved by BstUI restriction endonuclease the cleavage event leaves the dinucleotide sequence CG at the 3′ end of the upstream fragment. The 3′ end contains a free 3′ hydroxyl that can subsequently be extended by a polymerase. Blocked oligonucleotide compositions, as described herein, were designed against the Homo sapiens SRY gene for sex determining region Y, isolate ADT3 (GenBank AM884751.1):

| | |
|---|---|
| 1 | ATGCAATCATATGCTTCTGCTATGTTAAGCGTACTCAACAGCGATGATTACAGTCCAGCT |
| 61 | GTGCAAGAGAATATTCCCGCTCTCCGGAGAAGCTCTTCCTTCCTTTGCACTGAAAGCTGT |
| 121 | AACTCTAAGTATCAGTGTGAAACGGGAGAAAACAGTAAAGGCAACGTCCAGGATAGAGTG |
| 181 | AAGCGACCCATGAACGCATTCATCGTGTGGTCTCGCGATCAGAGGCGCAAGATGGCTCTA |
| 241 | GAGAATCCCAGAATGCGAAACTCAGAGATCAGCAAGCAGCTGGGATACCAGTGGAAAATG |
| 301 | CTTACTGAAGCCGAAAAATGGCCATTCTTCCAGGAGGCACAGAAATTACAGGC CATGCAC |
| 361 | AGAGAGAAATACCCGAATTATAAGTATCGACCTCGTCGGAAGGCGAAGATGCTGCCGAAG |
| 421 | AATTGCAGTTTGCTTCCCGCAGATCCCGCTTCGGTACTCTGCAGCGAAGTGCAACTGGAC |
| 481 | AACAGGTTGTACAGGGATGACTGTACGAAAGCCACACACTCAAGAATGGAGCACCAGCTA |
| 541 | GGCCACTTACCGCCCATCAACGCAGCCAGCTCACCGCAGCAACGGGACCGCTACAGCCAC |
| 601 | TGGACAAAGCTGTAG |

The BstUI cleavage sequence CG/CG occurs once in the SRY sequence and the occurrences are underlined in the example. Oligonucleotide species compositions were designed to avoid BstUI cleavage sites in the resultant amplicon. Multiple occurrences of CG are underlined in the SRY sequence and are potential locations for placement of the 3′ end of primers. The Pvu II cleavage sequence CAG/CTG occurs twice in the SRY sequence and the occurrences are underlined in the example. Oligonucleotide species compositions were designed to avoid Pvu II cleavage sites in the resultant amplicon.

BstUI Blocked Oligo Sequences:

| | |
|---|---|
| Forward Primer: | |
| SRY.BstUI.f1 | /5BioTEG/AAAAACAGCTGGTGAAGCGACCCA |
| | TGAACGCGTGTGGTCTCGCGATCA/3SpC3/ |
| Reverse Primer: | |
| SRY.BstUI.r1 | TGATCGCGAGACCACACGCGTTCATGGGTCG |
| | CTTCAC/3SpC3/ |
| Cleaved Analyte | /5BioTEG/AAAAACAG |
| Detected on | |
| MALDI: | |

The intact probe has a mass of 15,543 daltons. The cleaved tag or analyte has a mass of 3005.2 daltons. The internal spike has a mass of 3020.3 daltons. The region of sequence that is complementary to the target sequence and that will act as the extension oligonucleotide for the amplification reaction is underlined. The oligonucleotide composition containing the forward extension oligonucleotide sequence also contains a Pvu II 5′ tag sequence and the reverse complement sequence of reverse extension oligonucleotide. The oligonucleotide species composition containing the reverse extension oligonucleotide also contains the reverse complement sequence of the forward extension oligonucleotide. Hybridization of these oligonucleotides in the PCR creates a BstUI cleavage site. Once cleaved, the functional (e.g., deblocked) oligonucleotide species compositions participate in PCR amplification of target sequence. There is one set of oligonucleotides for the BstUI blocked oligonucleotide.

Control Extension Oligonucleotide Sequences are as Follows:

| | |
|---|---|
| Forward Primer: | |
| SRY.f1.PvuII | /5BioTEG/AAAAACAGCTGCGATCAGAGGCG |
| | CAAGATG |
| Reverse Primer: | |
| SRY.r1.f | GCTGATCTCTGAGTTTCGCATTCTG |
| Cleaved Analyte | /5BioTEG/AAAAACAG |
| Detected on MALDI: | |

The intact control probe has a mass of 9876.7 daltons. The cleaved tag or analyte has a mass of 3005.2 daltons. The oligonucleotide composition containing the forward extension oligonucleotide sequence also contains a Pvu II 5′ tag sequence. The control reaction confirms that the PCR and Pvu II cleavage were effective under the thermocycling protocols used for the blocked oligonucleotide species compositions.

The assays were amplified in 20 μL reactions with the following final concentrations: 1× buffer (50 mM Tris-HCl, 4 mM (NH4)2SO4, 10 mM KCl, 4 mM MgCl), 125 μM dATP, 125 μM dCTP, 125 μM dGTP, 125 μM dTTP, 2 units Roche FastStart DNA polymerase, 300 nM forward oligo, 300 nM reverse oligo, 20 nM spike oligo, 7.5 ng human genomic DNA, 5 units Pvu II restriction endonuclease and 4 units BstUI restriction endonuclease.

An internal standard or spike was added to the PCR master mix. The spike has a mass 15 daltons greater than the analyte. The internal standard can be used to normalize for differences in pipetting of PCR reactions, loss through post-PCR handling such as purification with Streptavidin-coated paramagnetic beads and spotting onto MALDI chips, and differences in MALDI instrument performance. Peak area response ratio can be calculated for the analyte and the corresponding spike (Bruenner et al 1996). The internal spike has a mass of 3020.3 daltons.

| | |
|---|---|
| Spike added at PCR: | |
| SRY1.Spike1L | /5BioTEG/AAAGAAAT |

Oligo sequences which are annotated with /5BioTEG/ contain a biotin attached to the 5′ end of the oligo by an extended 15-atom spacer arm. Oligo sequences which are annotated with /3SpC3/ contain a 3′ C3 Spacer. In this example use of a 3′ C3 Spacer (instead of a 3′ hydroxyl) prevents DNA polymerase from extending the hybridization oligo. Other moieties may be substituted at the 3′ terminus can prevent DNA polymerase from extending the oligo. Such moieties may include but are not limited to 3′ Amino Modifiers, 3′ Biotin, 3′ Biotin TEG, 3′ Cholesteryl-TEG, 3′ Digoxigenin, 3′ Thiol, 3′ Inverted dT or 3′ Phosphate.

BstUI Reactions were Subjected to a Thermocycling Protocol of:
90 C for 5 sec
60 C for 1 hr (optimal temperature for BstUI restriction endonuclease)
95 for 3 min
95 C for 10 sec, 60 C for 10 sec and 72 C for 20 sec for 35 cycles
37 C for 1 hr (optimal temperature for Pvu II restriction endonuclease)

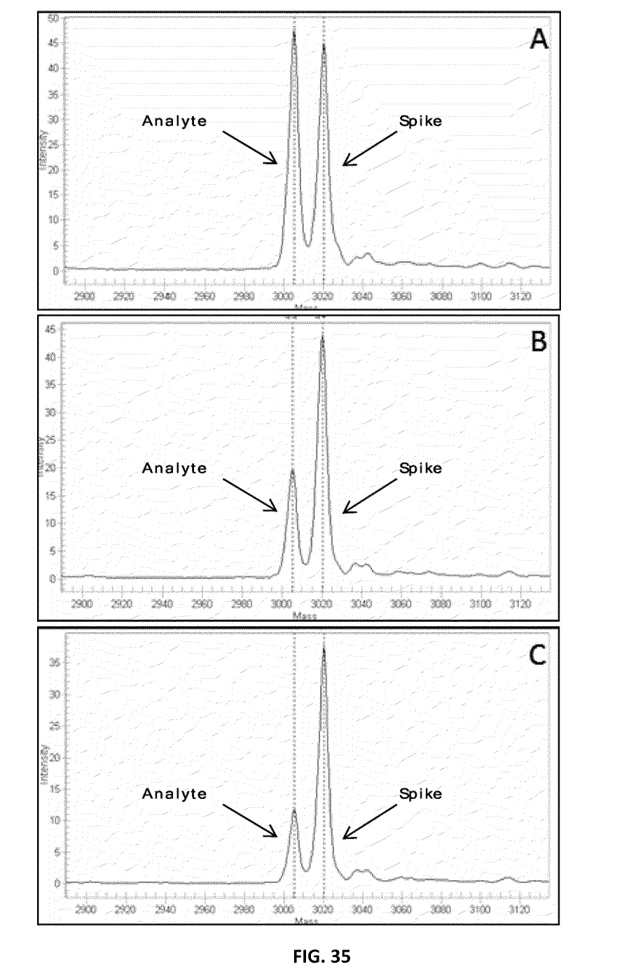

Reactions were subsequently purified by capture of the 5′ biotin moiety with Streptavidin-coated paramagnetic beads. FIGS. 34A and 34B illustrate the results of MALDI mass spectrometry detection of oligonucleotides extended from cleaved blocked oligonucleotide species compositions using extension and amplification methods described herein. FIG. 34A shows the 5′ Pvu II tag spectra for a control reaction with unblocked oligonucleotide species compositions. The control reaction confirms that the PCR and Pvu II cleavage were effective under the thermocycling protocols used for the blocked oligonucleotide species compositions. FIG. 34B show the 5′ Pvu II tag spectra for a reaction with the BstUI blocked oligonucleotide species compositions as described herein. The analyte peak is present, indicating that the oligonucleotide species compositions were unblocked by the BstUI restriction endonuclease added to the PCR. The spectra in all panels includes a reference Spike peak added during PCR setup.

Compositions using BstUI cleavage site containing blocked oligonucleotide species compositions.

The restriction endonuclease BsaAI recognizes the sequence YACGTR and will cut at any of the 4 sequences TACGTA, CACGTA, TACGTG or CACGTG. The enzyme has an optimal temperature of 50 C. When DNA is cleaved by BsaAI restriction endonuclease the cleavage event leaves the trinucleotide sequence TAC or CAC at 3′ end of the upstream fragment. The 3′ end contains a free 3′ hydroxyl that can subsequently be extended by a polymerase. Blocked primer oligonucleotide species were designed against the Homo sapiens SRY gene for sex determining region Y, isolate ADT3 (GenBank AM884751.1):

| | |
|---|---|
| 1 | ATGCAATCATATGCTTCTGCTATGTTAAGCGTACTCAACAGCGATGATTACAGTCCAGCT |
| 61 | GTGCAAGAGAATATTCCCGCTCTCCGGAGAAGCTCTTCCTTCCTTTGCACTGAAAGCTGT |
| 121 | AACTCTAAGTATCAGTGTGAAACGGGAGAAAACAGTAAAGGCAACGTCCAGGATAGAGTG |
| 181 | AAGCGACCCATGAACGCATTCATCGTGTGGTCTCGCGATCAGAGGCGCAAGATGGCTCTA |
| 241 | GAGAATCCCAGAATGCGAAACTCAGAGATCAGCAAGCAGCTGGGATACCAGTGGAAAATG |
| 301 | CTTACTGAAGCCGAAAAATGGCCATTCTTCCAGGAGGCACAGAAATTACAGGCCATGCAC |
| 361 | AGAGAGAAATACCCGAATTATAAGTATCGACCTCGTCGGAAGGCGAAGATGCTGCCGAAG |
| 421 | AATTGCAGTTTGCTTCCCGCAGATCCCGCTTCGGTACTCTGCAGCGAAGTGCAACTGGAC |
| 481 | AACAGGTTGTACAGGGATGACTGTACGAAAGCCACACACTCAAGAATGGAGCACCAGCTA |
| 541 | GGCCACTTACCGCCCATCAACGCAGCCAGCTCACCGCAGCAACGGGACCGCTACAGCCAC |
| 601 | TGGACAAAGCTGTAG |

The BsaAI restriction endonuclease cleavage sequences TACGTA, CACGTA, TACGTG or CACGTG do not occur in the SRY sequence. Multiple occurrences of CAC or TAC are underlined in the example and are potential locations for the placement of 3′ end of oligonucleotide sequences. The Pvu II restriction endonuclease cleavage sequence CAG/CTG occurs twice in the SRY sequence and the occurrences are underlined in the example. Oligonucleotide species compositions were designed to avoid Pvu II cleavage sites in the resultant amplicon. Oligonucleotide species compositions were designed for two separate amplicons in two separate assays with BsaAI blocked oligonucleotide sequences.

| | |
|---|---|
| Blocked Oligo Set #1 | |
| Forward Primer: | |
| SRY.BsaAI.f1 | /5BioTEG/AAAAACAGCTGGGCCATGC |
| | ACAGAGAGAAATACGTATCGACCTCGTC |
| | GGAAGG/3SpC3/ |
| Reverse Primer: | |
| SRY.BsaAI.r1 | CCTTCCGACGAGGTCGATACGTATTTCT |
| | CTCTGTGCATGGCC/3SpC3/ |
| Blocked Oligo Set #2 | |
| Forward Primer: | |
| SRY.BsaAI.f2 | /5BioTEG/AAAAACAGCTGAAGCTCTT |
| | CCTTCCTTTGCACGTAAAGGCAACG |
| | TCCAGGATAG/3SpC3/ |
| Reverse Primer: | |
| SRY.BsaAI.r2 | CTATCCTGGACGTTGCCTTTACGTGCAA |
| | AGGAAGGAAGAGCTT/3SpC3/ |

The region of sequence that is complementary to the target sequence and that will act as the extension oligonucleotides for the amplification reaction is underlined in each oligonucleotide species compositions. The oligonucleotide species compositions containing the forward extension oligonucleotide sequence also contains a Pvu II 5′ tag sequence and the reverse complement sequence of reverse extension oligonucleotide. The oligonucleotide species compositions containing the reverse extension oligonucleotide also contain the reverse complement sequence of the forward extension oligonucleotide. Hybridization of these oligonucleotide species compositions in the PCR creates a BsaAI cleavage site. Once cleaved the released extension oligonucleotides participate in PCR amplification of target sequence.

The assay was performed in a 20 μL reaction with the following final concentrations: of 1× buffer (50 mM Tris-HCl, 4 mM (NH4)2SO4, 10 mM KCl, 4 mM MgCl), 125 μM dATP, 125 μM dCTP, 125 μM dGTP, 125 μM dTTP, 2 units Roche FastStart DNA polymerase, 300 nM forward oligo, 300 nM reverse oligo, 20 nM spike oligo, 7.5 ng human genomic DNA, 5 units Pvu II restriction endonuclease and 2 units BsaAI restriction endonuclease.

An internal standard or spike was added to the PCR master mix. The spike has a mass 15 daltons greater than the analyte. The internal standard can be used to normalize for differences in pipetting of PCR reactions, loss through post-PCR handling such as purification with Streptavidin-coated paramagnetic beads and spotting onto MALDI chips, and differences in MALDI instrument performance. Peak area response ratio can be calculated for the analyte and the corresponding spike (Bruenner et al 1996). The internal spike has a mass of 3020.3 daltons.

Oligonucleotide sequences which are annotated with /5BioTEG/ contain a biotin attached to the 5′ end of the oligonucleotide by an extended 15-atom spacer arm. Oligonucleotide sequences which are annotated with /3SpC3/ contain a 3′ C3 Spacer. In this example use of a 3′ C3 Spacer (instead of a 3′ hydroxyl) prevents DNA polymerase from extending the extension oligonucleotide. Other moieties may be substituted at the 3′ terminus that also can prevent DNA polymerase from extending an oligonucleotide. Such moieties may include but are not limited to 3′ Amino Modifiers, 3′ Biotin, 3′ Biotin TEG, 3′ Cholestery1-TEG, 3′ Digoxigenin, 3′ Thiol, 3′ Inverted dT or 3′ Phosphate.

Reactions were Subjected to a Thermocycling Protocol of:
90 C for 5 sec
50 C for 1 hr (optimal temperature for BsaAI restriction endonuclease)
95 for 3 min
95 C for 10 sec, 60 C for 10 sec and 72 C for 20 sec for 35 cycles
37 C for 1 hr (optimal temperature for Pvu II restriction endonuclease)

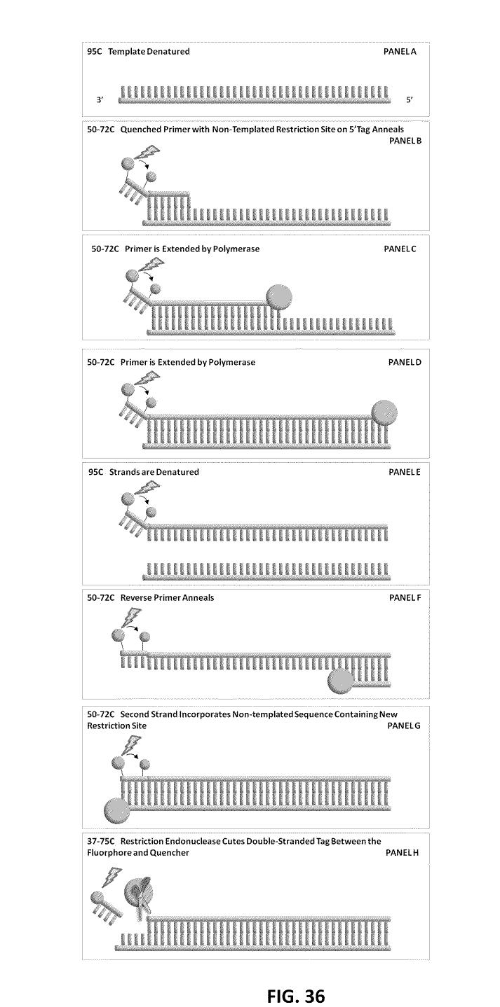

Reactions were subsequently purified by capture of the 5′ biotin moiety with Streptavidin-coated paramagnetic beads. FIGS. 35A-35C illustrate the results of MALDI mass spectrometry detection of oligonucleotides extended from cleaved blocked oligonucleotide species compositions using extension and amplification methods described herein. FIG. 35A shows the 5′ Pvu II tag spectra for a control reaction with unblocked oligonucleotide species compositions. The control reaction confirms that the PCR and Pvu II cleavage were effective under the thermocycling protocols used for the blocked oligonucleotide species compositions. FIG. 35B shows the 5′ Pvu II tag spectra for a reaction with the blocked oligonucleotide species composition pair, Set #1 SRY.BsaAI.f1 and SRY.BsaAI.r1, as described herein. The analyte peak is present, indicating that the pair of oligonucleotide species compositions were unblocked by the BsaAI restriction endonuclease added to the PCR reaction. FIG. 35C shows the 5′ Pvu II tag spectra for a reaction with the blocked oligonucleotide species composition pair, Set #2 SRY.BsaAI.f2 and SRY.BsaAI.r2, as described herein. The analyte peak is present, indicating that the oligonucleotide species compositions were unblocked by the BsaAI restriction endonuclease added to the PCR. The spectra in all panels includes a reference Spike peak added during PCR setup.

### Example 9

### Partial List of Restriction Endonucleases that are not Heat Activated

Provided below is a table listing non-limiting examples of thermostable restriction endonucleases (table divided into 2 parts). The data presented below is available at World Wide Web URL neb.com. The thermostability, defined as the heat tolerance half-life and described above, has been investigated for some of the enzymes presented below. The heat-tolerance half life is an important consideration when designing thermocycling profiles, to minimize complete inactivation of the restriction endonucleases. Some heat tolerant enzymes can refold after several denaturation cycles and retain at least 50% of their activity. This allows for multiple rounds of amplification. Other heat tolerant enzymes lose greater than 50% of their activity in only one or a few rounds of amplification. Further investigation is being conducted on the heat tolerant half life of thermostable enzymes. The embodiments described herein can be adapted to make use of any heat tolerant (e.g., thermostable) restriction endonuclease, and therefore are not limited by the enzymes included in the table below.

| | | | | | | Thermopol | | | | |
|---|---|---|---|---|---|---|---|---|---|---|
| | % Activity in NEB buffers | | | | Taq PCR | PCR | Heat | Rxn | | |
| Enzyme | 1 | 2 | 3 | 4 | Buffer | Buffer | Inactivated | Temp | Overhang | Cat. # |
| AclI | 10 | 10 | 0 | 100 | +++ | +++ | No | 37 | 3 | R0598S |
| ApaLI | 100 | 100 | 10 | 100 | +++ | +++ | No | 37 | 3 | R0507S |
| ApeKI | 25 | 75 | 100 | 50 | <++@75 C. | <++@75 C. | No | 75 | 3 | R0643S |
| BamHI | 75 | 100 | 100 | 100 | +++ | +++ | No | 37 | 3 | R0136T |
| BamHI- | 100 | 50 | 10 | 100 | +++? | +++? | No | 37 | 3 | R3136S |
| HF ™ | | | | | | | | | | |
| BclI | 50 | 100 | 100 | 75 | +++@50 C. | +++@50 C. | No | 50 | 3 | R0160S |
| BgIII | 10 | 75 | 100 | 10 | <+ | <+ | No | 37 | 3 | R0144S |
| BlpI | 50 | 100 | 10 | 100 | <++ | <++ | No | 37 | 3 | R0585S |
| BsaAI | 100 | 100 | 100 | 100 | ++ | +++ | No | 37 | B | R0531S |
| BsaXI | 75 | 100 | 10 | 100 | ++ | +++ | No | 37 | B | R0609S |
| BsiHKAI | 50 | 100 | 100 | 100 | <+@65 C. | −@65 C. | No | 65 | 5 | R0570S |
| BsoBI | 10 | 100 | 100 | 50 | +++ | +++ | No | 37 | 3 | R0586S |
| BsrFI | 10 | 100 | 100 | 100 | <+ | <+ | No | 37 | 3 | R0562S |
| BstBI | 75 | 50 | 25 | 100 | +++@65 C. | +++@65 C. | No | 65 | 3 | R0519S |
| BstEII | 50 | 75 | 100 | 75 | +++@60 C. | +++@60 C. | No | 60 | 3 | R0162S |
| BstNI | 10 | 100 | 100 | 75 | +++@60 C. | +++@60 C. | No | 60 | 3 | R0168S |
| BstUI | 100 | 100 | 50 | 100 | +++@60 C. | +++@60 C. | No | 60 | B | R0518S |
| BstZ17I | NR | NR | 100 | 100 | +++ | +++ | No | 37 | B | R0594S |
| BtsCI | 50 | 100 | 50 | 100 | +++ | +++ | No | 50 | 5 | R0647S |
| CviQI | 75 | 100 | 100 | 75 | +@25 C. | +++@25 C. | No | 25 | 3 | R0639S |
| HpaI | 25 | 50 | 10 | 100 | +++ | +++ | No | 37 | B | R0105S |
| KpnI | 100 | 75 | 0 | 50 | +++ | +++ | No | 37 | 5 | R0142S |
| MwoI | 10 | 75 | 100 | 75 | +++@60 C. | +++@60 C. | No | 60 | 5 | R0573S |
| NciI | 100 | 25 | 10 | 100 | +++ | +++ | No | 37 | 3 | R0196S |
| PaeR7I | 25 | 100 | 10 | 100 | +++ | +++ | No | 37 | 3 | R0177S |
| PhoI | 50 | 50 | 100 | 75 | ++@75 C. | ++@75 C. | No | 75 | B | R0705S |
| PpuMI | 0 | 25 | 0 | 100 | +++ | +++ | No | 37 | 3 | R0506S |
| PvuII | 100 | 100 | 100 | 100 | +++ | +++ | No | 37 | B | R0151T |
| SfiI | 0 | 100 | 10 | 100 | <+@50 C. | +++@50 C. | No | 50 | 5 | R0123S |
| SfoI | 25 | 100 | 50 | 100 | +++ | +++ | No | 37 | B | R0606S |
| SmII | 25 | 75 | 25 | 100 | <+@55 C. | <+@55 C. | No | 55 | 3 | R0597S |
| TfiI | 100 | 100 | 100 | 100 | <++@65 C., − | <++@65 C., − | No | 65 | 3 | R0546S |
| Tsp509I | 100 | 100 | 100 | NR | @75 C.+++ | @75 C.+++ | No | 65 | 3 | R0576S |
| TspMI | 50 | 75 | 50 | 100 | +++@75 C. | +++@75 C. | No | 75 | 3 | R0709S |
| TspRI | 25 | 50 | 25 | 100 | + | + | No | 65 | 5 | R0582S |
| ZraI | 100 | 25 | 10 | 100 | +++ | +++ | No | 37 | B | R0659S |

### Example 10

### Oligonucleotide Species Composition Adapted for use in Fluorescence Based Detection Methods

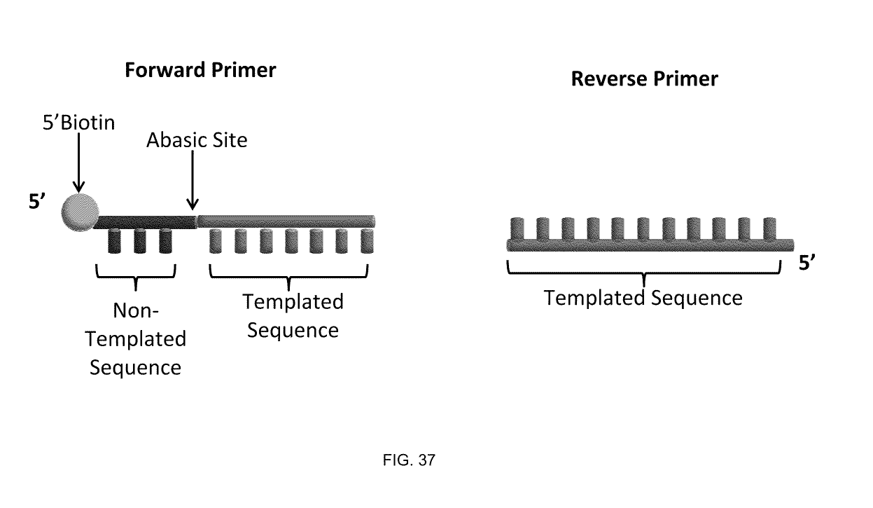

FIG. 36 illustrates a method for generating a fluorescent signal from an oligonucleotide species composition containing a thermostable restriction endonuclease and requiring at least two rounds of oligonucleotide extension. The method steps are similar to those described above for FIG. 10, in Example 3, and will therefore not be described here. The difference between the two examples resides in the substitution of a detectable fluorescent feature for the capture agent illustrated in FIG. 10 of Example 3. The embodiment presented in FIG. 36 makes use of a signal-pair fluorescent agent (e.g., emitter and quencher, or in the case of FRET, exciter and emitter), however one of skill will appreciate that any detectable feature or fluorescent feature, that can be adapted for use with the compositions described herein, can be substituted for the signal-pair detectable feature presented in FIG. 36. Signal-pair detectable agents suitable for use with the compositions and methods described herein are described above.

In the embodiment present in FIG. 36, the quencher is incorporated 3′ of the restriction endonuclease cleavage site. This allows activation of the detectable feature after the restriction endonuclease cleavage site is generated from at least two rounds of oligonucleotide extension. That is, extension must occur such that an extended product from the 5′ tagged forward oligonucleotide is generated, which then is annealed by a reverse oligonucleotide and extended, thereby generating a double stranded restriction endonuclease recognition site. Cleavage under cleavage conditions liberates the tag, and separates the quencher from the fluorophore, thereby allowing detection of the detectable feature.

### Example 11

### Sulfolobus DNA Polymerase IV and Tth Endonuclease Internal Hybridization Probe Assay

A polymerase capable of synthesizing DNA across a variety of DNA template lesions may be incorporated into an assay described herein, in certain embodiments. Sulfolobus DNA Polymerase IV is a non-limiting example of a thermostable Y-family lesion-bypass DNA Polymerase that efficiently synthesizes DNA across a variety of DNA template lesions.

Translesion-Synthesizing DNA Polymerase

DNA strands occasionally contain ‘lesions’ caused by factors such as uv light, radiation, cell metabolic by-products or exogenous chemicals. As a result of the damage, DNA bases sometimes become oxidized, alkylated, hydrolyzed (deaminated, depurinated and depyrimidated), mismatched or otherwise modified. Non-limiting examples of such lesions include abasic sites, thymine dimers, nicks and gaps, deaminated cytosine, 8-oxo-guanine and 8-oxo-7,8-dihydro-2′deoxyadenosine.

Replication of DNA can be stalled when a high-fidelity DNA polymerase encounters certain lesions in DNA strands. Non-limiting examples of high-fidelity DNA polymerase include Taq DNA polymerase and Pfu DNA polymerase. DNA damage that stalls high-fidelity DNA polymerases frequently is bypassed by the trans-lesion Y-family polymerases such as Sulfolobus DNA Polymerase IV (Dpo4).

Sulfolobus DNA Polymerase IV is a thermostable Y-family lesion-bypass DNA Polymerase that efficiently synthesizes DNA across a variety of DNA template lesions. Trans-lesion synthesis by Sulfolobus DNA Polymerase IV is enhanced by the presence of Mn²⁺ in the reaction. The enzyme is heat inactivated at 95 degrees Centigrade for 6 minutes. Sulfolobus DNA Polymerase IV can be less processive and less thermostable than thermostable DNA polymerases such as Taq DNA polymerase. Sulfolobus DNA Polymerase IV is commercially available (e.g., New England Biolabs (NEB), Ipswich, Mass., and Trevigen, Inc., Gaithersburg, Md.).

Tth Endonuclease IV

Tth Endonuclease IV is a thermostable apurinic/apyrimidinic (AP) endonuclease from Thermus thermophilus (New England Biolabs, Ipswich Mass.). It initiates removal of abasic moieties from damaged DNA. Endonuclease IV also is active on urea sites, base pair mismatches, flap and pseudo Y structures, and small insertions/deletions in DNA molecules. Tth endonuclease IV first nicks a DNA strand of double-stranded DNA at the lesions located closest to the 5′-end of the DNA molecule. Single-stranded DNA is cleaved with significantly lower efficiency than double-stranded DNA. Mg²⁺ or Mn²⁺ ions are required for enzyme activity and thermostability at elevated temperature is enhanced by the addition of 25 uM ZnCl₂. The enzyme has an optimal temperature range is 65 degrees Centigrade to 70 degrees Centigrade.

Sulfolobus DNA Polymerase IV and Tth Endonuclease Internal Hybridization Probe Assay

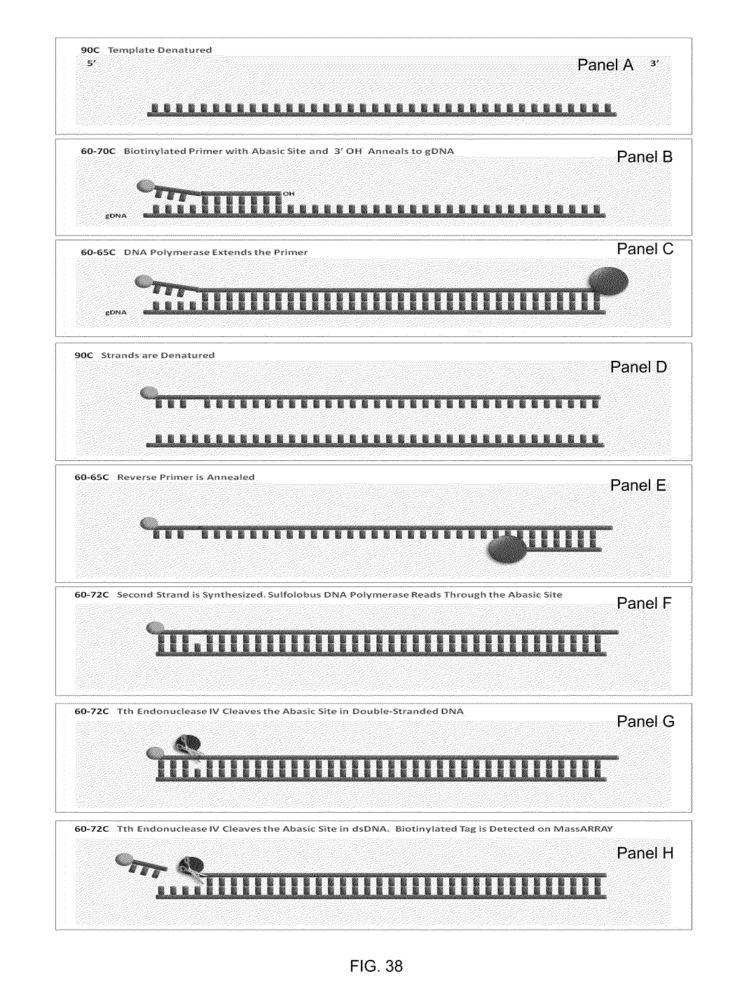

In some embodiments, Sulfolobus DNA polymerase IV and Tth endonuclease internal hybridization probe assay has a modified forward primer. In certain embodiments, the 5′sequence region is untemplated and the 3′ sequence region is templated. In some embodiments, the two sequence regions are separated by an internal abasic residue (see FIG. 37). The oligonucleotide may be tagged with a moiety that can be used in detection of the cleaved tag, in certain embodiments. Such a tag sometimes includes a 5′ biotin moiety that can be captured in a Streptavidin-biotin or similar purification method. The reverse primer is templated and unmodified, in some embodiments (see FIG. 37). The embodiment described in this Example does not have an internal hybridization probe.

When annealed to the denatured DNA template, the forward and reverse primers are extended by a DNA polymerase in the PCR reaction, in some embodiments. Trans-lesion DNA polymerases (e.g., Sulfolobus DNA Polymerase IV (NEB, Ipswich Mass.), Sulfolobus solfataricus DNA Polymerase IV (Dpo4) (Trevigen, Gaithersburg Md.), or any lesion-bypass DNA polymerases can incorporate a base across from a templated abasic site (or other lesion site), and permits polymerization past the abasic site (or other lesion site) introduced by the forward primer, in certain embodiments (see FIG. 38).

After PCR, the amplicon has an abasic site incorporated by the forward primer sequence. The opposite strand is synthesized and extended past the abasic site and the non-templated sequence introduced by the 5′ region of the forward primer (see FIG. 38). The addition of a thermostable abasic-cleaving enzyme such as Tth Endonuclease IV allows a specific tag to be cleaved from the double-stranded amplicon, in some embodiments. Any suitable method can be utilized to detect the cleaved tag. In certain embodiments, the cleaved tag is labeled with a 5′ biotin moiety which sometimes is captured and purified on a Streptavidin bead.

The trans-lesion Sulfolobus DNA Polymerase IV may be supplemented with a second DNA thermostable polymerase. Supplementing the trans-lesion Sulfolobus DNA polymerase IV may increase yield by assisting in the polymerization of templated sequences.

Materials and Methods

| Oligonucleotide | | |
|---|---|---|
| Name | Sequence | Comment |
| Reverse Primer | TGATCTCTGAGTTTCGCATTCTG | Unmodified oligonucleotide |
| Forward Primer | /5BioTEG/AAAAAA/idSp/CGATCA | 5′ modified with biotin, non-templated |
| | GAGGCGCAAGATG | sequence and abasic site. |
| MALDI Tag | /5BioTEG/AAAAAA/ | Cleaved from Forward Primer by Tth |
| | | Endonuclease |
| Passive | /5BioTEG/AAAAAA/3SpC3/ | Added to PCR Mix For MALDI |
| Reference Spike | | Quantitation |

Oligonucleotides used in representative assays are presented in the table above. The oligonucleotides are designed to amplify sequences in the human SRY gene. Oligonucleotides that include “/5BioTEG/” contain a biotin attached to the 5′ end of the oligonucleotide by an extended 15-atom spacer arm. Oligonucleotides that include “/idSp/” contain an internal abasic site, for example a 1′,2′-Dideoxyribose (dSpacer) moiety. Oligonucleotides that include “/3SpC3/” contain a 3-carbon spacer attached to the 3′ end of the oligonucleotide and render the oligonucleotide un-extendable by a DNA polymerase.

In some embodiments, a passive reference spike similar to, but different in mass from, the MALDI tag is added at a known concentration to the PCR reaction. The reference spike does not participate in the PCR reaction but is used as a reference for quantification using mass spectrometry. The cleaved tag is quantified by calculating the ratio of the cleaved tag to the passive reference tag, in certain embodiments. The ratio can be used as a control for efficiencies in PCR, sample purification, deposition on the MALDI chip matrix or detection by the MALDI instrument.

The assay can be performed in 25 microliter PCR reactions with the following final concentrations: 20 mM Tris-HCl, 10 mM (NH₄)₂SO₄, 10 mM KCl, 4 mM MgSO₄, 0.1% Triton X-100, 125 uM dATP, 125 uM dCTP, 125 uM dGTP, 125 uM dTTP, 0.5 nM MnCl₂, 25 uM ZnCl₂, 150 nm forward primer, 150 mM reverse primer, 25 nm internal reference spike, 0.5 units Tth endonuclease IV, 0.6 units Sulfolobus DNA Polymerase IV and 50 ng human genomic DNA.

Samples can be thermocycled as follows: one cycle at 90 degrees Centigrade for 5 sec; 35 cycles at 90 degrees Centigrade for 15 seconds, 60 degrees Centigrade for 10 seconds and 68 degrees Centigrade for 20 seconds; one cycle at 70 degrees Centigrade for 30 seconds. Samples can be held at 4 degrees Centigrade until they were processed for mass spectrometery analysis. After PCR, the biotin-containing oligonucleotides are purified by capture with Streptavidin beads (Dynabeads® MyOne™ Streptavidin C1, Invitrogen, Carlsbad Calif.).

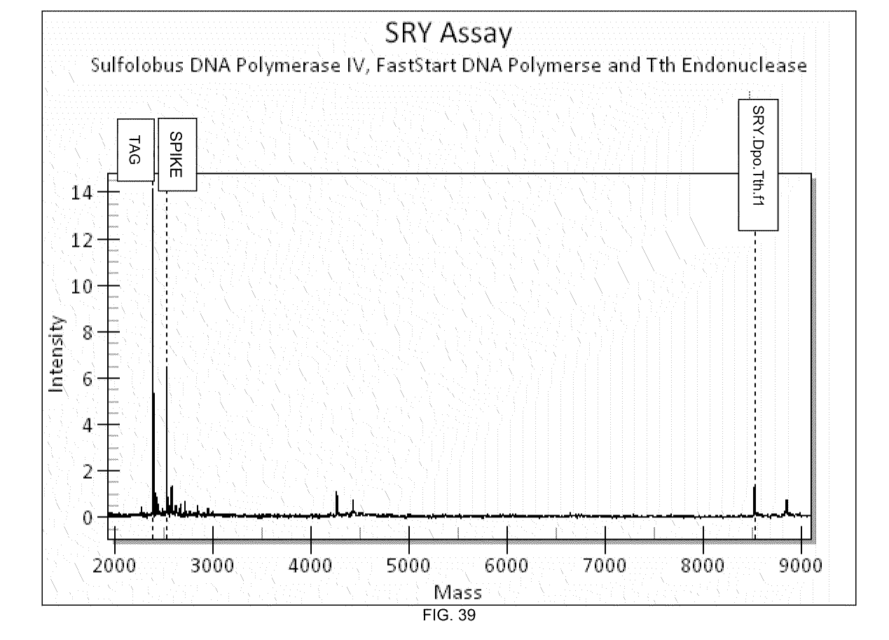

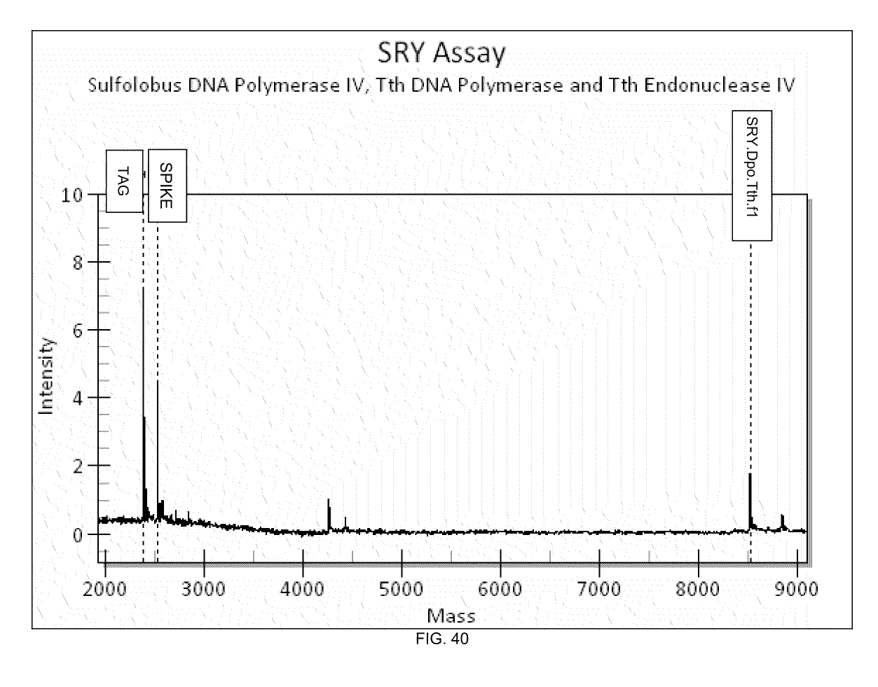

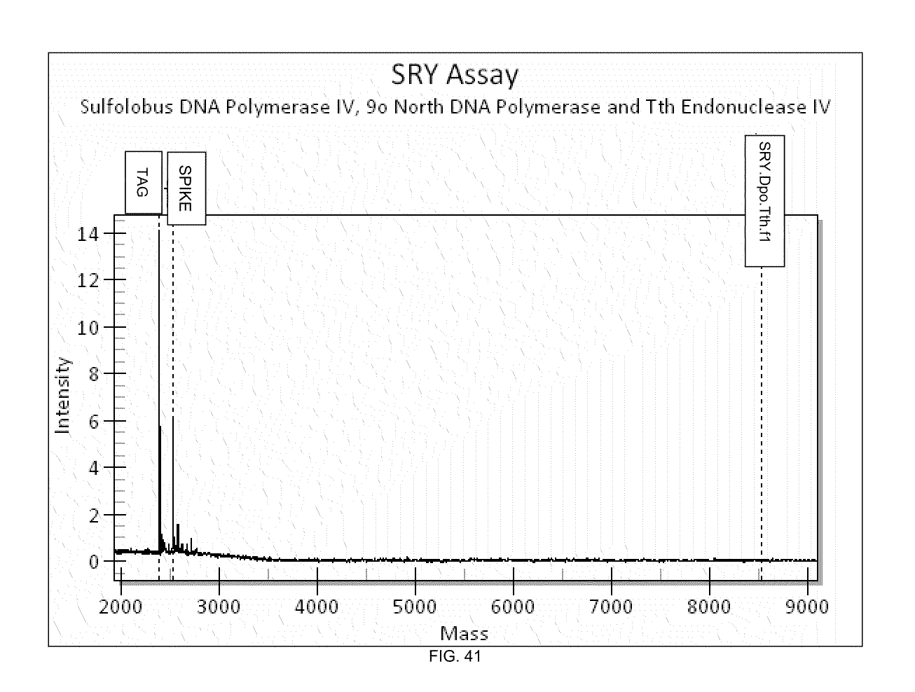

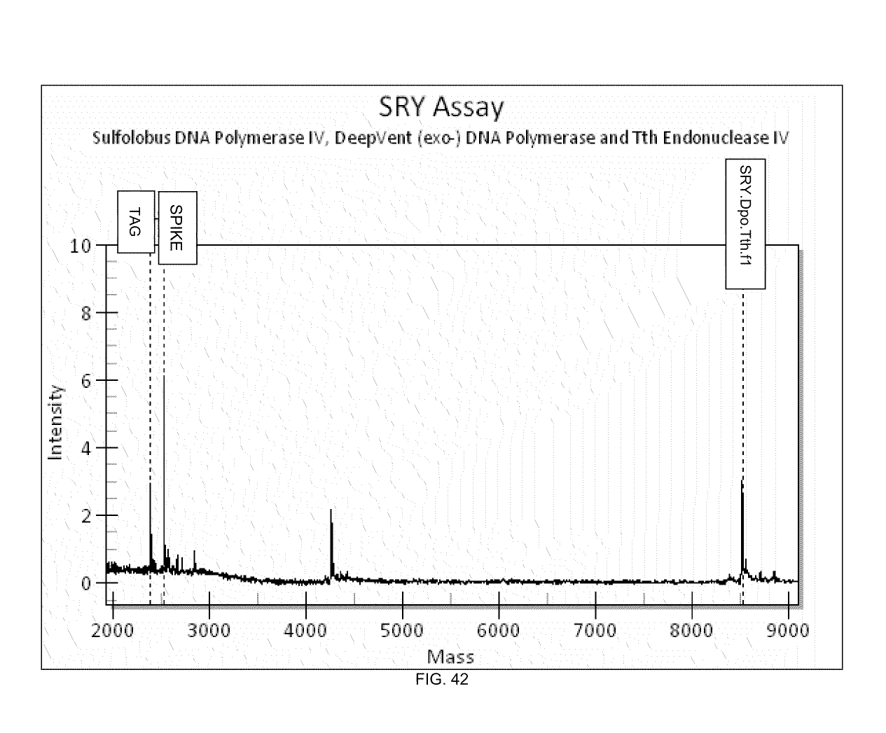

In some embodiments, reactions can contain a second DNA polymerase (e.g., Taq FastStart DNA polymerase (see FIG. 39), Tth DNA polymerase (see FIG. 40), 9°N™M DNA polymerase (see FIG. 41), Deep Vent_{R}™ (exo-) DNA polymerase (see FIG. 42)) to augment the processivity of the Sulfolobus DNA polymerase IV in polymerization of unmodified DNA bases. In FIGS. 39 to 42 cleaved tag is labeled “Tag”, the passive reference spike is labeled “Spike” and the uncleaved forward primer is labeled “SRY.Dpo.Tth.f1.” Each shows the presence of the cleaved tag and indicates cleavage by the Tth Endonuclease IV enzyme. Following is information for some of the polymerases.

Taq FastStart DNA polymerase is a modified recombinant Taq DNA Polymerase. It is inactive at temperatures below 75° C., but is activated by a 2- to 4-minute heat activation step at 95° C. Taq FastStart DNA polymerase was added at 1.0 unit per 25 microliter PCR reaction.

9°N™m DNA polymerase (NEB, Ipswich Mass.) is a thermophilic DNA polymerase that has been genetically engineered to have a decreased 3′ to 5′ proofreading exonuclease activity. The 9°N™m DNA polymerase was added at 0.4 units per 25 microliter PCR reaction.

Deep Vent_{R}™ (exo-) DNA polymerase (NEB, Ipswich Mass.) has been engineered to eliminate the 3′ to 5′ proofreading exonuclease activity associated with Deep Vent DNA Polymerase. Deep Vent (exo-) DNA polymerase was added at 0.4 units per 25 microliter PCR reaction.

Tth DNA polymerase (Promega, Madison Wis.) is a thermostable enzyme that possesses a 5′ to 3′ exonuclease activity and is used in recommended for use in PCR and reverse transcription reactions at elevated temperatures. Tth DNA Polymerase was added at 1.0 unit per 25 microliter PCR reaction.

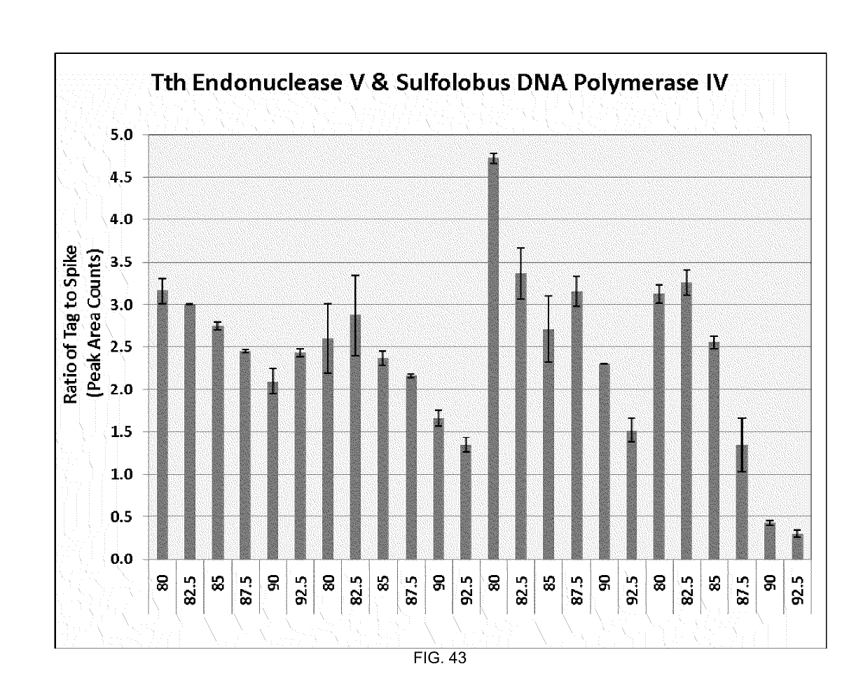

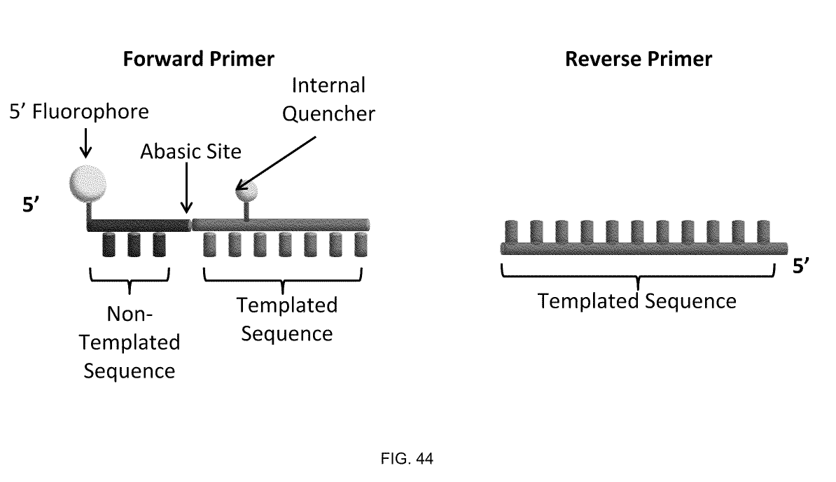

Sulfolobus DNA polymerase is not as thermostable as some other thermostable DNA polymerases. Sulfolobus DNA polymerase can be heat-inactivated after being held at 95 degrees Centigrade for 6 minutes. Altering denaturation time and temperature can be expected to affect yield. The effect of denaturation temperature on yield was evaluated and the data are presented in FIG. 43. The area under the peak of the MALDI tag was normalized to the area under the peak of the internal reference spike. In this experiment the yield of cleaved tag was reduced as the annealing temperature was increased.

In some embodiments, an assay may be performed at a range of PCR themocycling times and temperatures, with varying enzyme mixtures and concentrations, and different concentrations of Mg²⁺, Mn²⁺, Ca²⁺, Zn²⁺.

In some embodiments, reporter modifications are introduced, which include but are not limited to the use of Fluorescence Resonance Energy Transfer (FRET) or quenching in combination with one or more abasic-containing primers as shown in FIG. 8. Primer pairs usually include a fluorescent moiety and a quencher moiety. Examples can include but are not limited to FAM and Black Hole Quencher, FAM and Iowa Black Quencher, FAM and TAMRA, and FAM and ROX.

In some embodiments, other lesions, in addition to the abasic lesions, are extended by Sulfolobus DNA polymerase and cleaved by Tth Endonuclease IV. Non-limiting examples of additional lesion sites include urea sites, bulky bases, DNA adducts, base pair mismatches, flap and pseudo Y structures, and small insertions/deletions in DNA molecules.

The assay is not limited to the use of Tth Endonuclease IV. Any suitable thermostable endonuclease can be used. Non-limiting examples of thermostable endonucleases that can be used in an assay (e.g., can cleave an abasic site introduced via the primer) include Tma Endonuclease III (NEB, Ipswich Mass.) and Endonuclease III (Nth). The Tma Endonuclease III contains N-glyocosylase activity in addition to the endonuclease activity. The N-glyocosylase activity can be combined in an assay along with the endonuclease activity, in certain embodiments. The N-glyocosylase activity can release the base from pyrimidine lesion such as a uracil moiety leaving an abasic site, in some embodiments. The endonuclease activity can cleave the resulting abasic site.

In certain embodiments, non-thermostable or thermostable endonucleases can be used in a 2-step assay wherein the PCR amplification and endonuclease activity are performed separately.

The initial PCR is performed without an endonuclease. The endonuclease can be added post-PCR, and the reaction can be held at a temperature permissive for the endonuclease activity.

In some embodiments, a non-thermostable or thermostable lesion by-pass DNA polymerase can be used in a 2-step assay where the PCR amplification and endonuclease activity are performed separately. The initial PCR is performed with a non-lesion by-pass DNA polymerase, for example Taq DNA polymerase. The lesion by-pass DNA polymerase is added post-PCR and the reaction is held at a temperature permissive for the lesion-bypass activity.

In certain embodiments, a non-thermostable or thermostable lesion by-pass DNA polymerase and endonucleases can be used in a 2-step assay where the PCR amplification and endonuclease activity are performed separately. The initial PCR is performed without endonuclease and with a non-lesion by-pass DNA polymerase, for example Taq DNA polymerase. The lesion by-pass DNA polymerase and endonuclease are added post-PCR and the reaction is held at a temperature permissive for the lesion-bypass activity.

Any suitable non-thermostable endonuclease and or non-thermostable lesion bypass DNA polymerase can be used in the embodiments described above. Non-limiting examples of non-thermostable endonucleases include but are not limited to E. coli Endonuclease IV (NEB, Ipswich Mass.), E. coli Endonuclease III (NEB, Ipswich Mass.), E. coli Endonuclease VIII (NEB, Ipswich Mass.). E. coli DNA polymerase V is a Non-limiting example of a non-thermostable lesion bypass DNA polymerase.

### Example 12

### Examples of Certain Embodiments

Provided hereafter are non-limiting examples of certain embodiments. Certain embodiments are referenced non-sequentially.

A1. A method for amplifying a target nucleic acid, or portion thereof, in a nucleic acid composition, which comprises:
(a) contacting, under hybridization conditions, a nucleic acid composition with two oligonucleotide species, wherein each oligonucleotide species comprises:
   (i) a nucleotide subsequence complementary to the target nucleic acid,
   (ii) a non-terminal and non-functional portion of a first endonuclease cleavage site, wherein the portion of the first endonuclease cleavage site forms a functional first endonuclease cleavage site when the oligonucleotide species is hybridized to the target nucleic acid, and
   (iii) a blocking moiety at the 3′ end of the oligonucleotide species;
(b) cleaving the first functional cleavage site with a first endonuclease under cleavage conditions, thereby generating an extendable primer and a fragment comprising the blocking moiety; and
(c) extending the extendable primer under amplification conditions, whereby the target nucleic acid, or portion thereof, is amplified.

A2. The method of embodiment A1, wherein the fragment comprising the blocking moiety comprises a detectable feature.

A3. The method of embodiment A2, which further comprises detecting the detectable feature.

A4. The method of embodiment A2 or A3, wherein the fragment comprising the blocking moiety comprises a capture agent.

A5. The method of any one of embodiments A1-A4, wherein the blocking moiety of a first oligonucleotide species is different than the blocking moiety of a second oligonucleotide species.

A6. The method of any one of embodiments A1-A5, wherein the blocking moiety of each oligonucleotide species independently is selected from the group consisting of biotin, avidin, streptavidin and a detectable label.

A7. The method of any one of embodiments A1-A6, wherein (a), (b) and (c) are performed in the same reaction environment and/or are performed contemporaneously.

A8. The method of any one of embodiments A1-A7, wherein one of the oligonucleotide species comprises a 5′ region, wherein the 5′ region comprises:
(i) a nucleotide subsequence not complementary to the target nucleic acid,
(ii) a non-functional portion of a second endonuclease cleavage site, whereby the non-functional portion of the second endonuclease cleavage site is converted into a functional second endonuclease cleavage site under the amplification conditions, and
(iii) a detectable feature.

A9. The method of embodiment A8, which further comprises cleaving the functional second endonuclease cleavage site with a second endonuclease under cleavage conditions, thereby generating a fragment comprising the detectable feature.

A10. The method of embodiment A9, wherein the cleaving generates two or more fragments comprising distinguishable detectable features.

A11. The method of embodiment A9 or A10, which further comprises detecting one or more of the detectable features of one or more of the fragments.

A12. The method of embodiment A9 or A10, wherein one or more of the fragments comprise a capture agent.

A13. The method of any one of embodiments A8-A13, wherein the cleaving with the second endonuclease is performed in the same reaction environment as (a), (b) and (c), and/or is performed contemporaneously with (a), (b) and (c).

A50. A method for detecting a target nucleic acid in a nucleic acid composition, which comprises:
(a) contacting, under hybridization conditions, a nucleic acid composition with two oligonucleotide species, wherein each oligonucleotide species comprises:
   (i) a nucleotide subsequence complementary to the target nucleic acid,
   (ii) a non-terminal and non-functional portion of a first endonuclease cleavage site, wherein the portion of the first endonuclease cleavage site forms a functional first endonuclease cleavage site when the oligonucleotide species is hybridized to the target nucleic acid,
   (iii) a detectable feature, and
   (iv) a blocking moiety at the 3′ end of the oligonucleotide species;
(b) contacting, under cleavage conditions, the nucleic acid composition with a first endonuclease, wherein the first endonuclease cleaves the functional first endonuclease cleavage site when target nucleic acid is present, thereby generating and releasing a cleavage product having the detectable feature; and
(c) detecting the presence or absence of the cleavage product having the detectable feature, whereby the presence or absence of the target nucleic acid is detected based on detecting the presence or absence of the cleavage product with the detectable feature.

A51. The method of embodiment A50, wherein (a) and (b) are performed in the same reaction environment.

A52. The method of embodiment A50 or A51, wherein (a) and (b) are performed contemporaneously.

A53. The method of any one of embodiments A50-A52, wherein the cleaving in (b) generates two or more cleavage products comprising distinguishable detectable features.

A54. The method of embodiment A53, wherein one or more of the detectable features of one or more of the cleavage products are detected.

A55. The method of any one of embodiments A50-A54, wherein one or more of the cleavage products comprise a capture agent.

A60. A method for detecting a target nucleic acid in a nucleic acid composition, which comprises:
(a) contacting, under hybridization conditions, a nucleic acid composition with two oligonucleotide species, wherein each oligonucleotide species comprises:
   (i) a nucleotide subsequence complementary to the target nucleic acid,
   (ii) a non-terminal and non-functional portion of a first endonuclease cleavage site, wherein the portion of the first endonuclease cleavage site forms a functional first endonuclease cleavage site when the oligonucleotide species is hybridized to the target nucleic acid,
   (iii) a detectable feature, and
   (iv) a blocking moiety at the 3′ end of the oligonucleotide species, and wherein one of the oligonucleotide species comprises a non-functional portion of a second endonuclease cleavage site;
(b) cleaving the first functional cleavage site with a first endonuclease under cleavage conditions, thereby generating an extendable primer;
(c) extending the extendable primer under amplification conditions, whereby the non-functional portion of the second endonuclease cleavage site is converted into a functional second endonuclease cleavage site under the amplification conditions;
(d) cleaving the functional second endonuclease cleavage site with a second endonuclease under cleavage conditions, thereby generating a cleavage product having the detectable feature; and
(e) detecting the presence or absence of the cleavage product having the detectable feature, whereby the presence or absence of the target nucleic acid is detected based on detecting the presence or absence of the cleavage product with the detectable feature.

A61. The method of embodiment A61, wherein (a), (b), (c) and (d) are performed in the same reaction environment.

A62. The method of embodiment A60 or A61, wherein (a), (b), (c) and (d) are performed contemporaneously.

A63. The method of any one of embodiments A60-A62, wherein the cleaving in (b) generates two or more cleavage products comprising distinguishable detectable features.

A64. The method of embodiment A63, wherein one or more of the detectable features of one or more of the cleavage products are detected.

A65. The method of any one of embodiments A60-A64, wherein one or more of the cleavage products comprise a capture agent.

B1. A method for amplifying a target nucleic acid, or portion thereof, in a nucleic acid composition, which comprises:
(a) contacting, under hybridization conditions, a nucleic acid composition with an oligonucleotide and forward and reverse polynucleotide primers, wherein:
   (i) the oligonucleotide comprises a nucleotide subsequence complementary to the target nucleic acid,
   (ii) the oligonucleotide comprises a non-terminal and non-functional portion of a first endonuclease cleavage site, wherein the portion of the first endonuclease cleavage site forms a functional first endonuclease cleavage site when the oligonucleotide species is hybridized to the target nucleic acid,
   (iii) the oligonucleotide comprises a blocking moiety at the 3′ end of the oligonucleotide species,
   (iv) one of the polynucleotide primers hybridizes to the target nucleic acid 5′ of the oligonucleotide;
(b) cleaving the first functional cleavage site with a first endonuclease under cleavage conditions, thereby generating cleavage products; and
(c) extending the polynucleotide primers under amplification conditions, whereby the target nucleic acid, or portion thereof, is amplified.

B2. The method of embodiment B1, wherein the oligonucleotide blocks extension of the polynucleotide primer until the first functional cleavage site is cleaved by the first endonuclease.

B3. The method of embodiment B1 or B2, wherein (a), (b) and (c) are performed in the same reaction environment.

B4. The method of any one of embodiments B1-B3, wherein (a), (b) and (c) are performed contemporaneously.

B5. The method of any one of embodiments B1-B4, wherein one or more cleavage products include a detectable feature.

B6. The method of embodiment B5, which further comprises detecting the detectable feature in the one or more cleavage products.

B7. The method of any one of embodiments B1-B6, wherein one or more cleavage products include a capture agent.

B50. A method for determining the presence or absence of a target nucleic acid in a nucleic acid composition, which comprises:
(a) contacting, under hybridization conditions, a nucleic acid composition with an oligonucleotide comprising:
   (i) a nucleotide subsequence complementary to the target nucleic acid,
   (ii) a non-terminal and non-functional portion of an endonuclease cleavage site, wherein the portion of the endonuclease cleavage site forms a functional endonuclease cleavage site when the oligonucleotide is hybridized to the target nucleic acid,
   (iii) a blocking moiety at the 3′ end of the oligonucleotide, and
   (iv) a detectable feature;
(b) contacting the nucleic acid composition with an endonuclease capable of cleaving the cleavage site under cleavage conditions, thereby generating oligonucleotide fragments having the detectable feature when the target nucleic acid is present; and
(c) detecting the presence or absence of the oligonucleotide fragments having the detectable feature, whereby the presence or absence of the target nucleic acid is determined based upon detecting the presence or absence of the oligonucleotide fragments.

B51. The method of embodiment B50, which comprises contacting the nucleic acid composition in (a) with two or more oligonucleotide species.

B52. The method of embodiment B50 or B51, wherein (a) and (b) are performed in the same reaction environment.

B53. The method of any one of embodiments B50-B52, wherein (a) and (b) are performed contemporaneously.

B54. The method of any one of embodiments B50-B63, wherein the cleaving in (b) generates two or more oligonucleotide fragments comprising distinguishable detectable features.

B55. The method of embodiment B54, wherein one or more of the detectable features of one or more of the oligonucleotide fragments are detected.

B56. The method of any one of embodiments B50-B55, wherein one or more of the oligonucleotide fragments comprise a capture agent.

B60. A method for determining the presence or absence of a target nucleic acid in a nucleic acid composition, which comprises:
(a) contacting, under hybridization conditions, a nucleic acid composition with an oligonucleotide comprising:
   (i) a nucleotide subsequence complementary to the target nucleic acid,
   (ii) a non-terminal and non-functional portion of an endonuclease cleavage site, wherein the portion of the endonuclease cleavage site forms a functional endonuclease cleavage site when the oligonucleotide is hybridized to the target nucleic acid,
   (iii) a blocking moiety at the 3′ end of the oligonucleotide, and
   (iv) a detectable feature;
(b) contacting the nucleic acid composition with an endonuclease capable of cleaving the cleavage site under cleavage conditions, thereby generating oligonucleotide fragments having the detectable feature when the target nucleic acid is present;
(c) contacting the nucleic acid composition with forward and reverse primer polynucleotides under extension conditions; and
(d) detecting the presence or absence of the oligonucleotide fragments having the detectable feature, whereby the presence or absence of the target nucleic acid is determined based upon detecting the presence or absence of the oligonucleotide fragments.

B61. The method of embodiment B60, which comprises contacting the nucleic acid composition in (a) with two or more oligonucleotide species.

B62. The method of embodiment B60 or B61, wherein (a), (b) and (c) are performed in the same reaction environment.

B63. The method of any one of embodiments B60-B62, wherein (a), (b) and (c) are performed contemporaneously.

B64. The method of any one of embodiments B60-B63, wherein the cleaving in (b) generates two or more oligonucleotide fragments comprising distinguishable detectable features.

B65. The method of embodiment B64, wherein one or more of the detectable features of one or more of the oligonucleotide fragments are detected.

B66. The method of any one of embodiments B60-B65, wherein one or more of the oligonucleotide fragments comprise a capture agent.

C1. A method for amplifying a target nucleic acid, or portion thereof, in a nucleic acid composition, which comprises:
(a) contacting, under hybridization conditions, a nucleic acid composition with an oligonucleotide and a primer polynucleotide, wherein the oligonucleotide comprises:
   (i) a nucleotide subsequence complementary to the target nucleic acid, and
   (ii) a non-terminal and non-functional portion of a first endonuclease cleavage site; and
(b) extending the oligonucleotide under amplification conditions, thereby generating an extended oligonucleotide, wherein the primer polynucleotide hybridizes to the extended oligonucleotide and is extended under the amplification conditions, thereby yielding a double-stranded amplification product that comprises a functional first endonuclease cleavage site, whereby the target nucleic acid, or portion thereof, is amplified.

C2. The method of embodiment C1, which further comprises (c) cleaving the first functional cleavage site with a first endonuclease under cleavage conditions, thereby generating a double-stranded cleavage product.

C3. The method of embodiment C1 or C2, wherein the double-stranded cleavage product comprises a detectable feature.

C4. The method of embodiment C3, which further comprises detecting the detectable feature.

C5. The method of embodiment C3 or C4, wherein the double-stranded cleavage product comprises a capture agent.

C6. The method of any one of embodiments C1-C5, wherein (a) and (b) are performed in the same reaction environment.

C7. The method of any one of embodiments C1-C6, wherein (a) and (b) are performed contemporaneously.

C8. The method of embodiment Cl, which further comprises (c) cleaving the first functional cleavage site with a first endonuclease under cleavage conditions, thereby generating a single-stranded cleavage product.

C9. The method of embodiment C1 or C8, wherein the single-stranded cleavage product comprises a detectable feature.

C10. The method of embodiment C9, which further comprises detecting the detectable feature.

C11. The method of embodiment C9 or C10, wherein the single-stranded cleavage product comprises a capture agent.

C12. The method of any one of embodiments C1 to C11, wherein the first endonuclease cleavage site comprises an abasic site.

C13. The method of embodiment C12, wherein the amplification conditions comprise a trans-lesion synthesizing polymerase.

C14. The method of embodiment C13, wherein the polymerase is a trans-lesion Y-family polymerase.

C15. The method of embodiment C14, wherein the polymerase is a Sulfolobus DNA Polymerase IV.

C50. A method for detecting the presence or absence of a target nucleic acid in a nucleic acid composition, which comprises:
(a) contacting, under hybridization conditions, a nucleic acid composition with an oligonucleotide and a primer polynucleotide, wherein the oligonucleotide comprises:
   (i) a nucleotide subsequence complementary to the target nucleic acid,
   (ii) a non-terminal and non-functional portion of a first endonuclease cleavage site, and
   (iii) a detectable feature; and
(b) exposing the nucleic acid composition to amplification conditions, wherein (i) the oligonucleotide is extended when the target nucleic acid is present, and (ii) the primer polynucleotide hybridizes to the extended oligonucleotide and is extended under the amplification conditions, thereby yielding a double-stranded amplification product that comprises a functional first endonuclease cleavage site;
(c) contacting the nucleic acid composition with a first endonuclease that cleaves the functional first endonuclease cleavage site, thereby generating a cleavage product comprising the detectable feature; and
(d) detecting the presence or absence of the cleavage product comprising the detectable feature, whereby the presence or absence of the target nucleic acid is detected based on the presence or absence of the cleavage product comprising the detectable feature.

C51. The method of embodiment C50, wherein (a), (b) and (c) are performed in the same reaction environment.

C52. The method of embodiment C50 or C51, wherein (a), (b) and (c) are performed contemporaneously.

C53. The method of any one of embodiments C50-C52, wherein the cleaving in (c) generates two or more cleavage products comprising distinguishable detectable features.

C54. The method of embodiment C53, wherein one or more of the detectable features of one or more of the cleavage products are detected.

C55. The method of any one of embodiments C50-C54, wherein one or more of the cleavage products comprise a capture agent.

C56. The method of any one of embodiments C50 to C55, wherein the first endonuclease cleavage site comprises an abasic site.

C57. The method of embodiment C56, wherein the amplification conditions comprise a trans-lesion synthesizing polymerase.

C58. The method of embodiment C57, wherein the polymerase is a trans-lesion Y-family polymerase.

C59. The method of embodiment C58, wherein the polymerase is a Sulfolobus DNA Polymerase IV.

D1. A method for amplifying a target nucleic acid, or portion thereof, in a nucleic acid composition, which comprises:
(a) providing an oligonucleotide and a polynucleotide, or providing an oligonucleotide that includes a 3′ portion, under hybridization conditions, wherein:
   (i) the oligonucleotide comprises a nucleotide subsequence complementary to the target nucleic acid,
   (ii) the polynucleotide comprises a polynucleotide subsequence complementary to (“complementary polynucleotide sequence”) and hybridized to a complementary subsequence of the oligonucleotide,
   (iii) the 3′ portion of the oligonucleotide comprises a polynucleotide subsequence complementary to (“complementary polynucleotide sequence”) and hybridized to a 5′ complementary subsequence of the oligonucleotide, and
   (iv) the complementary subsequence of the oligonucleotide and the complementary polynucleotide sequence comprise a functional first endonuclease cleavage site;
(b) cleaving the first functional cleavage site with a first endonuclease under cleavage conditions, thereby generating an extendable primer oligonucleotide;
(c) contacting the nucleic acid composition with the extendable primer oligonucleotide;
(d) extending the extendable primer oligonucleotide under amplification conditions in the presence of a primer nucleic acid, wherein (i) an extended primer oligonucleotide is generated, and (ii) the primer nucleic acid hybridizes to the extended primer oligonucleotide and is extended,
whereby the target nucleic acid, or portion thereof, is amplified.

D2. The method of embodiment D1, wherein:
the oligonucleotide comprises a non-functional portion of a second endonuclease cleavage site, and
a double-stranded amplification product comprising a functional second endonuclease cleavage site is generated under the amplification conditions.

D3. The method of embodiment D2, which further comprises (e) cleaving the functional second endonuclease cleavage site with a second endonuclease, thereby generating a cleavage product.

D4. The method of embodiment D3, wherein the cleavage product is double-stranded (e.g., the endonuclease cleaves both strands of the double-stranded amplification product).

D5. The method of embodiment D3, wherein the cleavage product is single-stranded (e.g., the endonuclease cleaves one strand of the double-stranded amplification product).

D6. The method of any one of embodiments D3-D5, wherein the cleaving generates two or more cleavage products comprising distinguishable detectable features.

D7. The method of any one of embodiments D3-D6, wherein one or more of the detectable features of one or more of the cleavage products are detected.

D8. The method of any one of embodiments D3-D7, wherein one or more of the cleavage products comprise a capture agent.

D9. The method of any one of embodiments D1-D8, wherein the oligonucleotide and the polynucleotide comprise the same or a different blocking moiety.

D10. The method of any one of embodiments D1-D9, wherein (a), (b), (c) and (d), or (a), (b), (c), (d) and (e), are performed in the same reaction environment.

D11. The method of any one of embodiments D1-D10, wherein (a), (b), (c) and (d), or (a), (b), (c), (d) and (e), are performed contemporaneously.

D12. The method of any one of embodiments D1-D11, wherein the oligonucleotide that includes a 3′ portion forms a stem-loop structure.

D50. A method for detecting a target nucleic acid in a nucleic acid composition, which comprises:
(a) providing an oligonucleotide and a polynucleotide, or providing an oligonucleotide that includes a 3′ portion, under hybridization conditions, wherein:
   (i) the oligonucleotide comprises a nucleotide subsequence complementary to the target nucleic acid,
   (ii) the polynucleotide comprises a polynucleotide subsequence complementary to (“complementary polynucleotide sequence”) and hybridized to a complementary subsequence of the oligonucleotide,
   (iii) the 3′ portion of the oligonucleotide comprises a polynucleotide subsequence complementary to (“complementary polynucleotide sequence”) and hybridized to a 5′ complementary subsequence of the oligonucleotide,
   (iv) the complementary subsequence of the oligonucleotide and the complementary polynucleotide sequence comprise a functional first endonuclease cleavage site,
   (v) the oligonucleotide comprises a non-functional portion of a second endonuclease cleavage site, and
   (vi) the oligonucleotide comprises a detectable feature;
(b) providing a first endonuclease under cleavage conditions, wherein the first endonuclease cleaves the first endonuclease cleavage site, thereby generating an extendable primer oligonucleotide;
(c) contacting the nucleic acid composition with the extendable primer oligonucleotide;
(d) exposing the nucleic acid composition to amplification conditions and a primer nucleic acid, wherein: (i) the extendable primer oligonucleotide is extended when the target nucleic acid is present, thereby generating an extended primer oligonucleotide, and (ii) the primer nucleic acid hybridizes to the extended primer oligonucleotide and is extended, thereby generating a double-stranded amplification product comprising a functional second endonuclease cleavage site;
(e) contacting the nucleic acid composition with a second endonuclease under cleavage conditions, wherein the second endonuclease cleaves double-stranded amplification product comprising the functional second endonuclease cleavage site, thereby generating a cleavage product comprising the detectable feature; and
(f) detecting the presence or absence of the cleavage product comprising the detectable feature, whereby the presence or absence of the target nucleic acid is detected based on detecting the presence or absence of the cleavage product comprising the detectable feature.

D51. The method of embodiment D50, wherein (a), (b), (c), (d) and (e) are performed in the same reaction environment.

D52. The method of embodiment D50 or D51, wherein (a), (b), (c), (d) and (e) are performed contemporaneously.

D53. The method of any one of embodiments D50-D52, wherein the cleavage product is double-stranded (e.g., the endonuclease cleaves both strands of the double-stranded amplification product).

D54. The method of any one of embodiments D50-D53, wherein the cleavage product is single-stranded (e.g., the endonuclease cleaves one strand of the double-stranded amplification product).

D55. The method of any one of embodiments D50-D54, wherein the cleaving generates two or more cleavage products comprising distinguishable detectable features.

D56. The method of any one of embodiments D50-D55, wherein one or more of the detectable features of one or more of the cleavage products are detected.

D57. The method of any one of embodiments D50-D56, wherein one or more of the cleavage products comprise a capture agent.

E1. A method for determining the presence or absence of a target nucleic acid in a nucleic acid composition, which comprises:
(a) contacting the nucleic acid composition with an oligonucleotide, under hybridization conditions, wherein the oligonucleotide comprises:
   (i) the oligonucleotide comprises a terminal 5′ region, an internal 5′ region, an internal 3′ region and a terminal 3′ region,
   (ii) the oligonucleotide comprises a blocking moiety at the 3′ terminus, and
   (iii) the terminal 5′ region and the terminal 3′ region are substantially complementary to, and can hybridize to, the target nucleic acid,
   (iv) the internal 5′ region and the internal 3′ region are not complementary to the target nucleic acid,
   (v) the internal 5′ region is substantially complementary to the internal 3′ region and hybridize to one another to form an internal stem-loop structure when the terminal 5′ region and the terminal 3′ region are hybridized to the target nucleic acid,
   (vi) the internal 5′ region and the internal 3′ region do not hybridize to one another when the terminal 5′ region and the terminal 3′ region are not hybridized to the target nucleic acid, and
   (vii) the stem-loop structure comprises an endonuclease cleavage site;
(b) contacting the nucleic acid composition with an endonuclease capable of cleaving the cleavage site, whereby a stem-loop structure cleavage product is generated if the target nucleic acid is present in the nucleic acid composition; and
(c) detecting the presence or absence of the cleavage product, whereby the presence or absence of the target nucleic acid is determined based upon detecting the presence or absence of the cleavage product.

E2. The method of embodiment E1, wherein the cleavage product comprises a detectable feature.

E3. The method of embodiment E1 or E2, wherein the cleavage product comprises a capture agent.

E4. The method of any one of embodiments E1-E3, wherein (a) and (b) are performed in the same reaction environment.

E5. The method of any one of embodiments E1-E4, wherein (a) and (b) are performed contemporaneously.

F1. A method for determining the presence or absence of a target nucleic acid in a nucleic acid composition, which comprises:
(a) contacting the nucleic acid composition with a first oligonucleotide and a second oligonucleotide under hybridization conditions, wherein:
   (i) the first oligonucleotide and the second oligonucleotide each comprise a 5′ region, a 3′ region and a blocking moiety at the 3′ terminus,
   (ii) the 5′ region of the first oligonucleotide and the 3′ region of the second oligonucleotide are substantially complementary to, and can hybridize to, the target nucleic acid,
   (iii) the 3′ region of the first oligonucleotide and the 5′ region of the second oligonucleotide are not complementary to the target nucleic acid,
   (iv) the 3′ region of the first oligonucleotide is substantially complementary to the 5′ region of the second oligonucleotide are can hybridize to one another to form a stem structure when the 5′ region of the first oligonucleotide and the 3′ region of the second oligonucleotide are hybridized to the target nucleic acid,
   (v) the 3′ region of the first oligonucleotide and the 5′ region of the second oligonucleotide do not hybridize to one another when the 5′ region of the first oligonucleotide and the 3′ region of the second oligonucleotide are not hybridized to the target nucleic acid, and
   (vi) the stem structure comprises an endonuclease cleavage site;
(b) contacting the nucleic acid composition with an endonuclease capable of cleaving the cleavage site, whereby a stem structure cleavage product is generated if the target nucleic acid is present in the nucleic acid composition; and
(c) detecting the presence or absence of the cleavage product, whereby the presence or absence of the target nucleic acid is determined based upon detecting the presence or absence of the cleavage product.

F2. The method of embodiment F1, wherein the cleavage product comprises a detectable feature.

F3. The method of embodiment F1 or F2, wherein the cleavage product comprises a capture agent.

F4. The method of any one of embodiments F1-F3, wherein (a) and (b) are performed in the same reaction environment.

F5. The method of any one of embodiments F1-F4, wherein (a) and (b) are performed contemporaneously.

G1. The method of any one of the preceding applicable embodiments, wherein the capture agent is selected from the group consisting of biotin, avidin and streptavidin.

G2. The method of any one of the preceding applicable embodiments, wherein the endonuclease is thermostable.

G3. The method of embodiment G2, wherein the endonuclease loses less than about 50% of its maximum activity under the amplification conditions.

G4. The method of any one of the preceding applicable embodiments, wherein the endonuclease cleavage site includes an abasic site.

G5. The method of embodiment G4, wherein the endonuclease is an AP endonuclease.

G6. The method of any one of the preceding applicable embodiments, wherein the endonuclease is a restriction endonuclease.

G7. The method of embodiment G6, wherein the restriction endonuclease has double-stranded cleavage activity.

G8. The method of embodiment G6, wherein the restriction endonuclease has single-stranded cleavage activity (e.g., nicking enzyme).

G9. The method of any one of the preceding applicable embodiments, wherein the endonuclease cleaves DNA.

G10. The method of any one of the preceding applicable embodiments, wherein the endonuclease does not cleave RNA.

G11. The method of any one of the preceding applicable embodiments, wherein the endonuclease is not an RNase.

G12. The method of any one of the preceding applicable embodiments, wherein the oligonucleotide comprises one or more abasic sites.

G13. The method of any one of the preceding applicable embodiments, wherein the oligonucleotide comprises one or more non-cleavable bases.

G14. The method of embodiment G13, wherein the one or more non-cleavable bases are in a cleavage site, the restriction endonuclease has double-stranded cleavage activity, and the restriction endonuclease cleaves only one strand of the cleavage site.

G15. The method of any one of the preceding applicable embodiments, wherein the detectable feature is selected from the group consisting of mass, length, nucleotide sequence, optical property, electrical property, magnetic property, chemical property and time or speed through an opening in a matrix.

G16. The method of any one of the preceding applicable embodiments, wherein the detectable feature is mass.

G17. The method of embodiment G16, wherein the mass is detected by mass spectrometry.

G18. The method of embodiment G17, wherein the mass spectrometry is selected from the group consisting of Matrix-Assisted Laser Desorption/Ionization Time-of-Flight (MALDI-TOF) Mass Spectrometry (MS), Laser Desorption Mass Spectrometry (LDMS), Electrospray (ES) MS, Ion Cyclotron Resonance (ICR) MS, and Fourier Transform MS.

G19. The method of embodiment G17, wherein the mass spectrometry comprises ionizing and volatizing nucleic acid.

G20. The method of any one of the preceding applicable embodiments, wherein the detectable feature is a signal detected from a detectable label.

G21. The method of embodiment G20, wherein the signal is selected from the group consisting of fluorescence, luminescence, ultraviolet light, infrared light, visible wavelength light, light scattering, polarized light, radiation and isotope radiation.

G20. The method of any one of the preceding applicable embodiments, wherein the amplification conditions comprise a polymerase having strand displacement activity.

G21. The method of any one of the preceding applicable embodiments, wherein the blocking moiety is a 3′ terminal moiety selected from the group consisting of phosphate, amino, thiol, acetyl, biotin, cholesteryl, tetraethyleneglycol (TEG), biotin-TEG, cholesteryl-TEG, one or more inverted nucleotides, inverted deoxythymidine, digoxigenin, and 1,3-propanediol (C3 spacer).

G22. The method of any one of the preceding applicable embodiments, wherein the loop in the stem-loop structure comprises nucleotides.

G23. The method of any one of the preceding applicable embodiments, wherein the loop in the stem-loop structure comprises a non-nucleotide linker.

G24. The method of any one of the preceding applicable embodiments, wherein the stem in the stem-loop structure is partially single-stranded.

G25. The method of any one of the preceding applicable embodiments, wherein the stem in the stem-loop structure is double-stranded.

G26. The method of any one of the preceding applicable embodiments, wherein the stem-loop structure or stem structure comprises one or both members of a signal molecule pair, wherein the signal molecule pair members are separated by the endonuclease cleavage site.

G27. The method of embodiment G26, wherein the signal molecule pair members are fluorophore and quencher molecules.

G27. The method of embodiment G26, wherein the signal molecule pair members are fluorophore molecules suitable for fluorescence resonance energy transfer (FRET).

G28. The method of any one of the preceding applicable embodiments, wherein the first endonuclease is different than the second endonuclease.

G29. The method of any one of the preceding applicable embodiments, wherein amplification and/or extension conditions include a nucleic acid polymerase.

G30. The method of embodiment G29, wherein the nucleic acid polymerase is a DNA polymerase.

G31. The method of embodiment G29, wherein the nucleic acid polymerase is a RNA polymerase.

G32. The method of embodiment G29, wherein the polymerase is a trans-lesion synthesizing polymerase.

G33. The method of embodiment G32, wherein the polymerase is a trans-lesion Y-family polymerase.

G34. The method of embodiment G32, wherein the polymerase is a Sulfolobus DNA Polymerase IV.

G35. The method of embodiment G32, wherein the polymerase is capable of synthesizing DNA across one or more DNA template lesions.

G36. The method of embodiment G33, wherein the one or more lesions is one or more abasic sites.

G37. The method of embodiment G29, wherein the polymerase is selected from Taq DNA Polymerase; QBio™ Taq DNA Polymerase; SurePrime™ Polymerase; Arrow™ Taq DNA Polymerase; JumpStart Taq™; 9°N™m DNA polymerase; Deep Vent_{R}™ (exo-) DNA polymerase; Tth DNA polymerase; antibody-mediated polymerases; polymerases for thermostable amplification; native or modified RNA polymerases, and functional fragments thereof, native or modified DNA polymerases and functional fragments thereof, and combinations thereof.

G38. The method of any one of the preceding applicable embodiments, wherein the first endonuclease cleavage site comprises an abasic site.

G39. The method of embodiment G38, wherein the amplification conditions comprise a trans-lesion synthesizing polymerase.

G40. The method of embodiment C39, wherein the polymerase is a trans-lesion Y-family polymerase.

G41. The method of embodiment C40, wherein the polymerase is a Sulfolobus DNA Polymerase IV.

H1. A composition of matter comprising a blocked oligonucleotide that comprises:
(i) a non-terminal abasic site,
(ii) a blocking moiety at the 3′ terminus, and
(iii) a detectable feature.

I1. A composition of matter comprising two oligonucleotide species, wherein each oligonucleotide species comprises:
(i) a nucleotide subsequence complementary to a target nucleic acid,
(ii) a non-terminal and non-functional portion of a first endonuclease cleavage site, wherein the portion of the first endonuclease cleavage site forms a functional first endonuclease cleavage site when the oligonucleotide species is hybridized to the target nucleic acid, and
(iii) a blocking moiety at the 3′ end of the oligonucleotide species.

I2. The composition of embodiment I1, wherein one of the oligonucleotide species comprises a 5′ region that includes:
(i) a nucleotide subsequence not complementary to the target nucleic acid,
(ii) a non-functional portion of a second endonuclease cleavage site, whereby the non-functional portion of the second endonuclease cleavage site is converted into a functional second endonuclease cleavage site under amplification conditions, and
(iii) a detectable feature.

J1. A composition of matter that comprises an oligonucleotide and a polynucleotide hybridized to one another, wherein:
(i) the oligonucleotide comprises a nucleotide subsequence complementary to a target nucleic acid,
(ii) the polynucleotide comprises a polynucleotide subsequence complementary to (”complementary polynucleotide sequence“) and hybridized to a complementary subsequence of the oligonucleotide, and
(iii) the complementary subsequence of the oligonucleotide and the complementary polynucleotide sequence comprise a functional first endonuclease cleavage site.

J2. The composition of embodiment J1, wherein the oligonucleotide and the polynucleotide each comprise a blocking moiety at the 3′ terminus.

K1. A composition of matter that comprises an oligonucleotide and a polynucleotide hybridized to one another, wherein:
(i) the oligonucleotide comprises a nucleotide subsequence complementary to a target nucleic acid,
(ii) the polynucleotide comprises a polynucleotide subsequence complementary to (”complementary polynucleotide sequence“) and hybridized to a complementary subsequence of the oligonucleotide,
(iii) the complementary subsequence of the oligonucleotide and the complementary polynucleotide sequence comprise a functional first endonuclease cleavage site, and
(iv) the oligonucleotide comprises a non-functional portion of a second endonuclease cleavage site.

K2. The composition of embodiment K1, wherein the oligonucleotide and the polynucleotide each comprise a blocking moiety at the 3′ terminus.

L1. A composition of matter that comprises an oligonucleotide, wherein:
(i) the oligonucleotide comprises a nucleotide subsequence complementary to the target nucleic acid,
(ii) the oligonucleotide comprises a 3′ portion that comprises a polynucleotide subsequence complementary to (”complementary polynucleotide sequence“) and hybridized to a 5′ complementary subsequence of the oligonucleotide, thereby forming a stem-loop structure, and
(iii) the complementary subsequence of the oligonucleotide and the complementary polynucleotide sequence comprise a functional first endonuclease cleavage site.

L2. The composition of embodiment L1, wherein the oligonucleotide comprises a blocking moiety at the 3′ terminus.

M1. A composition of matter that comprises an oligonucleotide, wherein:
(i) the oligonucleotide comprises a nucleotide subsequence complementary to the target nucleic acid,
(ii) the oligonucleotide comprises a 3′ portion that comprises a polynucleotide subsequence complementary to (“complementary polynucleotide sequence”) and hybridized to a 5′ complementary subsequence of the oligonucleotide, thereby forming a stem-loop structure,
(iii) the complementary subsequence of the oligonucleotide and the complementary polynucleotide sequence comprise a functional first endonuclease cleavage site, and
(iv) the oligonucleotide comprises a non-functional portion of a second endonuclease cleavage site.

M2. The composition of embodiment L1, wherein the oligonucleotide comprises a blocking moiety at the 3′ terminus.

N1. A composition of matter that comprises an oligonucleotide, wherein:
(i) the oligonucleotide comprises a terminal 5′ region, an internal 5′ region, an internal 3′ region and a terminal 3′ region,
(ii) the oligonucleotide comprises a blocking moiety at the 3′ terminus, and
(iii) the terminal 5′ region and the terminal 3′ region are substantially complementary to, and can hybridize to, a target nucleic acid,
(iv) the internal 5′ region and the internal 3′ region are not complementary to the target nucleic acid,
(v) the internal 5′ region is substantially complementary to the internal 3′ region and hybridize to one another to form an internal stem-loop structure when the terminal 5′ region and the terminal 3′ region are hybridized to the target nucleic acid,
(vi) the internal 5′ region and the internal 3′ region do not hybridize to one another when the terminal 5′ region and the terminal 3′ region are not hybridized to the target nucleic acid, and
(vii) the stem-loop structure comprises an endonuclease cleavage site.

O1. A composition of matter that comprises a first oligonucleotide and a second oligonucleotide, wherein:
(i) the first oligonucleotide and the second oligonucleotide each comprise a 5′ region, a 3′ region and a blocking moiety at the 3′ terminus,
(ii) the 5′ region of the first oligonucleotide and the 3′ region of the second oligonucleotide are substantially complementary to, and can hybridize to, the target nucleic acid,
(iii) the 3′ region of the first oligonucleotide and the 5′ region of the second oligonucleotide are not complementary to the target nucleic acid,
(iv) the 3′ region of the first oligonucleotide is substantially complementary to the 5′ region of the second oligonucleotide are can hybridize to one another to form a stem structure when the 5′ region of the first oligonucleotide and the 3′ region of the second oligonucleotide are hybridized to the target nucleic acid,
(v) the 3′ region of the first oligonucleotide and the 5′ region of the second oligonucleotide do not hybridize to one another when the 5′ region of the first oligonucleotide and the 3′ region of the second oligonucleotide are not hybridized to the target nucleic acid, and
(vi) the stem structure comprises an endonuclease cleavage site.
The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the claimed technology. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” is about 1, about 2 and about 3). For example, a weight of “about 100 grams” can include weights between 90 grams and 110 grams. Thus, it should be understood that although the present technology has been specifically disclosed by representative embodiments and optional features, modification and variation of the concepts herein disclosed may be resorted to by those skilled in the art, and such modifications and variations are considered within the scope of this technology.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Some embodiments of the technology are set forth in the claims that follow.

What is claimed is:

1. A method for amplifying a target nucleic acid, or portion thereof, in a nucleic acid composition, which comprises:
(a) contacting, under hybridization conditions, a nucleic acid composition with an oligonucleotide and a primer polynucleotide, wherein the oligonucleotide comprises:
(i) a nucleotide subsequence complementary to the target nucleic acid, and
(ii) a non-terminal and non-functional portion of a first endonuclease cleavage site; and
(b) extending the oligonucleotide under amplification conditions, thereby generating an extended oligonucleotide, wherein the primer polynucleotide hybridizes to the extended oligonucleotide and is extended under the amplification conditions, thereby yielding a double-stranded amplification product that comprises a functional first endonuclease cleavage site, whereby the target nucleic acid, or portion thereof, is amplified.

2. The method of claim 1, which comprises (c) cleaving the first functional cleavage site with a first endonuclease under cleavage conditions, thereby generating a double-stranded cleavage product.

3. The method of claim 1, wherein the double-stranded cleavage product comprises a detectable feature.

4. The method of claim 3, which comprises detecting the detectable feature.

5. The method of claim 4, wherein the detectable feature is the mass of the double-stranded cleavage product.

6. The method of claim 1, wherein the double-stranded cleavage product comprises a capture agent.

7. The method of claim 1, wherein (a) and (b) are performed in the same reaction environment.

8. The method of claim 1, wherein (a) and (b) are performed contemporaneously.

9. The method of claim 1, which comprises (c) cleaving the first functional cleavage site with a first endonuclease under cleavage conditions, thereby generating a single-stranded cleavage product.

10. The method of claim 1, wherein the single-stranded cleavage product comprises a detectable feature.

11. The method of claim 10, which comprises detecting the detectable feature.

12. The method of claim 11, wherein the detectable feature is the mass of the single-stranded cleavage product.

13. The method of claim 9, wherein the single-stranded cleavage product comprises a capture agent.

14. The method of claim 13, wherein the capture agent is biotin, avidin or streptavidin.

15. The method of claim 1, wherein the first endonuclease cleavage site comprises an abasic site.

16. The method of claim 15, wherein the amplification conditions comprise a trans-lesion synthesizing polymerase.

17. The method of claim 16, wherein the polymerase is a trans-lesion Y-family polymerase.

18. The method of claim 17, wherein the polymerase is a Sulfolobus DNA Polymerase IV.

19. The method of claim 1, wherein two or more target nucleic acids are amplified.

20. A method for detecting the presence or absence of a target nucleic acid in a nucleic acid composition, which comprises:
(a) contacting, under hybridization conditions, a nucleic acid composition with an oligonucleotide and a primer polynucleotide, wherein the oligonucleotide comprises:
(i) a nucleotide subsequence complementary to the target nucleic acid,
(ii) a non-terminal and non-functional portion of a first endonuclease cleavage site, and
(iii) a detectable feature; and
(b) exposing the nucleic acid composition to amplification conditions, wherein (i) the oligonucleotide is extended when the target nucleic acid is present, and (ii) the primer polynucleotide hybridizes to the extended oligonucleotide and is extended under the amplification conditions, thereby yielding a double-stranded amplification product that comprises a functional first endonuclease cleavage site;
(c) contacting the nucleic acid composition with a first endonuclease that cleaves the functional first endonuclease cleavage site, thereby generating a cleavage product comprising the detectable feature; and
(d) detecting the presence or absence of the cleavage product comprising the detectable feature, whereby the presence or absence of the target nucleic acid is detected based on the presence or absence of the cleavage product comprising the detectable feature.

21. The method of claim 20, wherein (a), (b) and (c) are performed in the same reaction environment.

22. The method of claim 20, wherein (a), (b) and (c) are performed contemporaneously.

23. The method of claim 20, wherein the cleaving in (c) generates two or more cleavage products comprising distinguishable detectable features.

24. The method of claim 20, wherein one or more of the detectable features of one or more of the cleavage products are detected.

25. The method of claim 24, wherein the one or more of the detectable features is the mass of the cleavage products.

26. The method of claim 20, wherein one or more of the cleavage products comprise a capture agent.

27. The method of claim 26, wherein the capture agent is biotin, avidin or streptavidin.

28. The method of claim 20, wherein the first endonuclease cleavage site comprises an abasic site.

29. The method of claim 28, wherein the amplification conditions comprise a trans-lesion synthesizing polymerase.

30. The method of claim 29 wherein the polymerase is a trans-lesion Y-family polymerase.

31. The method of claim 30, wherein the polymerase is a Sulfolobus DNA Polymerase IV.

32. The method of claim 20, wherein the presence or absence of two or more target nucleic acids is detected in a multiplex analysis.
